(19) United States
(12) Patent Application Publication
(10) Pub. No.: US 2025/0243261 A1
(43) Pub. Date: Jul. 31, 2025

(54) ANTIBODY COMPOSITIONS AND OPTIMIZATION METHODS

(71) Applicants: CZ Biohub SF, LLC, San Francisco, CA (US); The Board of Trustees of the Leland Stanford Junior University, Stanford, CA (US)

(72) Inventors: Brian Lance Hie, San Francisco, CA (US); Varun Shanker, Stanford, CA (US); Peter S. Kim, Stanford, CA (US)

(73) Assignees: CZ Biohub SF, LLC, San Francisco, CA (US); The Board of Trustees of the Leland Stanford Junior University, Stanford, CA (US)

(21) Appl. No.: 18/854,205

(22) PCT Filed: Apr. 7, 2023
(86) PCT No.: PCT/US2023/017977

§ 371 Date: Oct. 4, 2024

Related U.S. Application Data
(60) Provisional application No. 63/329,218, filed on Apr. 8, 2022.

(51) Int. Cl. C07K 16/10; G01N 33/569; G16B 40/00

(52) U.S. Cl. CPC C07K 16/1003; C07K 16/10; C07K 16/1018; G01N 33/56983; G16B 40/00; C07K 2317/55; C07K 2317/76; C07K 2317/92; C07K 2317/94; G01N 2333/11; G01N 2333/165; G01N 2333/183

(57) ABSTRACT

Provided herein are methods using machine learning to predict protein variants that are likely to occur in nature. Such variants can be used (selected) to improve properties of the proteins. Also provided herein are antibodies and antigen binding portions thereof generated using the provided methods that specifically bind several antigens from coronaviruses, ebolaviruses, and influenza A viruses, various compositions of such antibodies or antigen binding portions thereof, recombinant nucleic acids encoding the antibodies and antigen binding portions thereof, and associated methods of use.

### CROSS-REFERENCES TO RELATED APPLICATIONS

The present application claims priority from and is a PCT application of U.S. Provisional Application No. 63/329,218, entitled “Antibody Compositions And Optimization Methods” filed Apr. 8, 2022, the entire contents of which are herein incorporated by reference for all purposes.

### BACKGROUND

An apparent paradox in evolutionary biology is how a random process can reliably generate new functions in short timescales across settings as diverse as antibody affinity maturation, viral immune escape, or tumor evolution. Current approaches for directed evolution of proteins in the laboratory are limited, as high-throughput evolutionary screens that rely on random guessing or brute-force search often devote substantial effort to interrogating weakly active or nonfunctional proteins. There is a need for more efficient, effective methods of generating protein variants using directed evolution.

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the creation by the inventors of methods for predicting antibody variants that are likely to occur in nature. Such variants can be used (selected) to improve properties of the antibodies, as described herein. The present disclosure is also based, in part, on antibody variants designed using the provided methods that are able to specifically bind to viral antigens.

In one aspect, provided herein are isolated antibodies or antigen-binding portions thereof comprising: a heavy chain variable region comprising (i) a CDRH1 comprising at least 90% identity to SEQ ID NO: 15; (ii) a CDRH2 comprising at least 90% identity to SEQ ID NO: 16; and (iii) a CDRH3 comprising at least 90% identity to SEQ ID NO: 17; and a light chain variable region comprising (i) a CDRL1 comprising at least 90% identity to SEQ ID NO:35; (ii) a CDRL2 comprising at least 90% identity to SEQ ID NO:36; and (iii) a CDRL3 comprising at least 90% identity to SEQ ID NO:37, wherein the heavy chain variable region comprises an amino acid residue substitution at at least one of positions 124, D27, S44, T53, E65, N74, P75, or M117, wherein the positions are numbered with respect to SEQ ID NO: 1, and/or wherein the light chain variable region comprises an amino acid residue substitution at at least one of positions T25, L29, T33, G55, R92, and G95, wherein the positions are numbered with respect to SEQ ID NO:2. In some embodiments, the heavy chain variable region comprises amino acid residue substitutions at positions E65 and M117, wherein the positions are numbered with respect to SEQ ID NO:1. In some embodiments, the heavy chain variable region comprises amino acid residue substitutions I24V, D27F, S44G, T53I, E65P, E65R, N74S, P75R, and/or M117Y, wherein the positions are numbered with respect to SEQ ID NO:1; and/or the light chain variable region comprises amino acid residue substitutions T25A, L29V, T33L, G55A, R92D, and/or G95P, wherein the positions are numbered with respect to SEQ ID NO:2. In some embodiments, the heavy chain variable region comprises amino acid residue substitutions: i) E65P and M117Y; or ii) E65R and M117Y, wherein the positions are numbered with respect to SEQ ID NO: 1.

In another aspect, provided herein are isolated antibodies or antigen-binding portions thereof comprising: a heavy chain variable region comprising (i) a CDRH1 comprising at least 90% identity to SEQ ID NO: 18; (ii) a CDRH2 comprising at least 90% identity to SEQ ID NO:19; and (iii) a CDRH3 comprising at least 90% identity to SEQ ID NO: 20; and a light chain variable region comprising (i) a CDRL1 comprising at least 90% identity to SEQ ID NO:38; (ii) a CDRL2 comprising at least 90% identity to SEQ ID NO:39; and (iii) a CDRL3 comprising at least 90% identity to SEQ ID NO:37, wherein the heavy chain variable region comprises an amino acid residue substitution at at least one of positions S44, T53, K58, V65, N74, and P75, wherein the positions are numbered with respect to SEQ ID NO:3, and/or wherein the light chain variable region comprises an amino acid residue substitution at at least one of positions N34 and G95, wherein the positions are numbered with respect to SEQ ID NO:4. In some embodiments, i) the heavy chain variable region comprises amino acid residue substitutions at positions K58 and V65; ii) the heavy chain variable region comprises amino acid residue substitutions at positions K58 and P75; iii) the heavy chain variable region comprises amino acid residue substitutions at positions V65 and P75; iv) the heavy chain variable region comprises amino acid residue substitutions at positions K58, V65, and P75; v) the heavy chain variable region comprises an amino acid residue substitution at position K58 and the light chain variable region comprises an amino acid residue substitution at position G95; vi) the heavy chain variable region comprises an amino acid residue substitution at position V65 and the light chain variable region comprises an amino acid residue substitution at position G95; vii) the heavy chain variable region comprises an amino acid residue substitution at position P75 and the light chain variable region comprises an amino acid residue substitution at position G95; viii) the heavy chain variable region comprises amino acid residue substitutions at positions K58 and V65 and the light chain variable region comprises an amino acid residue substitution at position G95; ix) the heavy chain variable region comprises amino acid residue substitutions at positions K58 and P75 and the light chain variable region comprises an amino acid residue substitution at position G95; x) the heavy chain variable region comprises amino acid residue substitutions at positions V65 and P75 and the light chain variable region comprises an amino acid residue substitution at position G95; or xi) the heavy chain variable region comprises amino acid residue substitutions at positions K58, V65, and P75 and the light chain variable region comprises an amino acid residue substitution at position G95, wherein the heavy chain variable region positions are numbered with respect to SEQ ID NO:3 and the light chain variable region positions are numbered with respect to SEQ ID NO4.

In some embodiments, the heavy chain variable region comprises amino acid residue substitutions S44G, T53I, K58S, V65P, N74S, and/or P75R, wherein the positions are numbered with respect to SEQ ID NO:3; and/or the light chain variable region comprises amino acid residue substitutions N34A and/or G95P, wherein the positions are numbered with respect to SEQ ID NO:4. In some embodiments, i) the heavy chain variable region comprises amino acid residue substitutions K58S and V65P; ii) the heavy chain variable region comprises amino acid residue substitutions K58S and P75R; iii) the heavy chain variable region comprises amino acid residue substitutions V65P and P75R; iv) the heavy chain variable region comprises amino acid residue substitutions K58S, V65P, and P75R; v) the heavy chain variable region comprises amino acid residue substitution K58S and the light chain variable region comprises amino acid residue substitution G95P; vi) the heavy chain variable region comprises amino acid residue substitution V65P and the light chain variable region comprises amino acid residue substitution G95P; vii) the heavy chain variable region comprises amino acid residue substitution P75R and the light chain variable region comprises amino acid residue substitution G95P; viii) the heavy chain variable region comprises amino acid residue substitutions K58S and V65P and the light chain variable region comprises amino acid residue substitution G95P; ix) the heavy chain variable region comprises amino acid residue substitutions K58S and P75R and the light chain variable region comprises amino acid residue substitution G95P; x) the heavy chain variable region comprises amino acid residue substitutions V65P and P75R and the light chain variable region comprises amino acid residue substitution G95P; or xi) the heavy chain variable region comprises amino acid residue substitutions K58S, V65P, and P75R and the light chain variable region comprises amino acid residue substitution G95P, wherein the heavy chain variable region positions are numbered with respect to SEQ ID NO:3 and the light chain variable region positions are numbered with respect to SEQ ID NO4.

In another aspect, provided herein are isolated antibodies or antigen-binding portions thereof comprising: a heavy chain variable region comprising (i) a CDRH1 comprising at least 90% identity to SEQ ID NO:21; (ii) a CDRH2 comprising at least 90% identity to SEQ ID NO:22; and (iii) a CDRH3 comprising at least 90% identity to SEQ ID NO: 23; and a light chain variable region comprising (i) a CDRL1 comprising at least 90% identity to SEQ ID NO:40; (ii) a CDRL2 comprising at least 90% identity to SEQ ID NO:41; and (iii) a CDRL3 comprising at least 90% identity to SEQ ID NO:42, wherein the heavy chain variable region comprises an amino acid residue substitution at at least one of positions M31, 141, D42, A68, E72, S79, and 1113, wherein the positions are numbered with respect to SEQ ID NO:5, and/or wherein the light chain variable region comprises an amino acid residue substitution at at least one of positions 119, F29, V43, S49, H70, and N90, wherein the positions are numbered with respect to SEQ ID NO:6. In some embodiments, i) the heavy chain variable region comprises amino acid residue substitutions at positions D42, A68, and S79; ii) the heavy chain variable region comprises amino acid residue substitutions at positions 141, D42, A68, S79, and 1113; iii) the heavy chain variable region comprises amino acid residue substitutions at positions A68 and 1113 and the light chain variable region comprises an amino acid residue substitution at position V43; iv) the heavy chain variable region comprises amino acid residue substitutions at positions A68, E72, S79, and 1113 and the light chain variable region comprises an amino acid residue substitution at position V43; v) the heavy chain variable region comprises amino acid residue substitutions at positions D42, A68, and S79 and the light chain variable region comprises an amino acid residue substitution at position V43; or vi) the heavy chain variable region comprises amino acid residue substitutions at positions I41, D42, A68, S79, and 1113 and the light chain variable region comprises an amino acid residue substitution at position V43, wherein the heavy chain variable region positions are numbered with respect to SEQ ID NO:5 and the light chain variable region positions are numbered with respect to SEQ ID NO:6.

In some embodiments, the heavy chain variable region comprises amino acid residue substitutions M31S, I41P, D42G, A68T, E72D, S79Y, and/or I113T, wherein the positions are numbered with respect to SEQ ID NO:5; and/or the light chain variable region comprises amino acid residue substitutions 119V, F29I, V43A, S49Y, H70D, and/or N90Q, wherein the positions are numbered with respect to SEQ ID NO:6. In some embodiments, i) the heavy chain variable region comprises amino acid residue substitutions D42G, A68T, and S79Y; ii) the heavy chain variable region comprises amino acid residue substitutions I41P, D42G, A68T, S79Y, and I113T; iii) the heavy chain variable region comprises amino acid residue substitutions A68T and I113T and the light chain variable region comprises amino acid residue substitution V43A; iv) the heavy chain variable region comprises amino acid residue substitutions A68T, E72D, S79Y, and I113T and the light chain variable region comprises amino acid residue substitution V43A; v) the heavy chain variable region comprises amino acid residue substitutions D42G, A68T, and S79Y and the light chain variable region comprises amino acid residue substitution V43A; or vi) the heavy chain variable region comprises amino acid residue substitutions I41P, D42G, A68T, S79Y, and I113Y and the light chain variable region comprises amino acid residue substitution V43A, wherein the heavy chain variable region positions are numbered with respect to SEQ ID NO:5 and the light chain variable region positions are numbered with respect to SEQ ID NO:6.

In another aspect, provided herein are isolated antibodies or antigen-binding portions thereof comprising: a heavy chain variable region comprising (i) a CDRH1 comprising at least 90% identity to SEQ ID NO:24; (ii) a CDRH2 comprising at least 90% identity to SEQ ID NO:25; and (iii) a CDRH3 comprising at least 90% identity to SEQ ID NO: 23; and a light chain variable region comprising (i) a CDRL1 comprising at least 90% identity to SEQ ID NO:43; (ii) a CDRL2 comprising at least 90% identity to SEQ ID NO:44; and (iii) a CDRL3 comprising at least 90% identity to SEQ ID NO:45, wherein the heavy chain variable region comprises an amino acid residue substitution at at least one of positions T41, A54, P60, G61, E72, G88, and V96, wherein the positions are numbered with respect to SEQ ID NO:7, and/or wherein the light chain variable region comprises an amino acid residue substitution at at least one of positions V43 and K90, wherein the positions are numbered with respect to SEQ ID NO:8. In some embodiments, i) the heavy chain variable region comprises amino acid residue substitutions at positions P60 and G61; ii) the heavy chain variable region comprises amino acid residue substitutions at positions T41, P60, G61, E72, G88, and V96; iii) the heavy chain variable region comprises an amino acid residue substitution at position G88 and the light chain variable region comprises an amino acid residue substitution at position V43; iv) the heavy chain variable region comprises an amino acid residue substitution at position V96 and the light chain variable region comprises an amino acid residue substitution at position V43; v) the heavy chain variable region comprises amino acid residue substitutions at positions P60 and G61 and the light chain variable region comprises an amino acid residue substitution at position V43; vi) the heavy chain variable region comprises amino acid residue substitutions at positions P60, G61, G88, and V96 and the light chain variable region comprises an amino acid residue substitution at position V43; or vii) the heavy chain variable region comprises amino acid residue substitutions at positions T41, P60, G61, E72, G88, and V96 and the light chain variable region comprises an amino acid residue substitution at position V43, wherein the heavy chain variable region positions are numbered with respect to SEQ ID NO:7 and the light chain variable region positions are numbered with respect to SEQ ID NO: 8.

In some embodiments, the heavy chain variable region comprises amino acid residue substitutions T41P, A54G, P60A, G61D, E72D, G88E, and/or V96A, wherein the positions are numbered with respect to SEQ ID NO:7; and/or the light chain variable region comprises amino acid residue substitutions V43A and/or K90Q, wherein the positions are numbered with respect to SEQ ID NO:8. In some embodiments, i) the heavy chain variable region comprises amino acid residue substitutions P60A and G61D; ii) the heavy chain variable region comprises amino acid residue substitutions T41P, P60A, G61D, E72D, G88E, and V96A; iii) the heavy chain variable region comprises amino acid residue substitution G88E and the light chain variable region comprises amino acid residue substitution V43A; iv) the heavy chain variable region comprises amino acid residue substitution V96A and the light chain variable region comprises amino acid residue substitution V43A; v) the heavy chain variable region comprises amino acid residue substitutions P60A and G61D and the light chain variable region comprises amino acid residue substitution V43A; vi) the heavy chain variable region comprises amino acid residue substitutions P60A, G61D, G88E, and V96A and the light chain variable region comprises amino acid residue substitution V43A; or vii) the heavy chain variable region comprises amino acid residue substitutions T41P, P60A, G61D, E72D, G88E, and V96A and the light chain variable region comprises amino acid residue substitution V43A, wherein the heavy chain variable region positions are numbered with respect to SEQ ID NO:7 and the light chain variable region positions are numbered with respect to SEQ ID NO:8.

In another aspect, provided herein are isolated antibodies or antigen-binding portions thereof comprising: a heavy chain variable region comprising (i) a CDRH1 comprising at least 90% identity to SEQ ID NO:26; (ii) a CDRH2 comprising at least 90% identity to SEQ ID NO:27; and (iii) a CDRH3 comprising at least 90% identity to SEQ ID NO:28; and a light chain variable region comprising (i) a CDRL1 comprising at least 90% identity to SEQ ID NO:46; (ii) a CDRL2 comprising at least 90% identity to SEQ ID NO:47; and (iii) a CDRL3 comprising at least 90% identity to SEQ ID NO:48, wherein the heavy chain variable region comprises an amino acid residue substitution at at least one of positions P28, T77, G79, R84, R85, and R87, wherein the positions are numbered with respect to SEQ ID NO:9, and/or wherein the light chain variable region comprises an amino acid residue substitution at at least one of positions T28, T32, S95, and L96, wherein the positions are numbered with respect to SEQ ID NO: 10. In some embodiments, i) the heavy chain variable region comprises amino acid residue substitutions at positions T77, G79, and R84; ii) the heavy chain variable region comprises amino acid residue substitutions at positions T77, G79, R84, and R85; iii) the light chain variable region comprises amino acid residue substitutions at positions T28 and T32; iv) the heavy chain variable region comprises an amino acid residue substitution at position G79 and the light chain variable region comprises an amino acid residue substitution at position T28; v) the heavy chain variable region comprises an amino acid residue substitution at position R84 and the light chain variable region comprises an amino acid residue substitution at position T28; vi) the heavy chain variable region comprises an amino acid residue substitution at position R87 and the light chain variable region comprises an amino acid residue substitution at position T28; vii) the heavy chain variable region comprises amino acid residue substitutions at positions T77, G79, and R84 and the light chain variable region comprises an amino acid residue substitution at position T28; viii) the heavy chain variable region comprises amino acid residue substitutions at positions T77, G79, and R84 and the light chain variable region comprises amino acid residue substitutions at positions T28 and T32; or ix) the heavy chain variable region comprises amino acid residue substitutions at positions T77, G79, R84, and R85 and the light chain variable region comprises amino acid residue substitutions at positions T28 and T32, wherein the heavy chain variable region positions are numbered with respect to SEQ ID NO:9 and the light chain variable region positions are numbered with respect to SEQ ID NO: 10.

In some embodiments, the heavy chain variable region comprises amino acid residue substitutions P28T, T77N, G79A, R84S, R85S, and/or R87T, wherein the positions are numbered with respect to SEQ ID NO:9; and/or the light chain variable region comprises amino acid residue substitutions T28S, T32S, S95V, and/or L96P, wherein the positions are numbered with respect to SEQ ID NO:10. In some embodiments, i) the heavy chain variable region comprises amino acid residue substitutions T77N, G79A, and R84S; ii) the heavy chain variable region comprises amino acid residue substitutions T77N, G79A, R84S, and R85S; iii) the light chain variable region comprises amino acid residue substitutions T28S and T32S; iv) the heavy chain variable region comprises amino acid residue substitution G79A and the light chain variable region comprises amino acid residue substitution T28S; v) the heavy chain variable region comprises amino acid residue substitution R84S and the light chain variable region comprises amino acid residue substitution T28S; vi) the heavy chain variable region comprises amino acid residue substitution R87S and the light chain variable region comprises amino acid residue substitution T28S; vii) the heavy chain variable region comprises amino acid residue substitutions T77N, G79A, and R84S and the light chain variable region comprises amino acid residue substitution T28S; viii) the heavy chain variable region comprises amino acid residue substitutions T77N, G79A, and R84S and the light chain variable region comprises amino acid residue substitutions T28S and T32S; or ix) the heavy chain variable region comprises amino acid residue substitutions T77N, G79A, R84S, and R85S and the light chain variable region comprises amino acid residue substitutions T28S and T32S, wherein the heavy chain variable region positions are numbered with respect to SEQ ID NO:9 and the light chain variable region positions are numbered with respect to SEQ ID NO: 10.

In another aspect, provided herein are isolated antibodies or antigen-binding portions thereof comprising: a heavy chain variable region comprising (i) a CDRH1 comprising at least 90% identity to SEQ ID NO:29; (ii) a CDRH2 comprising at least 90% identity to SEQ ID NO:30; and (iii) a CDRH3 comprising at least 90% identity to SEQ ID NO:31; and a light chain variable region comprising (i) a CDRL1 comprising at least 90% identity to SEQ ID NO:49; (ii) a CDRL2 comprising at least 90% identity to SEQ ID NO:50; and (iii) a CDRL3 comprising at least 90% identity to SEQ ID NO:51, wherein the heavy chain variable region comprises an amino acid residue substitution at at least one of positions R16, S98, and V108, wherein the positions are numbered with respect to SEQ ID NO: 11, and/or wherein the light chain variable region comprises an amino acid residue substitution at at least one of positions S82, N91, L93, and 196, wherein the positions are numbered with respect to SEQ ID NO: 12. In some embodiments, i) the heavy chain variable region comprises amino acid residue substitutions at positions R16 and V108; ii) the light chain variable region comprises amino acid residue substitutions at positions S82, N91, and 196; iii) the heavy chain variable region comprises an amino acid residue substitution at position R16 and the light chain variable region comprises an amino acid residue substitution at position S82; iv) the heavy chain variable region comprises an amino acid residue substitution at position R16 and the light chain variable region comprises an amino acid residue substitution at position N91; v) the heavy chain variable region comprises an amino acid residue substitution at position R16 and the light chain variable region comprises an amino acid residue substitution at position I96; or vi) the heavy chain variable region comprises amino acid residue substitutions at positions R16 and V108 and the light chain variable region comprises amino acid residue substitutions at positions S82, N91, and 196, wherein the heavy chain variable region positions are numbered with respect to SEQ ID NO: 11 and the light chain variable region positions are numbered with respect to SEQ ID NO:12.

In some embodiments, the heavy chain variable region comprises amino acid residue substitutions R16G, S98R, and/or V108D, wherein the positions are numbered with respect to SEQ ID NO:11; and/or the light chain variable region comprises amino acid residue substitutions S82A, N91C, N91S, L93Y, and/or I96S, wherein the positions are numbered with respect to SEQ ID NO:12. In some embodiments, i) the heavy chain variable region comprises amino acid residue substitutions R16G and V108D; ii) the light chain variable region comprises amino acid residue substitutions S82A, N91C, and I96S; iii) the heavy chain variable region comprises amino acid residue substitution R16G and the light chain variable region comprises amino acid residue substitution S82A; iv) the heavy chain variable region comprises amino acid residue substitution R16G and the light chain variable region comprises amino acid residue substitution N91C; v) the heavy chain variable region comprises amino acid residue substitution R16G and the light chain variable region comprises amino acid residue substitution I96S; or vi) the heavy chain variable region comprises amino acid residue substitutions R16G and V108D and the light chain variable region comprises amino acid residue substitutions S82A, N91C, and I96S, wherein the heavy chain variable region positions are numbered with respect to SEQ ID NO: 11 and the light chain variable region positions are numbered with respect to SEQ ID NO: 12.

In another aspect, provided herein are isolated antibodies or antigen-binding portions thereof comprising: a heavy chain variable region comprising (i) a CDRH1 comprising at least 90% identity to SEQ ID NO:32; (ii) a CDRH2 comprising at least 90% identity to SEQ ID NO:33; and (iii) a CDRH3 comprising at least 90% identity to SEQ ID NO:34; and a light chain variable region comprising (i) a CDRL1 comprising at least 90% identity to SEQ ID NO:52; (ii) a CDRL2 comprising at least 90% identity to SEQ ID NO:53; and (iii) a CDRL3 comprising at least 90% identity to SEQ ID NO:54, wherein the heavy chain variable region comprises an amino acid residue substitution at at least one of positions V29, K32, L51, D57, A77, and G91, wherein the positions are numbered with respect to SEQ ID NO: 13, and/or wherein the light chain variable region comprises an amino acid residue substitution at at least one of positions N27, T33, L34, Y41, G53, S57, G82, and A96, wherein the positions are numbered with respect to SEQ ID NO: 14. In some embodiments, i) the heavy chain variable region comprises amino acid residue substitutions at positions L51, A77, and G91; ii) the light chain variable region comprises amino acid residue substitutions at positions T33 and G53; iii) the light chain variable region comprises amino acid residue substitutions at positions N27, T33, L34, and G53; iv) the light chain variable region comprises amino acid residue substitutions at positions N27, T33, L34, Y41, G53, S57, and G82; v) the heavy chain variable region comprises amino acid residue substitutions at positions L51, A77, and G91 and the light chain variable region comprises an amino acid residue substitution at position T33; or vi) the heavy chain variable region comprises amino acid residue substitutions at positions L51, A77, and G91 and the light chain variable region comprises amino acid residue substitutions at positions N27, T33, L34, and G53, wherein the heavy chain variable region positions are numbered with respect to SEQ ID NO: 13 and the light chain variable region positions are numbered with respect to SEQ ID NO:14.

In some embodiments, the heavy chain variable region comprises amino acid residue substitutions V29F, K32Y, L51Y, D57T, A77T, and/or G91A, wherein the positions are numbered with respect to SEQ ID NO: 13; and/or the light chain variable region comprises amino acid residue substitutions N27S, T33N, L34Y, Y41H, G53V, S57P, G82A, and/or A96S, wherein the positions are numbered with respect to SEQ ID NO: 14. In some embodiments, i) the heavy chain variable region comprises amino acid residue substitutions L51Y, A77T, and G91A; ii) the light chain variable region comprises amino acid residue substitutions T33N and G53V; iii) the light chain variable region comprises amino acid residue substitutions N27S, T33N, L34Y, and G53V; iv) the light chain variable region comprises amino acid residue substitutions N27S, T33N, L34Y, Y41H, G53V, S57P, and G82A; v) the heavy chain variable region comprises amino acid residue substitutions L51Y, A77T, and G91A and the light chain variable region comprises amino acid residue substitution T33N; or vi) the heavy chain variable region comprises amino acid residue substitutions L51Y, A77T, and G91A and the light chain variable region comprises amino acid residue substitutions N27S, T33N, L34Y, and G53V, wherein the heavy chain variable region positions are numbered with respect to SEQ ID NO: 13 and the light chain variable region positions are numbered with respect to SEQ ID NO: 14.

In another aspect, provided herein are isolated antibodies or antigen-binding portions thereof comprising: (a) a heavy chain variable region comprising (i) a CDRH1 comprising at least 90% identity to SEQ ID NO:62; (ii) a CDRH2 comprising at least 90% identity to SEQ ID NO:63; and (iii) a CDRH3 comprising at least 90% identity to SEQ ID NO:64; and (b) a light chain variable region comprising (i) a CDRL1 comprising at least 90% identity to SEQ ID NO:68; (ii) a CDRL2 comprising at least 90% identity to SEQ ID NO:69; and (iii) a CDRL3 comprising at least 90% identity to SEQ ID NO:70, wherein the heavy chain variable region comprises an amino acid residue substitution at at least one of positions D88, V90, S62, V81, F24, 131, H99, T79, and 1105, wherein the positions are numbered with respect to SEQ ID NO:58, and/or wherein the light chain variable region comprises an amino acid residue substitution at at least one of positions A98, Q39, T5, K47, F51, K44, M49, E85, and Q6, wherein the positions are numbered with respect to SEQ ID NO:59. In some embodiments, the heavy chain variable region comprises amino acid residue substitutions at one or more positions D88, V90, S62, V81, F24, I31, H99, T70, and 1105. In some embodiments, the heavy chain variable region comprises amino acid residue substitutions at positions D88, V90, S62, V81, F24, I31, H99, and T70. In some embodiments, the heavy chain variable region comprises amino acid residue substitutions at positions D88, V90, S62, V81, F24, 131, and H99. In some embodiments, the heavy chain variable region comprises amino acid residue substitutions at positions D88, V90, S62, V81, F24, I31, and T70. In some embodiments, the heavy chain variable region comprises amino acid residue substitutions at positions D88, V90, S62, V81, F24, I31, and T70. In some embodiments, the heavy chain variable region comprises amino acid residue substitutions at positions D88, V90, S62, V81, I31, H99, and T70. In some embodiments, the light chain variable region comprises amino acid residue substitutions at one or more positions A98I, Q39K, T5Q, K47E, F51Y, K44E, M49L, E85A, and Q6S. In some embodiments, the heavy chain variable region comprises an amino acid residue substitution at position V90, and the light chain variable region comprises an amino acid residue substitution at position E85. In some embodiments, the heavy chain variable region comprises an amino acid residue substitution at position S62, and the light chain variable region comprises an amino acid residue substitution at position E85. In some embodiments, the heavy chain variable region comprises an amino acid residue substitution at position T70, and the light chain variable region comprises an amino acid residue substitution at position E85. In some embodiments, the heavy chain variable region comprises an amino acid residue substitution at position V90, and the light chain variable region comprises an amino acid residue substitution at position Q39. In some embodiments, the heavy chain variable region comprises an amino acid residue substitution at position S62, and the light chain variable region comprises an amino acid residue substitution at position Q39. In some embodiments, the heavy chain variable region comprises an amino acid residue substitution at position T70, and the light chain variable region comprises an amino acid residue substitution at position Q39.

In some embodiments of the above isolated antibodies or antigen-binding portions thereof, the heavy chain variable region comprises at least one of amino acid residue substitutions D88Q, V90S, S62N, V81T, F24Y, 131T, H99Y, and T70S, wherein the positions are numbered with respect to SEQ ID NO:58; and/or the light chain variable region comprises amino acid residue substitutions A98I, Q39K, T5Q, K47E, F51Y, K44E, M49L, E85A, and Q6S, wherein the positions are numbered with respect to SEQ ID NO:59. In some embodiments, the heavy chain variable region comprises one or more amino acid residue substitutions D89Q, V90S, S62N, V81T, F24Y, 131T, H98Y, T70S, and I105L. In some embodiments, the heavy chain variable region comprises amino acid residue substitutions D88Q, V90S, S62N, V81T, F24Y, 131T, H99Y, and T70S. In some embodiments, the heavy chain variable region comprises amino acid residue substitutions D88Q, V90S, S62N, V81T, F24Y, 131T, and H99Y. In some embodiments, the heavy chain variable region comprises amino acid residue substitutions D88Q, V90S, S62N, V81T, F24Y, H99Y, and T70S. In some embodiments, the heavy chain variable region comprises amino acid residue substitutions D88Q, V90S, S62N, V81T, F24Y, 131T, and T70S. In some embodiments, the heavy chain variable region comprises amino acid residue substitutions D88Q, V90S, S62N, V81T, 131T, H99Y, and T70S. In some embodiments, the light chain variable region comprises at least one amino acid residue substitutions A98I, Q39K, T5Q, K47E, F51Y, K44E, M49L, E85A, and Q6S. In some embodiments, the heavy chain variable region comprises an amino acid residue substitution V90S, and the light chain variable region comprises an amino acid residue substitution E85A. In some embodiments, the heavy chain variable region comprises an amino acid residue substitution S62N, and the light chain variable region comprises an amino acid residue substitution E85A. In some embodiments, the heavy chain variable region comprises an amino acid residue substitution T70S, and the light chain variable region comprises an amino acid residue substitution E85A. In some embodiments, the heavy chain variable region comprises an amino acid residue substitution V90S, and the light chain variable region comprises an amino acid residue substitution Q39K. In some embodiments, the heavy chain variable region comprises an amino acid residue substitution S62N, and the light chain variable region comprises an amino acid residue substitution Q39K. In some embodiments, the heavy chain variable region comprises an amino acid residue substitution T70S, and the light chain variable region comprises an amino acid residue substitution Q39K, wherein the heavy chain variable region positions are numbered with respect to SEQ ID NO:58 and the light chain variable region positions are numbered with respect to SEQ ID NO:59.

In some aspects, provided herein are isolated antibodies or antigen-binding portions thereof comprising: (a) a heavy chain variable region comprising (i) a CDRH1 comprising at least 90% identity to SEQ ID NO:65; (ii) a CDRH2 comprising at least 90% identity to SEQ ID NO:66; and (iii) a CDRH3 comprising at least 90% identity to SEQ ID NO:67; and (b) a light chain variable region comprising (i) a CDRL1 comprising at least 90% identity to SEQ ID NO:71; (ii) a CDRL2 comprising at least 90% identity to SEQ ID NO:72; and (iii) a CDRL3 comprising at least 90% identity to SEQ ID NO:73, wherein the heavy chain variable region comprises an amino acid residue substitution at at least one of positions T53, A61, and E10, wherein the positions are numbered with respect to SEQ ID NO:60, and/or wherein the light chain variable region comprises an amino acid residue substitution at at least one of positions N95, S85, S54, and M4, wherein the positions are numbered with respect to SEQ ID NO:61. In some embodiments, the heavy chain variable region comprises amino acid residue substitutions at one or more positions T53, A61, and E10. In some embodiments, the light chain variable region comprises amino acid residue substitutions at one or more positions N95, S85, S54, and M4. In some embodiments, the heavy chain variable region comprises an amino acid residue substitution at position T53, and the light chain variable region comprises an amino acid residue substitution at position N95. In some embodiments, the heavy chain variable region comprises an amino acid residue substitution at position Q82, and the light chain variable region comprises an amino acid residue substitution at position N95. In some embodiments, the heavy chain variable region comprises an amino acid residue substitution at position A61, and the light chain variable region comprises an amino acid residue substitution at position N95. In some embodiments, the heavy chain variable region comprises an amino acid residue substitution at position T53, and the light chain variable region comprises an amino acid residue substitution at position M4. In some embodiments, the heavy chain variable region comprises an amino acid residue substitution at position Q82, and the light chain variable region comprises an amino acid residue substitution at position M4. In some embodiments, the heavy chain variable region comprises an amino acid residue substitution at position A61, and the light chain variable region comprises an amino acid residue substitution at position M4. In some embodiments, the heavy chain variable region comprises an amino acid residue substitutions at positions A61 and T53.

In some embodiments of the above isolated antibodies or antigen-binding portions thereof, the heavy chain variable region comprises at least one of amino acid residue substitutions T53L, A61S, and E10Q, wherein the positions are numbered with respect to SEQ ID NO:60; and/or the light chain variable region comprises amino acid residue substitutions N95V, S85A, S54T, and M4V, wherein the positions are numbered with respect to SEQ ID NO:61. In some embodiments, the heavy chain variable region comprises one or more amino acid residue substitutions T53L, A61S, and E10Q In some embodiments, the light chain variable region comprises one or more amino acid residue substitutions N95V, S85A, S54T, and M4V. In some embodiments, the heavy chain variable region comprises an amino acid residue substitution T53L, and the light chain variable region comprises an amino acid residue substitution N95V. In some embodiments, the heavy chain variable region comprises an amino acid residue substitution Q82T, and the light chain variable region comprises an amino acid residue substitution N95V. In some embodiments, the heavy chain variable region comprises an amino acid residue substitution A61S, and the light chain variable region comprises an amino acid residue substitution N95V. In some embodiments, the heavy chain variable region comprises an amino acid residue substitution T53L, and the light chain variable region comprises an amino acid residue substitution M4V. In some embodiments, the heavy chain variable region comprises an amino acid residue substitution Q82T, and the light chain variable region comprises an amino acid residue substitution M4V. In some embodiments, the heavy chain variable region comprises an amino acid residue substitution A61S, and the light chain variable region comprises an amino acid residue substitution M4V. In some embodiments, the heavy chain variable region comprises an amino acid residue substitutions at positions A61S and T53L.

Also provided herein are recombinant nucleic acid molecules encoding an antibody or antigen binding portion thereof described herein. In some embodiments, the recombinant nucleic acid molecule is a synthetic sequence designed for expression in a host cell.

Also provided herein are DNA constructs comprising a recombinant nucleic acid molecule described herein operably linked to a promoter that drives expression in a host cell. Also provided herein are vectors comprising a recombinant nucleic acid molecule or DNA construct described herein. Also provided herein are host cells comprising a recombinant nucleic acid molecule, DNA construct, or vector described herein. In some embodiments, the host cell is a bacterial cell. In some embodiments, the host cell is a eukaryotic cell. Also provided herein are compositions comprising an antibody or antigen binding portion thereof described herein and a pharmaceutically acceptable carrier.

Also provided herein are methods of detecting the presence of a virus in a biological sample comprising: contacting said sample with an isolated antibody or antigen binding portion thereof described herein, and detecting an amount of binding of the isolated antibody or antigen binding portion thereof as a determination of the presence of the virus in the sample. In some embodiments, the virus is an influenza A virus, an ebolavirus, or a coronavirus.

Also provided herein are methods of treating a subject with a viral infection, the method comprising administering to the subject a pharmaceutically effective amount of a composition described herein. In some embodiments, the viral infection is an influenza A infection, an ebolavirus infection, or a coronavirus infection. In some embodiments, the coronavirus infection is a SARS-CoV-2 infection.

In another aspect, provided herein is a method comprising, performing by a computer system: loading, into a memory of the computer system, N machine learning language models, wherein N is an integer equal to or greater than 1; receiving an input antibody sequence of a starting antibody, the input antibody sequence comprised of input amino acids; for each of the N machine learning language models: executing the machine learning language model, using the input antibody sequence, to obtain a likelihood of each of a set of amino acids being at each of a plurality of positions in the input antibody sequence; for each of the plurality of positions and for each of a plurality of mutations from the set of amino acids, comparing the likelihood of the mutation at the position to the likelihood of the input amino acid at the position; and based on the comparison, identifying a set of candidate mutations that have a likelihood that is equal to or greater than the input amino acid in at least a threshold number of the N machine learning language models. In some embodiments, the method further comprises: for each candidate mutation of the set of candidate mutations: creating a mutated antibody having the mutation; and experimentally testing the mutated antibody to determine whether the mutated antibody has a same or improved property relative to the starting antibody, thereby identifying a first set of validated mutated antibodies having validated mutations.

In some embodiments, the method further comprises: for each of the validated mutations: creating multiple-mutated antibodies having a plurality of the validated mutations; and experimentally testing the multiple-mutated antibody to determine whether the mutated antibody has a same or improved property relative to the starting antibody or to the mutated antibodies, thereby identifying a second set of validated multiple-mutated antibodies having validated multiple-mutations. In some embodiments, the method further comprises: performing additional testing of the first set of validated mutated antibodies or the second set of validated multiple-mutated antibodies for additional properties. In some embodiments, the property is a binding affinity to a target molecule. In some embodiments, the plurality of positions are all the positions of the input antibody sequence. In some embodiments, the input antibody sequence is a wildtype sequence. In some embodiments, the set of amino acids is all 20 amino acids that comprise proteins in a human. In some embodiments, N is an integer equal to or greater than 3.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-1C show a demonstration of guiding evolution with protein language models, according to aspects of this disclosure. The top panel (FIGS. 1A-1B) shows two possible models for relating the space of mutations with high evolutionary plausibility, or intrinsic fitness (for example, the set of valid antibodies), to the space with high fitness under specific selection pressures, or extrinsic fitness (for example, the set of antibodies with high binding affinity to a specific antigen). Both models assume that mutations with high extrinsic fitness make up a rare subset of the full mutational space. Under the first model (FIG. 1A), mutations with high extrinsic fitness are also rare within the subset of mutations with high intrinsic fitness. Under the second model (FIG. 1B), when restricted to the regime of high intrinsic fitness, mutations with high extrinsic fitness become much more common. The bottom panel (FIG. 1C) shows that protein language models, trained on millions of natural protein sequences, learn amino-acid patterns that are likely to occur in nature, leading to the hypothesis that language-model likelihood approximates intrinsic fitness. Assuming that this is a good approximation, and if the second model (FIG. 1B) better describes nature, then a language model with no information about specific selection pressures can still efficiently guide evolution.

FIGS. 2-4 show language-model-guided affinity maturation of seven human antibodies, according to aspects of this disclosure. The left panels (FIGS. 2A, 3A, and 4A) of each row shows strip plots visualizing the two rounds of directed evolution conducted for each antibody. Each point represents a variant of the antibody plotted according to the fold-change in K_{d} from wildtype; a gray, dashed line is drawn at a fold change of 1, and the wildtype point is colored gray. MED18852 variants were screened against HA H4 Hubei, MED18852 UCA variants against HA H1 Solomon, mAb114 and mAb114 UCA variants against GP, S309 variants against Wuhan-Hu-1 S-6P, and REGN10987 and C143 variants against Beta S-2P. The center panels (FIGS. 2B, 3B, and 4B) of each row shows phylogenetic trees illustrating the evolutionary trajectories from wildtype to the highest-affinity variant(s) of each antibody. Nodes are annotated with the K_{d}s for different antigens and the Tₘ of the Fab; all K_{d}s are for the monovalent Fab versions except those of C143, which are for the bivalent IgGs. ML variant: machine-learning-guided variant; H1 Solo.: H1 Solomon; W1: Wuhan-Hu-1; B: Beta, 0: Omicron. The right panels (FIGS. 2C, 3C, and 4C) of each row shows avidity and affinity measurements obtained via biolayer interferometry (BLI) of IgGs and Fabs at the indicated concentrations binding to the indicated antigen. Selected BLI traces of the highest-affinity variants for the respective antigens are plotted alongside those of the wildtype variants.

FIGS. 5A-5B show that affinity-matured variants improve pseudovirus neutralization, according to aspects of this disclosure. The top panel (FIG. 5A) show that variants of C143 obtained from the language-model-guided affinity maturation campaign described herein demonstrate improved neutralization in a pseudovirus assay. For Beta pseudovirus, the best improvement is the 30-fold improvement of VL G53V; for D614G pseudovirus, the best improvement is the 20-fold improvement of VL T33N-G53V. Also see FIGS. 6A, 6B, and 7. The bottom panel (FIG. 5B) shows that fold-change in K_{d} correlates well with fold-change in IC₅₀ (Spearman r=0.82) across all designs tested, consistent with higher binding affinity contributing to improved viral neutralization.

FIGS. 6A, 6B, and 7 show pseudovirus neutralization of affinity-matured variants, according to aspects of this disclosure. Neutralization curves for wildtype antibodies (gray) and variants obtained by the language-model-guided affinity maturation campaigns are shown. Also see Table 9, Table 12, and Table 13 for corresponding IC₅₀s.

FIGS. 8A-8B show the efficient manifold hypothesis, according to aspects of this disclosure. The top panel (FIG. 8A) shows that the same strategy and language models used to acquire affinity-enhancing mutations to antibodies can also acquire high-fitness variants across diverse natural proteins and definitions of fitness (validated with data from high-throughput scanning mutagenesis assays). A substantial portion (12% to 40%) of language-model-guided (LM guided) mutations have high extrinsic fitness, which in many cases is also significantly enriched compared to the background percentage of high-fitness mutations; also see Table 17. ADRB2: adrenoreceptor beta 2; 3-la.: 3-lactamase; Env: envelope glycoprotein; infA: translation initiation factor 1; MAPK1: mitogen-activated protein kinase 1; PafA: phosphate-irrepressible alkaline phosphatase. The bottom panel (FIG. 8B) shows a depiction of the idea that intrinsic fitness forms a manifold represented in this cartoon by the rainbow road, where ascending corresponds to improving extrinsic fitness and descending corresponds to lowering extrinsic fitness. Under the efficient manifold hypothesis, this manifold of intrinsic fitness is narrow, therefore moving in any direction (for example, via random or brute-force mutagenesis) would most likely decrease extrinsic fitness or fall off the manifold entirely (represented by the green ball). However, if movement is constrained to the narrow manifold of intrinsic fitness (for example, when guided by a language model), then the chance of improving extrinsic fitness increases substantially (represented by the red ball).

FIG. 9 is a flowchart illustrating a method of using machine learning language model(s) to identify candidate mutations.

### TERMS

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e. at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

“Virus” is used in both the plural and singular senses. “Virion” refers to a single virus. For example, the expression “coronavirus virion” refers to a coronavirus particle.

Influenza A viruses are negative-sense, single-stranded RNA viruses of the family Orthomyxoviridae and the genus Alphainfluenzavirus. Strains of all subtypes of influenza A virus have been isolated from wild birds, although disease is uncommon. Some isolates of influenza A virus cause severe disease both in domestic poultry and, rarely, in humans. Occasionally, viruses are transmitted from wild aquatic birds to domestic poultry, and this may cause an outbreak or give rise to human influenza pandemics.

“Hemagglutinin” or “HA” is a homotrimeric integral membrane glycoprotein found on the surface of influenza viruses and is integral to influenza A infectivity. Hemagglutinin is a Class I Fusion Protein, having multifunctional activity as both an attachment factor (to bind influenza virus to sialic acid on the surface of target cells) and membrane fusion protein (to fuse the viral envelope with the late endosomal membrane of target cells). HA in influenza A has at least 18 different subtypes. These subtypes are named H1 through H18. H16 was discovered in 2004 on influenza A viruses isolated from black-headed gulls from Sweden and Norway. H17 was discovered in 2012 in fruit bats. Most recently, H18 was discovered in a Peruvian bat in 2013. The first three hemagglutinins, H1, H2, and H3, are found in human influenza viruses. By phylogenic similarity, the HA proteins are divided into 2 groups, with H1, H2, H5, H6, H8, H9, H11, H12, H13, H16, H17, and H18 belonging to group 1 and the rest in group 2. The serotype of influenza A virus is determined by the Hemagglutinin (HA) and Neuraminidase (NA) proteins present on its surface. Neuraminidase (NA) has 11 known subtypes, hence influenza virus is named as HIN1, H5N2 etc., depending on the combinations of HA and NA.

Ebolaviruses are a genus of negative-sense, single-stranded RNA viruses of the family Filoviridae. The six known ebolavirus species are named for the region where each was originally identified: Bundibugyo ebolavirus, Reston ebolavirus, Sudan ebolavirus, Tai Forest ebolavirus (originally Côte d'Ivoire ebolavirus), Zaire ebolavirus, and Bombali ebolavirus. The last is the most recent species to be named and was isolated from Angolan free-tailed bats in Sierra Leone. Each species of the genus Ebolavirus has one member virus, and four of these cause Ebola virus disease (EVD) in humans, a type of hemorrhagic fever having a very high case fatality rate. The Reston virus has caused EVD in other primates. Zaire ebolavirus has the highest mortality rate of the ebolaviruses and is responsible for the largest number of outbreaks of the six known species of the genus, including the 1976 Zaire outbreak and the outbreak with the most deaths (2014).

The ebolavirus glycoprotein (GP) is the only virally expressed protein on the virion surface and is critical for attachment to host cells and catalysis of membrane fusion. Ebolavirus GPs are described in, e.g., Lee and Saphire, 2009, Future Virol. 4(6):621-635 and Khataby et al., 2016, chapter in Ebola, doi:10.5772/64032.

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. The following strains of human coronaviruses are currently known: Human coronavirus 229E (HCoV-229E); Human coronavirus OC43 (HCoV-OC43); Severe acute respiratory syndrome coronavirus (SARS-CoV or SARS-CoV-1); Human coronavirus NL63 (HCoV-NL63, New Haven coronavirus); Human coronavirus HKU1 (HCoV-HKU1); Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019.” Multiple variants and subvariants of SARS-CoV-2 have been identified, including the Alpha, Beta, Gamma, Delta, and Omicron strains (see Garcia-Beltran, et al., 2021, medRxiv preprint Serv. Heal. Sci., doi: 10.1101/2021.02.14.21251704 and Alkhatib et al., 2021, Microbiology Spectrum 9(3)), all of which have mutations relative to the original SARS-CoV-2 isolate (see list of shared and unique mutations at covariants.org/shared-mutations). The coronaviruses HCoV-229E, -NL63, -OC43, and -HKU1 continually circulate in the human population and cause respiratory infections in adults and children world-wide.

Spike protein (or “S protein”) is a coronavirus surface protein that is able to mediate receptor binding and membrane fusion between a coronavirus virion and its host cell. Characteristic spikes on the surface of coronavirus virions are formed by ectodomains of homotrimers of Spike protein. In comparison to trimeric glycoproteins found on other human-pathogenic enveloped RNA viruses, coronavirus Spike protein is considerably larger, and totals nearly 450 kDa per trimer. Ectodomains of coronavirus Spike proteins contain an a N-terminal domain named S1, which is responsible for binding of receptors on the host cell surface, and a C-terminal S2 domain responsible for fusion. S1 domain of SARS-CoV-2 Spike protein is able to bind to Angiotensin-converting enzyme 2 (ACE2) of host cells. The region of SARS-CoV-2 Spike protein S1 domain that recognizes ACE2 is a 25 kDa domain called the receptor binding domain (RBD) (Walls et al., 2020, “Structure, Function, and antigenicity of the SARS-CoV-2 Spike Glycoprotein,” Cell 181(2):281-292.e6). Analysis of sera from COVID-19 patients demonstrates that antibodies are elicited against the Spike protein and can inhibit viral entry into the host cell (Brouwer et al., 2020, “Potent neutralizing antibodies from COVID-19 patients define multiple targets of vulnerability,” Science, 369(6504):643-650). The first Cryo-EM structure of SARS-CoV-2 Spike protein is described in Wrapp et al., 2020, “Cryo-EM structure of the 2019-nCoV spike in the prefusion conformation,” Science 367 (6483):1260-1263.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

A “domain” of a protein or a polypeptide refers to a region of the protein or polypeptide defined by structural and/or a functional properties. Exemplary function properties include enzymatic activity and/or the ability to bind to or be bound by another protein or non-protein entity.

The term “oligomer” and related terms, when used in reference to polypeptides or proteins, refer to complexes formed by two or more polypeptide or protein monomers, which can also be referred to as “subunits” or “chains.” For example, a trimer is an oligomer formed by three polypeptide subunits.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and 0-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The terms “fusion protein,” “fusion polypeptide,” and the related terms relate to polypeptide molecules, including artificial or engineered polypeptide molecules, that include two or more amino acid sequences previously found in separate polypeptide molecule, that are joined or linked in a fusion protein amino acid sequence to form a single polypeptide. For example, a fusion protein can be an engineered recombinant protein containing amino acid sequence from at least two unrelated proteins that have been joined together, via a peptide bond, to make a single protein. In this context, proteins are considered unrelated, if their amino acid sequences are not normally found joined together via a peptide bond in their natural environment, for example, inside a cell. The amino acid sequences of a fusion protein are encoded by corresponding nucleic acid sequences that are joined “in frame,” so that they are transcribed and translated to produce a single polypeptide. The amino acid sequences of a fusion protein can be contiguous or separated by one or more spacer, linker or hinge sequences. Fusion proteins can include additional amino acid sequences, such as, for example, signal sequences, tag sequences, and/or linker sequences.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Introduction

Provided in this disclosure are methods using machine learning to predict antibody variants that are likely to occur in nature. Such variants can be used (selected) to improve properties of the antibodies. The provided methods are described in detail in Section VIII, below. Also provided herein are antibodies and antigen binding portions thereof that specifically bind several antigens from coronaviruses, ebolaviruses, and influenza A viruses, various compositions of such antibodies or antigen binding portions thereof, recombinant nucleic acids encoding the antibodies and antigen binding portions thereof, and associated methods of use. As described in the Examples, embodiments of the viral antigen-specific antibodies and antigen binding portions thereof provided herein were designed using the provided methods.

An apparent paradox in evolutionary biology is how a random process can reliably generate new functions in short timescales across settings as diverse as antibody affinity maturation, viral immune escape, or tumor evolution [1]-[6]. The paradox arises from the difficulty of the task (exploring a combinatorially large space of possible sequences for rare mutations that improve fitness) contrasted with a seemingly simple set of tools (random mutation and recombination). Current approaches for directed evolution of proteins in the laboratory illustrate this contrast, as high-throughput evolutionary screens that rely on random guessing or brute-force search often devote substantial effort to interrogating weakly active or nonfunctional proteins.

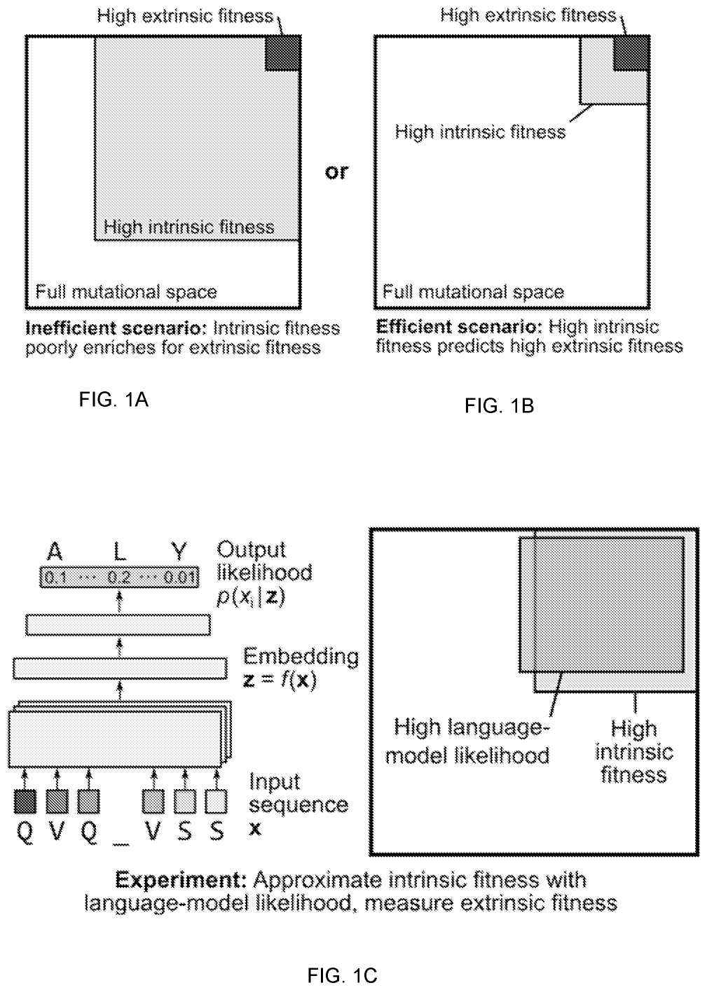

One approach to improve the efficiency of artificial evolution is to learn the rules of evolutionary plausibility (for example, sequences that result in a valid antibody) to help bias evolution away from invalid regimes (for example, mutations that cause an antibody to misfold) [7]. However, even if a search space were restricted to a set of evolutionarily plausible antibodies, the subset of those antibodies with improved binding affinity to a specific target might still be rare beyond practical utility (FIG. 1A). More broadly, a major open question is whether learning general evolutionary rules, or “intrinsic fitness,” is sufficient to enable efficient evolution under specific definitions of “extrinsic fitness” (for example, high binding affinity) [8].

As described herein and demonstrated in the Examples herein, evolutionary information alone can lead to improved fitness under specific selection pressures with high efficiency (FIG. 1B). The experimental test case described in the Examples focuses on affinity-maturation of human antibodies, in which high fitness is defined as stronger binding affinity to a particular antigen. Affinity maturation is a major application of directed evolution due to the therapeutic potential of antibodies with high affinity for disease targets [9]. Algorithms known as neural language models (FIG. 1C), which are trained on large datasets of sequences to learn patterns that are likely to occur in natural proteins [10]-[16], were used to model evolutionary plausibility. Notably, general language models were used that were trained on sequence datasets that are meant to represent variation across all observed natural proteins [17], rather than a language model that is restricted to variation among antibodies [18]-[21]. Given a single sequence, these language models were used to recommend evolutionarily-plausible mutations that we then experimentally screen for improved fitness. Importantly, the approach described herein was designed to be highly general. In some embodiments, the algorithm only requires a single wildtype sequence, without any initial binding affinity data, knowledge of the antigen, evolutionary homologs, or protein structure.

With this approach, six language models pretrained on ˜98 million natural sequences [17] were used to evolve seven human immunoglobulin G (IgG) antibodies that bind to antigens from coronavirus, ebolavirus, and influenza A virus. Viral antigens were the focus of the experiments described herein, given the importance of antibody-based therapeutics for epidemic and pandemic viral diseases [22]-[25]. When evolving highly-matured, clinically-relevant antibodies, the best design achieves a 10-fold improvement from wildtype; for unmatured antibodies, the best design achieves a 160-fold improvement. Many designs also showed preserved or improved thermostability and pseudovirus neutralization, including a significant improvement in the neutralization of a clinically-approved therapeutic antibody for Ebola. Notably, 20 or fewer new variants of each antibody across just two rounds of evolution were measured, which represents unprecedented efficiency for machine learning-guided directed evolution [26], [27] and which supports the practical utility of the approach described herein. This performance is especially striking because the models described herein have no initial task-specific training data.

As demonstrated herein, protein language models can guide highly efficient affinity maturation based on the wildtype antibody sequence alone, without requiring the researcher to supply any additional information on protein structure, binding specificity, or evolutionary homologs. Binding affinities were improved for a highly-evolved influenza A bnAb, MEDI8852, by up to 10-fold and a clinically-approved Ebola antibody, mAb114, by 3-fold. S309, a sarbecovirus bnAb, was also evolved to have higher affinity than a rationally-designed variant, sotrovimab, without lowering thermostability. Binding affinities were improved for unmatured antibodies from 13- to 160-fold across diverse antigens, which is within the 3.8- to 579-fold improvement range previously achieved by a state-of-the-art, in-vitro evolutionary system applied to unmatured, anti-RBD nanobodies [9]. Moreover, this was done with general protein language models that can also predict high-fitness variants across diverse protein families. This approach is envisioned to be useful in preclinical development as a rapid way to identify improved variants of an existing, antibody-of-interest (for example, a patient-derived antibody). Language models are also anticipated to become a key part of the antibody engineer's toolkit.

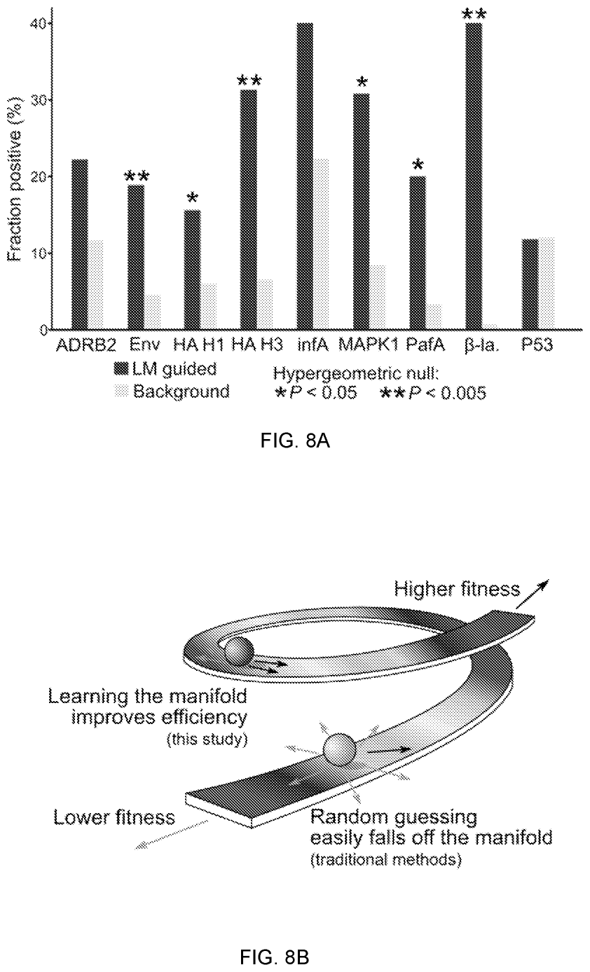

Beyond antibodies, this efficiency applies to other proteins as well. Because general protein language models that are trained across diverse protein families beyond antibodies were used, the same models that were used to affinity-mature antibodies can also predict antibiotic resistance, enzyme activity, or viral replication fitness. The results herein suggest that evolution guided by language models offers a compelling alternative to brute-force search, random guessing, or even rational design as a way to evolve proteins in the laboratory. Fundamentally, the results are surprising in that acquiring mutations simply based on evolutionary plausibility, or “intrinsic fitness,” sufficiently enriches for mutations that improve evolutionary specificity under natural selection pressures, or “extrinsic fitness” (FIG. 1B). Moreover, the success of the approach described herein challenges existing notions of evolutionary difficulty by suggesting that natural evolutionary manifolds are efficiently primed for fitness-enhancing mutations. Instead, in many settings, as long as evolution remains on a naturally plausible manifold, it is predicted that a substantial portion (greater than 10%) of mutations are bound to improve fitness, referred to herein as the “efficient manifold” hypothesis (FIG. 8B).

The efficient manifold hypothesis has direct, practical applications for those trying to evolve proteins in the laboratory. It is believed that evolution guided by a language model can be used as a drop-in replacement for current evolutionary tools based on randomization; for example, combinatorial libraries [39] can recombine language-model-guided mutations alongside or instead of rationally-chosen mutations [40]. By leveraging increasingly efficient technologies for nucleic acid printing [41], model-guided evolution could also directly replace mutagenesis strategies based on, for example, error-prone polymerases. To the end user, guiding evolution via pretrained, unsupervised models is also less resource-intensive than collecting enough task-specific data to train a supervised model [40]. The techniques described herein can also be used in conjunction with supervised approaches [7], [26], [27], [42]-[44], and supervising a model over multiple experimental rounds might ultimately lead to higher fitness, in many practical settings (for example, the rapid development of sotrovimab in response to the COVID-19 pandemic [28]), the efficiency of an unsupervised, single-round approach is preferable to a protracted, multi-round (machine-learning-guided) directed evolution campaign.

### II. Antibodies

In one aspect, the present disclosure provides antibodies and antigen binding portions thereof that bind specifically to one of several viral antigens. In some embodiments, the viral antigens are antigens from human coronaviruses, ebolaviruses, or influenza A viruses. In some embodiments, the human coronavirus antigen is aspike protein. In some embodiments, the ebolavirus antigen is an ebolavirus glycoprotein. In some embodiments, the influenza A antigen is a hemagglutinin (HA). The viral antigen antibodies and antigen binding portions thereof are polypeptides. As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. As used herein, the term antibody also encompasses an antibody fragment, for example, an antigen binding fragment. Antigen binding fragments comprise at least one antigen binding domain. One example of an antigen binding domain is an antigen binding domain formed by a VH-VL dimer. Antibodies and antigen binding fragments can be described by the antigen to which they specifically bind.

The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a R-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. Each VH and VL generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. (See Kabat et al. (1991) Sequences of Proteins of Immunological Interest 5th ed., Public Health Service, National Institutes of Health, Bethesda, MD.) CDR sequences on the heavy chain (VH) may be designated as CDRH1, 2, 3, while CDR sequences on the light chain (VL) may be designated as CDRL1, 2, 3. The amino acid sequences of the CDRs and framework regions can be determined using various well known definitions in the art, e.g., Kabat, Chothia, international ImMunoGeneTics database (IMGT), AbM, and observed antigen contacts (“Contact”). In some embodiments, CDRs are determined according to the IMGT definition. See, Brochet et al., 2008, Nucl. Acids Rex. 36:W503-508. In some embodiments, CDRs are determined by a combination of Kabat, Chothia, and/or Contact CDR definitions.

As used herein, the terms binds specifically to, specific for, binds selectively to and selective for a viral antigen (e.g., coronavirus spike protein, ebolavirus glycoprotein, or influenza A HA) or an epitope on a viral antigen mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target In that case, specific binding is indicated if the binding of the labeled target to a probe is competitively inhibited by the excess non-labeled target.

In each case, where a specific amino acid sequence is recited, embodiments comprising a sequence having at least 90% (e.g. 90%, 91%, 92%, 93%, 94%, 95%, 96%, 97%, 98%, or 99%) identity to the recited sequence are also provided.

As with all peptides, polypeptides, and proteins, including fragments thereof, it is understood that additional modifications in the amino acid sequence of the viral antigen-specific antibodies or antigen binding fragments thereof described herein, for example, in the heavy chain variable region and/or light chain variable region, can occur that do not alter the nature or function of the antibodies or antigen binding fragments thereof. Such modifications include conservative amino acids substitutions, such that each recited sequence optionally contains one or more conservative amino acid substitutions. The list provided below identifies groups that contain amino acids that are conservative substitutions for one another; these groups are exemplary as other conservative substitutions are known to those of skill in the art:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an aspartic acid at a specific residue is mentioned, also contemplated is a conservative substitution at the residue, for example, glutamic acid. Non-conservative substitutions, for example, substituting a proline with glycine or substituting a lysine with an asparagine, are also contemplated.

In some instances, the affinity of viral antigen-specific antibodies or antigen binding fragments thereof may be optimized through mutations to increase or decrease affinity as desired based on one or more of the known characteristics of the binding interaction with a viral antigen target, the structure of either or both of the antibodies or fragments thereof, or the viral antigen protein. In some instances, the mutations permit facile elution of purified antibodies or fragments thereof under desirable elution conditions during isolation and purification.

Methods of evaluating antibodies and antigen binding fragments thereof as provided in this disclosure are described in the Examples and are well-known in the art. Methods of further modifying antibodies for enhanced properties (e.g., enhanced affinity, chimerization, humanization) as well as generating antigen binding fragments, as described herein, are also well-known in the art.

In some embodiments, the heavy chain variable region and/or the light chain variable region of the monoclonal antibody has an identical sequence to the heavy chain variable region and/or the light chain variable region of the antibody produced by the methods described herein and in the Examples below. In some embodiments, the heavy chain variable region and/or the light chain variable region of the monoclonal antibody comprises one or more modifications, e.g., amino acid substitutions, deletions, or insertions.

The heavy chain variable region sequence and/or light chain variable region sequence of an antibody described herein can be engineered to comprise one or more variations in the heavy chain variable region sequence and/or light chain variable region sequence. In some embodiments, the engineered variation(s) improves the binding affinity of the antibody for a viral antigen target. In some embodiments, the engineered variation(s) improves the cross-reactivity of the antibody for a second antigen.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., PLoS Negl Trop Dis, 2017, 11:e0005655, doi:10.1371/journal.pntd.0005655.

In some embodiments, affinity maturation is used to engineer further mutations that enhance the binding affinity of the antibody for a viral antigen or enhance the cross-reactivity of the antibody for a second antigen. Methods for performing affinity maturation are known in the art. See, e.g., Renaut et al., Methods Mol Biol, 2012, 907:451-461.

The present disclosure also encompasses antibodies or fragments thereof that bind to the same epitope of a viral antigen (e.g., coronavirus spike protein, ebolavirus glycoprotein, or influenza A HA) as the antibodies disclosed herein. Such antibodies can be identified using routine techniques known in the art, including, for example, competitive binding assays.

The term epitope, as used herein, means a component of an antigen capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

The present disclosure also provides chimeric antibodies. The term chimeric antibody refers to an antibody in which a component of the heavy and/or light chain is derived from a particular source or species, while the remainder of the heavy and/or light chain is derived from a different source or species.

A human antibody is one that possesses an amino acid sequence corresponding to that of an antibody produced by a human or a human cell, or derived from a non-human source that utilizes a human antibody repertoire or human antibody-encoding sequences (e.g., obtained from human sources, genetically modified non-human sources or designed de novo). Human antibodies specifically exclude humanized antibodies.

Humanized forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies can also comprise residues that are not found in either the recipient antibody or the donor antibody. Such modifications can be made to further refine antibody function. (See Jones et al. (1986) Nature, 321:522-525; Riechmann et al. (1988) Nature, 332:323-329; and Presta, (1992) Curr Op Struct Biol., 2:593-596).

In some embodiments, the antibody or antigen binding fragment thereof provided herein can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to as a half antibody). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv, for example), single variable domain antibodies, diabodies (Dab) (bivalent and bispecific), and chimeric (e.g., humanized) antibodies, which may be produced by the modification of whole antibodies or synthesized de novo using recombinant DNA technologies. These functional antibody fragments retain the ability to bind specifically to their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains.

As used herein, the term monoclonal antibody refers to an antibody from a population of substantially homogeneous antibodies. A population of substantially homogeneous antibodies comprises antibodies that are the same or substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target, for example, by affinity maturation, to humanize the antibody, to improve its production in cell culture, and/or to reduce its immunogenicity in a subject.

Antigen binding fragments of an antibody molecule are well known in the art, and include, for example, (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv) (see e.g., Bird et al. (1988) Science 242:423-426; Huston et al. (1988) Proc. Natl. Acad. Sci. USA 85:5879-5883); (viii) a single domain antibody. These antibody fragments are obtained using conventional techniques known to those skilled in the art, and the fragments are screened for utility in the same manner as are intact antibodies.

In certain embodiments, antibodies and antibody compositions as provided herein are distinguishable from naturally occurring antibodies and compositions in one or more respects. Such distinguishable antibodies and compositions may be referred to as “synthetic,” or may be identified by the proviso that the antibody or composition “is not naturally occurring” or affirmatively as “non-naturally occurring.” As used herein the terms “corresponding antibody,” and “corresponding to” describes the relationship between (1) an antibody characterized by six specific CDR sequences and produced by the methods described herein and in the Examples below and (2) a synthetic antibody comprising the same six CDR sequences. Synthetic antibodies of this disclosure may differ in structure from naturally occurring antibodies with the same CDRs. That is, synthetic antibodies identified by specified CDRs may be structurally different from antibodies comprising the specified CDRs that are produced by the methods described herein and in the Examples below. Possible differences for synthetic antibodies include variable region sequences that differ from those of corresponding naturally occurring antibodies, different light chain sequences (i.e. lambda type instead of kappa type or vice versa), different isotypes, different allotypes, and different constant domain variants. These differences are discussed in more detail below.

In one approach, an antibody heavy chain comprises the CDRs of a clone described herein with the proviso that the antibody heavy chain does not comprise the heavy chain variable region sequence associated with the clone described herein. In some instances, both the heavy chain and the light chain variable region of an antibody have an amino acid sequence other than the sequence disclosed herein.

In some embodiments the synthetic antibody with specified CDRs is an isotype other than the isotype(s) found associated with the antibodies produced by the methods described herein and in the Examples below. In some embodiments the antibody disclosed herein is an isotype other than IgG1. In some embodiments the antibody disclosed herein is an isotype other than IgG2. In some embodiments the antibody disclosed herein is an isotype other than IgG3. In some embodiments the antibody disclosed herein is an isotype other than IgG4. In some embodiments the antibody disclosed herein is an isotype other than IgM. In some embodiments the antibody disclosed herein is an isotype other than IgA. In some embodiments the synthetic antibody comprises lambda type light chains. In some embodiments the synthetic antibody comprises kappa type light chains.

In some embodiments, the monoclonal antibody comprises a heavy chain variable region sequence and a light chain variable region sequence that are derived from an immunoglobulin producing human B cell, and further comprises a kappa or lambda light chain constant region. In some embodiments, the light chain constant region (kappa or lambda) is from the same type of light chain (i.e., kappa or lambda) as the light chain variable region that was derived from the immunoglobulin producing human B cell; as a non-limiting example, if an IgE-producing human B cell comprises a kappa light chain, then the monoclonal antibody that is produced can comprise the light chain variable region from the IgE-producing B cell and further comprises a kappa light chain constant region.

In some embodiments, the monoclonal antibody comprises a heavy chain variable region sequence and a light chain variable region sequence that are derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the monoclonal antibody comprises a native (i.e., wild-type) human IgG, IgA, IgM, or IgD constant region. In some embodiments, the monoclonal antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the monoclonal antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., J Biol Chem, 2015, 280:5462-5469.

In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. In some embodiments the monoclonal antibody comprises a human IgM constant region, human IgD constant region, human IgG constant region that is derived from IgG1, IgG2, IgG3, or IgG4, or human IgA constant region that is derived from IgA1 or IgA2. In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten or more modifications (e.g., amino acid substitutions). In some embodiments the constant regions includes variations (e.g., one, two, three, four, five, six, seven, eight, nine, ten or more amino acid substitutions) that reduce effector function.

In some embodiments the synthetic antibody with specified CDRs is an allotype other the allotype(s) found associated with the antibodies produced by the methods described herein and in the Examples below. The synthetic antibody may comprise an allotype selected from those listed in Table 4, below, which is different from an allotype of antibodies produced by the methods described herein and in the Examples below. In some embodiments, the synthetic antibody may comprise any individual allotype selected from those listed in Table 4, with the proviso that the allotype differs from the corresponding allotype of antibodies produced by the methods described herein and in the Examples below.

TABLE 1
Human immunoglobulin allotypes.

| | Heavy chains | | | | |
|---|---|---|---|---|---|
| Isotype/type | IgG1 | IgG2 | IgG3 | IgA | Light chains |
| Allotypes | G1m | G2m | G3m | A2m | Km |
| | 1(a) | 23(n) | 21(g1) | 1 | 1 |
| | 2(x) | | 28(g5) | 2 | 2 |
| | 3(f) | | 11(b0) | | 3 |
| | 17(z) | | 5(b1) | | |
| | | | 13 (b3) | | |
| | | | 14 (b4) | | |
| | | | 10 (b5) | | |
| | | | 15(s) | | |
| | | | 16(t) | | |
| | | | 6(c3) | | |
| | | | 24(c5) | | |
| | | | 26(u) | | |
| | | | 27 (v) | | |

NB: Alphabetical notation given within brackets. From: Jefferis and Marie-Paule Lefranc, 2009, “Human immunoglobulin allotypes: Possible implications for immunogenicity” mAbs 1(4): 332-338, incorporated herein by reference.

In some embodiments, a monoclonal antibody comprises CDR sequences, a heavy chain variable region, and/or a light chain variable region as described herein (e.g., as disclosed in Table 1, Table 2, and Table 3) and further comprises a heavy chain constant region and/or a light chain constant region that is heterologous to the antibody produced by the methods described herein and in the Examples below from which the CDR sequences and/or variable region sequences are derived. For example, in some embodiments, the monoclonal antibody comprises the CDR sequences and/or variable region sequences of an antibody produced by the methods described herein and in the Examples below, and further comprises a heavy chain constant region and a light chain constant region that is heterologous to the antibody produced by the methods described herein and in the Examples below (e.g., the heavy chain constant region and/or light chain constant region is a wild-type or modified IgG1, IgG2, IgG3, or IgG4 constant region), or the heavy chain constant region and/or light chain constant region comprises one or more modifications (e.g., amino acid substitutions) relative to the native constant region of the antibodies produced by the methods described herein and in the Examples below.

Synthetic antibodies of this disclosure may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-like, modulate coengagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fc engineering to modulate antibody effector functions” Protein Cell 2018, 9(1):63-73; Schlothauer, 2016, “Novel human IgG1 and IgG4 Fc-engineered antibodies with completely abolished immune effector functions,” Protein Engineering, Design and Selection 29(10):457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca and guanaco) or other species besides Camelidae.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is screened, and in accordance with the methods described herein, against a target antigen of interest to identify those variants having optimal selectivity and affinity for the target antigen. See, e.g., Hey et al. (2005) TRENDS Biotechnol 23(10):514-522.

One of skill in the art would appreciate that the scaffold portion of the non-antibody scaffold protein can include, e.g., all or part of the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins, such as those described in, e.g., International Application Publication No. WO2015/104406), human crystallin, human ubiquitin, or a trypsin inhibitor from E. elaterium.

Synthetic antibody compositions of this disclosure may differ from naturally occurring compositions in at least one or more of the following respects: (i) composition comprises antibodies that are purified, i.e., separated from tissue or cellular material with which they are associated in the human body, and optionally in a manufactured excipient or medium; and/or (ii) antibody compositions of this disclosure contain a single species of antibody (are monoclonal) such that all antibodies in the composition have the same structure and specificity.

Any of the viral antigen-specific antibodies or antigen binding fragments thereof described herein can be modified with covalent and/or non-covalent modifications. Such modifications can be introduced into the antibodies or antigen binding fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments. Recombinant techniques can be used to modify antibodies or antigen binding fragments thereof. For example, amino acids found to not contribute to either the activity or the binding specificity or affinity of the antibody can be deleted without a loss in the respective activity. Insertions, deletions, substitutions, or other selected modifications of particular regions or specific amino acids residues, provided the activity of the fragment is not significantly altered or impaired compared to the non-modified antibody, or antigen binding fragment thereof can be made. Such methods are readily apparent to a skilled practitioner in the art and can include site specific mutagenesis of the nucleic acid encoding the antibody or fragment thereof. (Zoller et al., Nucl. Acids Res. 10:6487-500 (1982)). In some instances, the viral antigen-specific antibodies or antigen binding fragments may be labeled by a variety of means for use in diagnostic and/or pharmaceutical applications.

In some embodiments, the antibodies or antigen binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. In some embodiments, the heterologous moiety is an antibody or antigen binding fragment thereof that specifically binds to a different target, and such a conjugated antibody is referred to as a bispecific antibody. Additional suitable heterologous polypeptides include, e.g., an antigenic tag (e.g., FLAG (DYKDDDDK) (SEQ ID NO:55), polyhistidine (6-His; HHHHHH (SEQ ID NO:56)), hemagglutinin (HA; YPYDVPDYA (SEQ ID NO:57)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase. Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific antihapten antibodies.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

Techniques for conjugating a therapeutic moiety to a viral antigen-specific antibody or antigen binding fragment thereof as described herein are well known, see, for example, Amon et al., Monoclonal Antibodies And Cancer Therapy, Reisfeld et al. (eds.), pp. 243-56 (1985); Hellstrom et al., Controlled Drug Delivery (2nd Ed.), Robinson et al. (eds.), pp. 623-53 (1987); Thorpe, Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy” In: Monoclonal Antibodies For Cancer Detection And Therapy, (Baldwin et al. eds.), pp. 303-316 (1985), and Thorpe et al., Immunol. Rev. 62:119-158 (1982). Alternatively, an antibody can be conjugated to a second antibody to form an antibody heteroconjugate (e.g., a bispecific antibody) as described in U.S. Pat. No. 4,676,980.

In some embodiments, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵I]mIPNHS), which binds to free amino groups to form meta-iodophenyl (mIP) derivatives of relevant proteins (see, e.g., Rogers et al. (1997) J Nucl Med 38:1221-1229) or chelate (e.g., to DOTA or DTPA), which is in turn bound to the protein backbone. Methods of conjugating the radioactive labels or larger molecules/chelates containing them to the antibodies or antigen binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisid et al. (2010) Int J Pharm 387(1-2):110-119). The stabilization moiety can improve the stability, or retention of, the antibody (or fragment) by at least 1.5 (e.g., at least 2, 5, 10, 15, 20, 25, 30, 40, or 50 or more) fold.

TABLE 2
Antibody VH and VL amino acid sequences of known antibodies.

| Antibody | | |
|---|---|---|
| ID | VH sequence | VL sequence |
| MEDI8852 | SEQ ID NO: 1 | SEQ ID NO: 2 |
| | QVQLQQSGPGLVKPSQTLSLTCAISGDSVSSYNAVWNWI | DIQMTQSPSSLSASVGDRVTITCRTSQSLSSYTHWYQQKPGKAPKLLIY |
| | RQSPSRGLEWLGRTYYRSGWYNDYAESVKSRITINPDTS | AASSRGSGVPSRFSGSGSGTDFTLTISSLQPEDFATYYCQQSRTFGQGTK |
| | KNQFSLQLNSVTPEDTAVYYCARSGHITVFGVNVDAFDM | VEIK |
| | WGQGTMVTVSS | |
| MEDI8852 | SEQ ID NO: 3 | SEQ ID NO: 4 |
| UCA | QVQLQQSGPGLVKPSQTLSLTCAISGDSVSSNSAAWNWI | DIQMTQSPSSLSASVGDRVTITCRASQSISSYLNWYQQKPGKAPKLLIY |
| | RQSPSRGLEWLGRTYYRSKWYNDYAVSVKSRITINPDTS | AASSLQSGVPSRFSGSGSGTDFTLTISSLQPEDFATYYCQQSRTFGQGTK |
| | KNQFSLQLNSVTPEDTAVYYCARGGHITIFGVNIDAFDI | VEIK |
| | WGQGTMVTVSS | |
| mAb114 | SEQ ID NO: 5 | SEQ ID NO: 6 |
| | EVQLVESGGGLIQPGGSLRLSCAASGFALRMYDMHWVRQ | DIQMTQSPSSLSASVGDRITITCRASQAFDNYVAWYQQRPGKVPKLLIS |
| | TIDKRLEWVSAVGPSGDTYYADSVKGRFAVSRENAKNSL | AASALHAGVPSRFSGSGSGTHFTLTISSLQPEDVATYYCQNYNSAPLTF |
| | SLQMNSLTAGDTAIYYCVRSDRGVAGLFDSWGQGILVTV | GGGTKVEIK |
| | SS | |
| mAb114 | SEQ ID NO: 7 | SEQ ID NO: 8 |
| UCA | EVQLVESGGGLVQPGGSLRLSCAASGFTFSSYDMHWVRQ | DIQMTQSPSSLSASVGDRVTITCRASQGISNYLAWYQQKPGKVPKLLIY |
| | ATGKGLEWVSAIGTAGDTYYPGSVKGRFTISRENAKNSL | AASTLQSGVPSRFSGSGSGTDFTLTISSLQPEDVATYYCQKYNSAPLTF |
| | YLQMNSLRAGDTAVYYCVRSDRGVAGLFDSWGQGTLVTV | GGGTKVEIK |
| | SS | |
| S309 | SEQ ID NO: 9 | SEQ ID NO: 10 |
| | QVQLVQSGAEVKKPGASVKVSCKASGYPFTSYGISWVRQ | EIVLTQSPGTLSLSPGERATLSCRASQTVSSTSLAWYQQKPGQAPRLLIY |
| | APGQGLEWMGWISTYNGNTNYAQKFQGRVTMTTDTSTTT | GASSRATGIPDRFSGSGSGTDFTLTISRLEPEDFAVYYCQQHDTSLTFG |
| | GYMELRRLRSDDTAVYYCARDYTRGAWFGESLIGGEDNW | GGTK VEIK |
| | GQGTLVTVSS | |
| REGN10987 | SEQ ID NO: 11 | SEQ ID NO: 12 |
| | QVQLVESGGGVVQPGRSLRLSCAASGFTFSNYAMYWVRQ | QSALTQPASVSGSPGQSITISCTGTSSDVGGYNYVSWYQQHPGKAPKL |
| | APGKGLEWVAVISYDGSNKYYADSVKGRFTISRDNSKNT | MIYDVSKRPSGVSNRFSGSKSGNTASLTISGLQSEDEADYYCNSLTSIST |
| | LYLQMNSLRTEDTAVYYCASGSDYGDYLLVYWGQGTLVT | WVFGGGTKLTVL |
| | VSS | |
| C143 | SEQ ID NO: 13 | SEQ ID NO: 14 |
| | EVQLVESGGGLVQPGGSLRLSCAASGFSVSTKYMTWVRQ | QSALTQPASVSGSPGQSITISCTGTSNDVGSYTLVSWYQQYPGKAPKLL |
| | APGKGLEWVSVLYSGGSDYYADSVKGRFTISRDNSKNAL | IFEGTKRSSGISNRFSGSKSGNTASLTISGLQGEDEADYYCCSYAGASTF |
| | YLQMNSLRVEDTGVYYCARDSSEVRDHPGHPGRSVGAFD | VFGGGTKLTVL |
| | IWGQGTMVTVSS | |
| LY-1404 | SEQ ID NO: 58 | SEQ ID NO: 59 |
| | QITLKESGPTLVKPTQTLTLTCTFSGFSLSISGVGVGWL | QSALTQPASVSGSPGQSITISCTATSSDVGDYNYVSWYQQHPGKAPKL |
| | RQPPGKALEWLALIYWDDDKRYSPSLKSRLTISKDTSKN | MIFEVSDRPSGISNRFSGSKSGNTASLTISGLQAEDEADYYCSSYTTSSA |
| | QVVLKMTNIDPVDTATYYCAHHSISTIFDHWGQGTLVTV | VFGGGTKLTVL |
| | SS | |
| SA58 | SEQ ID NO: 60 | SEQ ID NO: 61 |
| | QVQLAQSGSELRKPGASVKVSCDTSGHSFTSNAIHWVRQ | EVVMTQSPASLSVSPGERATLSCRARASLGISTDLAWYQQRPGQAPRL |
| | APGQGLEWMGWINTDTGTPTYAQGFTGRFVFSLDTSART | LIYGASTRATGIPARFSGSGSGTEFTLTISSLQSEDSAVYYCQQYSNWPL |
| | AYLQISSLKADDTAVFYCARERDYSDYFFDYWGQGTLVT | TFGGGTKVEIK |
| | VSS | |

TABLE 3
Heavy chain variable domain (VH) CDR amino acid sequences of known antibodies.

| Antibody | | | | |
|---|---|---|---|---|
| ID | VH sequence | CDR1 | CDR2 | CDR3 |
| MEDI8852 | SEQ ID NO: 1 | SYNAVWN | RTYYRSGWYNDYAESVKS | SGHITVFGVNVDAFDM |
| | | (SEQ ID NO: 15) | (SEQ ID NO: 16) | (SEQ ID NO: 17) |
| MEDI8852 | SEQ ID NO: 3 | SNSAAWN | RTYYRSKWYNDYAVSVKS | GGHITIFGVNIDAFDI |
| UCA | | (SEQ ID NO: 18) | (SEQ ID NO: 19) | (SEQ ID NO: 20) |
| mAb114 | SEQ ID NO: 5 | MYDMH | AVGPSGDTYYADSVKG | SDRGVAGLFDS |
| | | (SEQ ID NO: 21) | (SEQ ID NO: 22) | (SEQ ID NO: 23) |
| mAb114 | SEQ ID NO: 7 | SYDMH | AIGTAGDTYYPGSVKG | SDRGVAGLFDS |
| UCA | | (SEQ ID NO: 24) | (SEQ ID NO: 25) | (SEQ ID NO: 23) |
| S309 | SEQ ID NO: 9 | SYGIS | WISTYNGNTNYAQKFQG | DYTRGAWFGESLIGGFDN |
| | | (SEQ ID NO: 26) | (SEQ ID NO: 27) | (SEQ ID NO: 28) |
| REGN10987 | SEQ ID NO: 11 | NYAMY | VISYDGSNKYYADSVKG | GSDYGDYLLVY |
| | | (SEQ ID NO: 29) | (SEQ ID NO: 30) | (SEQ ID NO: 31) |
| C143 | SEQ ID NO: 13 | TKYMT | VLYSGGSDYYADSVKG | DSSEVRDHPGHPGRSVGAFDI |
| | | (SEQ ID NO: 32) | (SEQ ID NO: 33) | (SEQ ID NO: 34) |
| LY-1404 | SEQ ID NO: 58 | ISGVGVG | LIYWDDDKRYSPSLKS | HSISTIFDH |
| | | (SEQ ID NO: 62) | (SEQ ID NO: 63) | (SEQ ID NO: 64) |
| SA58 | SEQ ID NO: 60 | SNAIH | WINTDTGTPTYAQGFTG | ERDYSDYFFD |
| | | (SEQ ID NO: 65) | (SEQ ID NO: 66) | (SEQ ID NO: 67) |

TABLE 4
Light chain variable domain (VL) CDR amino acid
sequences of known antibodies.

| Antibody | | | | |
|---|---|---|---|---|
| ID | VL sequence | CDR1 | CDR2 | CDR3 |
| MEDI8852 | SEQ ID NO: 2 | RTSQSLSSYTH | AASSRGS | QQSRT |
| | | (SEQ ID NO: 35) | (SEQ ID NO: 36) | (SEQ ID NO: 37) |
| MEDI8852 | SEQ ID NO: 4 | RASQSISSYLN | AASSLQS | QQSRT |
| UCA | | (SEQ ID NO: 38) | (SEQ ID NO: 39) | (SEQ ID NO: 37) |
| mAb114 | SEQ ID NO: 6 | RASQAFDNYVA | AASALHA | QNYNSAPLT |
| | | (SEQ ID NO: 40) | (SEQ ID NO: 41) | (SEQ ID NO: 42) |
| mAb114 | SEQ ID NO: 8 | RASQGISNYLA | AASTLQS | QKYNSAPLT |
| UCA | | (SEQ ID NO: 43) | (SEQ ID NO: 44) | (SEQ ID NO: 45) |
| S309 | SEQ ID NO: 10 | RASQTVSSTSLA | GASSRAT | QQHDTSLT |
| | | (SEQ ID NO: 46) | (SEQ ID NO: 47) | (SEQ ID NO: 48) |
| REGN10987 | SEQ ID NO: 12 | TGTSSDVGGYNYVS | DVSKRPS | NSLTSISTWV |
| | | (SEQ ID NO: 49) | (SEQ ID NO: 50) | (SEQ ID NO: 51) |
| C143 | SEQ ID NO: 14 | TGTSNDVGSYTLVS | EGTKRSS | CSYAGASTFV |
| | | (SEQ ID NO: 52) | (SEQ ID NO: 53) | (SEQ ID NO: 54) |
| LY-1404 | SEQ ID NO: 59 | TATSSDVGDYNYVS | EVSDRPS | SSYTTSSAV |
| | | (SEQ ID NO: 68) | (SEQ ID NO: 69) | (SEQ ID NO: 70) |
| SA58 | SEQ ID NO: 61 | RARASLGISTDLA | GASTRAT | QQYSNWPLT |
| | | (SEQ ID NO: 71) | (SEQ ID NO: 72) | (SEQ ID NO: 73) |

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### Exemplary Embodiments of Antibodies and Antigen-Binding Portions Thereof.

Provided herein are antibodies or antigen binding portions thereof that bind specifically to viral antigens (e.g., coronavirus spike protein, ebolavirus glycoprotein, or influenza A HA). Viral antigen-specific antibodies were identified and tested as described herein and in the Examples below. In some embodiments, the antibodies or antigen binding portions thereof provided herein are derived from known antibodies (e.g., according to the methods described in Section VIII and the Examples herein). The known antibodies are described in more detail in Example 2 herein. The heavy chain variable region sequences and light chain variable region sequences of the known antibodies encompassed by this disclosure are set forth in Table 1. The predicted heavy chain CDR sequences of the known antibodies are set forth in Table 2. The predicted light chain CDR sequences of the known antibodies are set forth in Table 3. In Table 1, the CDR sequences in the variable domains are indicated by bold and underlined text. In some embodiments, the antibodies or antigen binding portions thereof provided herein are described in reference to the known antibody sequences as set forth in Table 1, Table 2, and Table 3. For example, many of the provided antibodies or antigen-binding portions thereof comprise one or more amino acid residue substitutions in a heavy chain and/or light chain variable region relative to the amino acid sequences set forth in Table 1. Exemplary embodiments of the antibodies and antigen-binding portions thereof provided herein are described below and in the Examples herein.

1. Antibodies and Antigen-Binding Portions Thereof Derived from the MEDI8852 Antibody.

In one aspect, provided herein are antibodies and antigen-binding portions thereof that specifically bind to influenza A hemagglutinin. In some embodiments, such antibodies and antigen-binding portions thereof are derived from the MEDI8852 broadly-neutralizing antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:15, a CDRH2 comprising at least 70% identity to SEQ ID NO: 16, and a CDRH3 comprising at least 70% identity to SEQ ID NO:17 and/or a light chain variable region comprising a CDRL1 comprising at least 70% identity to SEQ ID NO:35, a CDRL2 comprising at least 70% identity to SEQ ID NO:36, and a CDRL3 comprising at least 70% identity to SEQ ID NO:37. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:1 and/or a light chain variable region comprising at least 70% identity to SEQ ID NO:2.

In some embodiments, the heavy chain variable region comprises an amino acid residue substitution at at least one (e.g., one, two, three, four, or more) of positions 124, D27, S44, T53, E65, N74, P75, or M117, wherein the positions are numbered with respect to SEQ ID NO: 1. In some embodiments, the light chain variable region comprises an amino acid residue substitution at at least one (e.g., one, two, three, four, or more) of positions T25, L29, T33, G55, R92, and G95, wherein the positions are numbered with respect to SEQ ID NO:2. In some embodiments, the heavy chain variable region comprises amino acid substitutions at any of the residue positions or combinations of residue positions represented in Table 6, below. In some embodiments, the light chain variable region comprises amino acid substitutions at any of the residue positions or combinations of residue positions represented in Table 6, below. In some embodiments, both the heavy chain variable region comprises any of the the heavy chain variable region amino acid substitutions or combinations of amino acid substitutions at any of the residue positions or combinations of residue positions represented in Table 6, below, and the light chain variable region comprises any of the amino acid substitutions or combinations of the light chain variable region amino acid substitutions at any of the residue positions or combinations of residue positions represented in Table 6, below.

In some embodiments, the heavy chain variable region comprises at least one of the amino acid residue substitutions I24V, D27F, S44G, T53I, E65P, E65R, N74S, P75R, M117Y, or any combination thereof wherein the positions are numbered with respect to SEQ ID NO: 1. In some embodiments, the heavy chain variable region comprises two, three, four, five, six, seven, eight, or nine of the above substitutions. In some embodiments, the light chain variable region comprises at least one of the amino acid residue substitutions T25A, L29V, T33L, G55A, R92D, G95P, or any combination thereof wherein the positions are numbered with respect to SEQ ID NO:2. In some embodiments, the light chain variable region comprises two, three, four, five, or six of the above substitutions. In some embodiments of the antibody described in this section, both the light and the heavy chains comprise substitutions. In some embodiments, the heavy chain variable region comprises any of the amino acid substitutions or combinations of amino acid substitutions represented in Table 6, below. In some embodiments, the light chain variable region comprises any of the amino acid substitutions or combinations of amino acid substitutions represented in Table 6, below. In some embodiments, both the heavy chain variable region comprises any of the the heavy chain variable region amino acid substitutions or combinations of amino acid substitutions represented in Table 6, below, and the light chain variable region comprises any of the amino acid substitutions or combinations of the light chain variable region amino acid substitutions represented in Table 6, below.

2. Antibodies and Antigen-Binding Portions Thereof Derived from the MEDI8852 Unmutated Common Ancestor Antibody.

In some embodiments, antibodies and antigen-binding portions thereof that specifically bind to influenza A hemagglutinin are derived from the MEDI8852 unmutated common ancestor (UCA), as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 18, a CDRH2 comprising at least 70% identity to SEQ ID NO: 19, and a CDRH3 comprising at least 70% identity to SEQ ID NO:20 and/or a light chain variable region comprising a CDRL1 comprising at least 70% identity to SEQ ID NO:38, a CDRL2 comprising at least 70% identity to SEQ ID NO:39, and a CDRL3 comprising at least 70% identity to SEQ ID NO:37. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:3 and/or a light chain variable region comprising at least 70% identity to SEQ ID NO:4.

In some embodiments, the heavy chain variable region comprises an amino acid residue substitution at at least one (e.g., one, two, three, four, or more) of positions S44, T53, K58, V65, N74, and P75, wherein the positions are numbered with respect to SEQ ID NO:3. In some embodiments, the light chain variable region comprises an amino acid residue substitution at at least one (e.g., one or two) of positions N34 and G95, wherein the positions are numbered with respect to SEQ ID NO:4. In some embodiments, the heavy chain variable region comprises amino acid substitutions at any of the residue positions or combinations of residue positions represented in Table 8, below. In some embodiments, the light chain variable region comprises amino acid substitutions at any of the residue positions or combinations of residue positions represented in Table 8, below. In some embodiments, both the heavy chain variable region comprises any of the the heavy chain variable region amino acid substitutions or combinations of amino acid substitutions at any of the residue positions or combinations of residue positions represented in Table 8, below, and the light chain variable region comprises any of the amino acid substitutions or combinations of the light chain variable region amino acid substitutions at any of the residue positions or combinations of residue positions represented in Table 8, below.

In some embodiments, the heavy chain variable region comprises at least one of the amino acid residue substitutions S44G, T53I, K58S, V65P, N74S, P75R, or any combination thereof, wherein the positions are numbered with respect to SEQ ID NO:3. In some embodiments, the heavy chain variable region comprises two, three, four, five, or six of the above substitutions. In some embodiments, the light chain variable region comprises one or both of the amino acid residue substitutions N34A, G95P, wherein the positions are numbered with respect to SEQ ID NO:4. In some embodiments of the antibody described in this section, both the light and the heavy chains comprise substitutions. In some embodiments, the heavy chain variable region comprises any of the amino acid substitutions or combinations of amino acid substitutions represented in Table 8, below. In some embodiments, the light chain variable region comprises any of the amino acid substitutions or combinations of amino acid substitutions represented in Table 8, below. In some embodiments, both the heavy chain variable region comprises any of the the heavy chain variable region amino acid substitutions or combinations of amino acid substitutions represented in Table 8, below, and the light chain variable region comprises any of the amino acid substitutions or combinations of the light chain variable region amino acid substitutions represented in Table 8, below.

3. Antibodies and Antigen-Binding Portions Thereof Derived from the mAb114 Antibody.

In another aspect, provided herein are antibodies and antigen-binding portions thereof that specifically bind to ebolavirus glycoprotein. In some embodiments, such antibodies and antigen-binding portions thereof are derived from the mAb 114 patient-derived antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:21, a CDRH2 comprising at least 70% identity to SEQ ID NO:22, and a CDRH3 comprising at least 70% identity to SEQ ID NO:23 and/or a light chain variable region comprising a CDRL1 comprising at least 70% identity to SEQ ID NO:40, a CDRL2 comprising at least 70% identity to SEQ ID NO:41, and a CDRL3 comprising at least 70% identity to SEQ ID NO:42. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:5 and/or a light chain variable region comprising at least 70% identity to SEQ ID NO:6.

In some embodiments, the heavy chain variable region comprises an amino acid residue substitution at at least one (e.g., one, two, three, four, or more) of M31, 141, D42, A68, E72, S79, and I113, wherein the positions are numbered with respect to SEQ ID NO:5. In some embodiments, the light chain variable region comprises an amino acid residue substitution at at least one (e.g., one, two, three, four, or more) of positions 119, F29, V43, S49, H70, and N90, wherein the positions are numbered with respect to SEQ ID NO:6. In some embodiments, the heavy chain variable region comprises amino acid substitutions at any of the residue positions or combinations of residue positions represented in Table 9, below. In some embodiments, the light chain variable region comprises amino acid substitutions at any of the residue positions or combinations of residue positions represented in Table 9, below. In some embodiments, both the heavy chain variable region comprises any of the the heavy chain variable region amino acid substitutions or combinations of amino acid substitutions at any of the residue positions or combinations of residue positions represented in Table 9, below, and the light chain variable region comprises any of the amino acid substitutions or combinations of the light chain variable region amino acid substitutions at any of the residue positions or combinations of residue positions represented in Table 9, below.

In some embodiments, the heavy chain variable region comprises at least one of the amino acid residue substitutions M31S, I41P, D42G, A68T, E72D, S79Y, I113T, or any combination thereof, wherein the positions are numbered with respect to SEQ ID NO:5. In some embodiments, the heavy chain variable region comprises two, three, four, five, six, or seven of the above substitutions. In some embodiments, the light chain variable region comprises at least one of the amino acid residue substitutions I19V, F29I, V43A, S49Y, H70D, N90Q, or any combination thereof, wherein the positions are numbered with respect to SEQ ID NO:6. In some embodiments, the light chain variable region comprises two, three, four, five, or six of the above substitutions. In some embodiments of the antibody described in this section, both the light and the heavy chains comprise substitutions. In some embodiments, the light chain variable region comprises two, three, or four of the above substitutions. In some embodiments of the antibody described in this section, both the light and the heavy chains comprise substitutions. In some embodiments, the heavy chain variable region comprises any of the amino acid substitutions or combinations of amino acid substitutions represented in Table 9, below. In some embodiments, the light chain variable region comprises any of the amino acid substitutions or combinations of amino acid substitutions represented in Table 9, below. In some embodiments, both the heavy chain variable region comprises any of the the heavy chain variable region amino acid substitutions or combinations of amino acid substitutions represented in Table 9, below, and the light chain variable region comprises any of the amino acid substitutions or combinations of the light chain variable region amino acid substitutions represented in Table 9, below.

4. Antibodies and Antigen-Binding Portions Thereof Derived from the mAb114 Unmutated Common Ancestor Antibody.

In some embodiments, antibodies and antigen-binding portions thereof that specifically bind to ebolavirus glycoprotein are derived from the mAb 114 UCA, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:24, a CDRH2 comprising at least 70% identity to SEQ ID NO:25, and a CDRH3 comprising at least 70% identity to SEQ ID NO:23 and/or a light chain variable region comprising a CDRL1 comprising at least 70% identity to SEQ ID NO:43, a CDRL2 comprising at least 70% identity to SEQ ID NO:44, and a CDRL3 comprising at least 70% identity to SEQ ID NO:45. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:7 and/or a light chain variable region comprising at least 70% identity to SEQ ID NO: 8.

In some embodiments, the heavy chain variable region comprises an amino acid residue substitution at at least one (e.g., one, two, three, four, or more) of positions T41, A54, P60, G61, E72, G88, and V96, wherein the positions are numbered with respect to SEQ ID NO:7. In some embodiments, the light chain variable region comprises an amino acid residue substitution at at least one (e.g., one or two) of positions V43 and K90, wherein the positions are numbered with respect to SEQ ID NO:8. In some embodiments, the heavy chain variable region comprises amino acid substitutions at any of the residue positions or combinations of residue positions represented in Table 10, below. In some embodiments, the light chain variable region comprises amino acid substitutions at any of the residue positions or combinations of residue positions represented in Table 10, below. In some embodiments, both the heavy chain variable region comprises any of the the heavy chain variable region amino acid substitutions or combinations of amino acid substitutions at any of the residue positions or combinations of residue positions represented in Table 10, below, and the light chain variable region comprises any of the amino acid substitutions or combinations of the light chain variable region amino acid substitutions at any of the residue positions or combinations of residue positions represented in Table 10, below.

In some embodiments, the heavy chain variable region comprises at least one of the amino acid residue substitutions T41P, A54G, P60A, G61D, E72D, G88E, V96A, or any combination thereof, wherein the positions are numbered with respect to SEQ ID NO:7. In some embodiments, the heavy chain variable region comprises two, three, four, five, six, or seven of the above substitutions. In some embodiments, the light chain variable region comprises at least one or both of the amino acid residue substitutions V43A, K90Q, wherein the positions are numbered with respect to SEQ ID NO:8. In some embodiments of the antibody described in this section, both the light and the heavy chains comprise substitutions. In some embodiments, the heavy chain variable region comprises any of the amino acid substitutions or combinations of amino acid substitutions represented in Table 10, below. In some embodiments, the light chain variable region comprises any of the amino acid substitutions or combinations of amino acid substitutions represented in Table 10, below. In some embodiments, both the heavy chain variable region comprises any of the the heavy chain variable region amino acid substitutions or combinations of amino acid substitutions represented in Table 10, below, and the light chain variable region comprises any of the amino acid substitutions or combinations of the light chain variable region amino acid substitutions represented in Table 10, below.

5. Antibodies and Antigen-Binding Portions Thereof Derived from the S309 Antibody.

In another aspect, provided herein are antibodies and antigen-binding portions thereof that specifically bind to coronavirus spike protein (e.g., a SARS-CoV-1 spike protein or a SARS-CoV-2 spike protein). In some embodiments, such antibodies and antigen-binding portions thereof are derived from the S309 patient-derived antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:26, a CDRH2 comprising at least 70% identity to SEQ ID NO:27, and a CDRH3 comprising at least 70% identity to SEQ ID NO:28 and/or a light chain variable region comprising a CDRL1 comprising at least 70% identity to SEQ ID NO:46, a CDRL2 comprising at least 70% identity to SEQ ID NO:47, and a CDRL3 comprising at least 70% identity to SEQ ID NO:48. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:9 and/or a light chain variable region comprising at least 70% identity to SEQ ID NO: 10.

In some embodiments, the heavy chain variable region comprises an amino acid residue substitution at at least one (e.g., one, two, three, four, or more) of P28, T77, G79, R84, R85, and R87, wherein the positions are numbered with respect to SEQ ID NO:9. In some embodiments, the light chain variable region comprises an amino acid residue substitution at at least one (e.g., one, two, three, or four) of positions T28, T32, S95, and L96, wherein the positions are numbered with respect to SEQ ID NO: 10. In some embodiments, the heavy chain variable region comprises amino acid substitutions at any of the residue positions or combinations of residue positions represented in Table 11, below. In some embodiments, the light chain variable region comprises amino acid substitutions at any of the residue positions or combinations of residue positions represented in Table 11, below. In some embodiments, both the heavy chain variable region comprises any of the the heavy chain variable region amino acid substitutions or combinations of amino acid substitutions at any of the residue positions or combinations of residue positions represented in Table 11, below, and the light chain variable region comprises any of the amino acid substitutions or combinations of the light chain variable region amino acid substitutions at any of the residue positions or combinations of residue positions represented in Table 11, below.

In some embodiments, the heavy chain variable region comprises at least one of the amino acid residue substitutions P28T, T77N, G79A, R84S, R85S, R87T, or any combination thereof, wherein the positions are numbered with respect to SEQ ID NO:9. In some embodiments, the heavy chain variable region comprises two, three, four, five, or six of the above substitutions. In some embodiments, the light chain variable region comprises at least one of the amino acid residue T28S, T32S, S95V, L96P, or any combination thereof, wherein the positions are numbered with respect to SEQ ID NO: 10. In some embodiments, the light chain variable region comprises two, three, or four of the above substitutions. In some embodiments of the antibody described in this section, both the light and the heavy chains comprise substitutions. In some embodiments, the heavy chain variable region comprises any of the amino acid substitutions or combinations of amino acid substitutions represented in Table 11, below. In some embodiments, the light chain variable region comprises any of the amino acid substitutions or combinations of amino acid substitutions represented in Table 11, below. In some embodiments, both the heavy chain variable region comprises any of the the heavy chain variable region amino acid substitutions or combinations of amino acid substitutions represented in Table 11, below, and the light chain variable region comprises any of the amino acid substitutions or combinations of the light chain variable region amino acid substitutions represented in Table 11, below.

6. Antibodies and Antigen-Binding Portions Thereof Derived from the REGN10987 Antibody.

In some embodiments, the antibodies and antigen-binding portions thereof that specifically bind to coronavirus spike protein (e.g., a SARS-CoV-2 spike protein) are derived from the REGN10987 patient-derived antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:29, a CDRH2 comprising at least 70% identity to SEQ ID NO:30, and a CDRH3 comprising at least 70% identity to SEQ ID NO:31 and/or a light chain variable region comprising a CDRL1 comprising at least 70% identity to SEQ ID NO:49, a CDRL2 comprising at least 70% identity to SEQ ID NO:50, and a CDRL3 comprising at least 70% identity to SEQ ID NO:51. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 11 and/or a light chain variable region comprising at least 70% identity to SEQ ID NO: 12.

In some embodiments, the heavy chain variable region comprises an amino acid residue substitution at at least one (e.g., one, two, or three) of R16, S98, and V108, wherein the positions are numbered with respect to SEQ ID NO: 11. In some embodiments, the light chain variable region comprises an amino acid residue substitution at at least one (e.g., one, two, three, or four) of positions S82, N91, L93, and 196, wherein the positions are numbered with respect to SEQ ID NO: 12. In some embodiments, the heavy chain variable region comprises amino acid substitutions at any of the residue positions or combinations of residue positions represented in Table 12, below. In some embodiments, the light chain variable region comprises amino acid substitutions at any of the residue positions or combinations of residue positions represented in Table 12, below. In some embodiments, both the heavy chain variable region comprises any of the the heavy chain variable region amino acid substitutions or combinations of amino acid substitutions at any of the residue positions or combinations of residue positions represented in Table 12, below, and the light chain variable region comprises any of the amino acid substitutions or combinations of the light chain variable region amino acid substitutions at any of the residue positions or combinations of residue positions represented in Table 12, below.

In some embodiments, the heavy chain variable region comprises at least one of the amino acid residue substitutions R16G, S98R, V108D, or any combination thereof, wherein the positions are numbered with respect to SEQ ID NO: 11. In some embodiments, the heavy chain variable region comprises two or three of the above substitutions. In some embodiments, the light chain variable region comprises at least one of the amino acid residue S82A, N91C, N91S, L93Y, I96S, or any combination thereof, wherein the positions are numbered with respect to SEQ ID NO:12. In some embodiments, the light chain variable region comprises two, three, four, or five of the above substitutions. In some embodiments of the antibody described in this section, both the light and the heavy chains comprise substitutions. In some embodiments, the heavy chain variable region comprises any of the amino acid substitutions or combinations of amino acid substitutions represented in Table 12, below. In some embodiments, the light chain variable region comprises any of the amino acid substitutions or combinations of amino acid substitutions represented in Table 12, below. In some embodiments, both the heavy chain variable region comprises any of the the heavy chain variable region amino acid substitutions or combinations of amino acid substitutions represented in Table 12, below, and the light chain variable region comprises any of the amino acid substitutions or combinations of the light chain variable region amino acid substitutions represented in Table 12, below.

7. Antibodies and Antigen-Binding Portions Thereof Derived from the C143 Patient-Derived Antibody.

In some embodiments, the antibodies and antigen-binding portions thereof that specifically bind to coronavirus spike protein (e.g., a SARS-CoV-2 spike protein) are derived from the C143 patient-derived antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:32, a CDRH2 comprising at least 70% identity to SEQ ID NO:33, and a CDRH3 comprising at least 70% identity to SEQ ID NO:34 and/or a light chain variable region comprising a CDRL1 comprising at least 70% identity to SEQ ID NO:52, a CDRL2 comprising at least 70% identity to SEQ ID NO:53, and a CDRL3 comprising at least 70% identity to SEQ ID NO:54. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 13 and/or a light chain variable region comprising at least 70% identity to SEQ ID NO: 14.

In some embodiments, the heavy chain variable region comprises an amino acid residue substitution at at least one (e.g., one, two, three, four, or more) of V29, K32, L51, D57, A77, and G91, wherein the positions are numbered with respect to SEQ ID NO:13. In some embodiments, the light chain variable region comprises an amino acid residue substitution at at least one (e.g., one, two, three, four, or more) of positions N27, T33, L34, Y41, G53, S57, G82, and A96, wherein the positions are numbered with respect to SEQ ID NO: 14. In some embodiments, the heavy chain variable region comprises amino acid substitutions at any of the residue positions or combinations of residue positions represented in Table 13, below. In some embodiments, the light chain variable region comprises amino acid substitutions at any of the residue positions or combinations of residue positions represented in Table 13, below. In some embodiments, both the heavy chain variable region comprises any of the the heavy chain variable region amino acid substitutions or combinations of amino acid substitutions at any of the residue positions or combinations of residue positions represented in Table 13, below, and the light chain variable region comprises any of the amino acid substitutions or combinations of the light chain variable region amino acid substitutions at any of the residue positions or combinations of residue positions represented in Table 13, below.

In some embodiments, the heavy chain variable region comprises at least one of the amino acid residue substitutions V29F, K32Y, L51Y, D57T, A77T, G91A, or any combination thereof, wherein the positions are numbered with respect to SEQ ID NO:13. In some embodiments, the heavy chain variable region comprises at least two, three, four, five, or six of the above substitutions. In some embodiments, the light chain variable region comprises at least one of the amino acid residue N27S, T33N, L34Y, Y41H, G53V, S57P, G82A, A96S, or any combination thereof, wherein the positions are numbered with respect to SEQ ID NO: 14. In some embodiments, the light chain variable region comprises at least two, three, four, five, six, seven, eight of the above substitutions. In some embodiments of the antibody described in this section, both the light and the heavy chains comprise substitutions. In some embodiments, the heavy chain variable region comprises any of the amino acid substitutions or combinations of amino acid substitutions represented in Table 13, below. In some embodiments, the light chain variable region comprises any of the amino acid substitutions or combinations of amino acid substitutions represented in Table 13, below. In some embodiments, both the heavy chain variable region comprises any of the the heavy chain variable region amino acid substitutions or combinations of amino acid substitutions represented in Table 13, below, and the light chain variable region comprises any of the amino acid substitutions or combinations of the light chain variable region amino acid substitutions represented in Table 13, below.

8. Antibodies and Antigen-Binding Portions Thereof Derived from the LY-1404 Antibody.

In some embodiments, the antibodies and antigen-binding portions thereof that specifically bind to coronavirus spike protein (e.g., a SARS-CoV-2 spike protein) are derived from LY-1404 antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:62, a CDRH2 comprising at least 70% identity to SEQ ID NO:63, and a CDRH3 comprising at least 70% identity to SEQ ID NO:64 and/or a light chain variable region comprising a CDRL1 comprising at least 70% identity to SEQ ID NO:68, a CDRL2 comprising at least 70% identity to SEQ ID NO:69, and a CDRL3 comprising at least 70% identity to SEQ ID NO:70. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:58 and/or a light chain variable region comprising at least 70% identity to SEQ ID NO:59.

In some embodiments, the heavy chain variable region comprises an amino acid residue substitution at at least one (e.g., one, two, three, four, or more) of D88, V90, S62, V81, F24, 131, H99, T79, or 1105, wherein the positions are numbered with respect to SEQ ID NO:58. In some embodiments, the light chain variable region comprises an amino acid residue substitution at at least one (e.g., one, two, three, four, or more) of positions A98, Q39, T5, K47, F51, K44, M49, or Q6, wherein the positions are numbered with respect to SEQ ID NO:59. In some embodiments, the heavy chain variable region comprises amino acid substitutions at any of the residue positions or combinations of residue positions represented in Table 14, below. In some embodiments, the light chain variable region comprises amino acid substitutions at any of the residue positions or combinations of residue positions represented in Table 14, below. In some embodiments, both the heavy chain variable region comprises any of the the heavy chain variable region amino acid substitutions or combinations of amino acid substitutions at any of the residue positions or combinations of residue positions represented in Table 14, below, and the light chain variable region comprises any of the amino acid substitutions or combinations of the light chain variable region amino acid substitutions at any of the residue positions or combinations of residue positions represented in Table 14, below.

In some embodiments, the heavy chain variable region comprises at least one of amino acid residue substitutions D89Q, V90S, S62N, V81T, F24Y, 131T, H98Y, T70S, I105L, or any combination thereof, wherein the positions are numbered with respect to SEQ ID NO:58. In some embodiments, the heavy chain variable region comprises two, three, four, five, six, seven, eight, or nine of the above substitutions. In some examples, the heavy chain variable region comprises substitutions D88Q, V90S, S62N, V81T, F24Y, 131T, H99Y, and T70S. In some examples, the heavy chain variable region comprises substitutions D88Q, V90S, S62N, V81T, F24Y, 131T, and H99Y. In some examples, the heavy chain variable region comprises substitutions D88Q, V90S, S62N, V81T, F24Y, H99Y, and T70S. In some examples, the heavy chain variable region comprises substitutions D88Q, V90S, S62N, V81T, F24Y, 131T, and T70S. In some examples, the heavy chain variable region comprises substitutions D88Q, V90S, S62N, V81T, 131T, H99Y, and T70S. In some embodiments, the light chain variable region comprises at least one of amino acid residue substitutions A98I, Q39K, T5Q, K47E, F51Y, K44E, M49L, Q6S, or any combination thereof, wherein the positions are numbered with respect to SEQ ID NO:59. In some embodiments, the light chain variable region comprises two, three, four, five, six, seven, or eight of the above substitutions. In some embodiments of the antibody described in this section, both the light and the heavy chains comprise substitutions. In some examples, the antibody comprises the substitutions V90S and E85A. In some examples, the antibody comprises the substitutions S62N and E85A. In some examples, the antibody comprises the substitutions T70S and E85A. In some examples, the antibody comprises the substitutions V90S and Q39K. In some examples, the antibody comprises the substitutions S62N and Q39K. In some examples, the antibody comprises the substitutions T70S and Q39K. In some embodiments, the heavy chain variable region comprises any of the amino acid substitutions or combinations of amino acid substitutions represented in Table 14, below. In some embodiments, the light chain variable region comprises any of the amino acid substitutions or combinations of amino acid substitutions represented in Table 14, below. In some embodiments, both the heavy chain variable region comprises any of the the heavy chain variable region amino acid substitutions or combinations of amino acid substitutions represented in Table 14, below, and the light chain variable region comprises any of the amino acid substitutions or combinations of the light chain variable region amino acid substitutions represented in Table 14, below.

9. Antibodies and Antigen-Binding Portions Thereof Derived from the SA58 Antibody.

In some embodiments, the antibodies and antigen-binding portions thereof that specifically bind to coronavirus spike protein (e.g., a SARS-CoV-2 spike protein) are derived from SA58 antibody, as described in Example 2 herein. In some embodiments, the antibodies or antigen-binding portions thereof comprise a heavy chain variable region comprising a CDRH1 comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:65, a CDRH2 comprising at least 70% identity to SEQ ID NO:66, and a CDRH3 comprising at least 70% identity to SEQ ID NO:67 and/or a light chain variable region comprising a CDRL1 comprising at least 70% identity to SEQ ID NO:71, a CDRL2 comprising at least 70% identity to SEQ ID NO:72, and a CDRL3 comprising at least 70% identity to SEQ ID NO:73. In some embodiments, the antibodies or antigen-binding portions thereof comprise a heavy chain variable region comprising at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:60 and/or a light chain variable region comprising at least 70% identity to SEQ ID NO:61.

In some embodiments, the heavy chain variable region comprises an amino acid residue substitution at at least one (e.g., one, two, three, four, or more) of T53, A61, or E10Q, wherein the positions are numbered with respect to SEQ ID NO:60. In some embodiments, the light chain variable region comprises an amino acid residue substitution at at least one (e.g., one, two, three, four, or more) of positions N95, S85, S54, or M4, wherein the positions are numbered with respect to SEQ ID NO:61. In some embodiments, the heavy chain variable region comprises amino acid substitutions at any of the residue positions or combinations of residue positions represented in Table 15, below. In some embodiments, the light chain variable region comprises amino acid substitutions at any of the residue positions or combinations of residue positions represented in Table 15, below. In some embodiments, both the heavy chain variable region comprises any of the the heavy chain variable region amino acid substitutions or combinations of amino acid substitutions at any of the residue positions or combinations of residue positions represented in Table 15, below, and the light chain variable region comprises any of the amino acid substitutions or combinations of the light chain variable region amino acid substitutions at any of the residue positions or combinations of residue positions represented in Table 15, below.

In some embodiments, the heavy chain variable region comprises at least one of the amino acid residue substitutions T53L, A61S, E10Q, or any combination thereof, wherein the positions are numbered with respect to SEQ ID NO:60. In some embodiments, the heavy chain variable region comprises two or three of the above substitutions. In some embodiments, the light chain variable region comprises at least one of the amino acid residue substitutions N95V, S85A, S54T, M4V, or any combination thereof, wherein the positions are numbered with respect to SEQ ID NO:61. In some embodiments, the light chain variable region comprises two, three, or four of the above substitutions. In some embodiments of the antibody described in this section, both the light and the heavy chains comprise substitutions. In some examples, the antibody comprises the substitutions T53L and N95V. In some examples, the antibody comprises the substitutions Q82T and N95V. A61S and N95V. In some examples, the antibody comprises the substitutions T53L and M4V. In some examples, the antibody comprises the substitutions Q82T and M4V. A61S and M4V. In some examples, the antibody comprises the substitutions T53L and A61S. In some embodiments, the heavy chain variable region comprises any of the amino acid substitutions or combinations of amino acid substitutions represented in Table 15, below. In some embodiments, the light chain variable region comprises any of the amino acid substitutions or combinations of amino acid substitutions represented in Table 15, below. In some embodiments, both the heavy chain variable region comprises any of the the heavy chain variable region amino acid substitutions or combinations of amino acid substitutions represented in Table 15, below, and the light chain variable region comprises any of the amino acid substitutions or combinations of the light chain variable region amino acid substitutions represented in Table 15, below.

### III. Antibody Expression and Purification, Nucleic Acids, Vectors, and Cells

The viral antigen-specific antibodies and antigen binding fragments thereof discussed above may be produced by recombinant expression in a human or non-human cell. Synthetic antibody-producing cells include non-human cells expressing heavy chains, light chains, or both heavy and light chains; human cells that are not immune cells expressing heavy chains, light chains, or both heavy and light chains; and human B cells that produce heavy chains or light chains, but not both heavy and light chains. Synthetic antibodies of this disclosure may be heterologously expressed, in vitro or in vivo, in cells other than human B cells, such as non-human cells and human cells other than B cells, optionally other than immune cells, and optionally in cells other than cells in a B cell lineage.

The viral antigen-specific antibodies and antigen binding fragments thereof and molecules comprising them described herein can be produced using a variety of techniques known in the art of molecular biology and protein chemistry. For example, a nucleic acid encoding the antibody or antigen binding fragment thereof can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments thereof include yeast, bacteria, insect, plant, and mammalian cells. In some embodiments, the antibodies or antigen binding fragments thereof are expressed from cells from bacteria such as E. coli, fungi such as Saccharomyces cerevisiae and Pichia pastoris, insect cells such as SF9, mammalian cell lines (e.g., human cell lines), or primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Also provided herein are nucleic acid molecules encoding a viral antigen-specific antibody or antigen binding portion thereof that binds specifically to a viral antigen as described in this disclosure.

In some embodiments, provided are nucleic acid molecules encoding antibodies or antigen binding fragments thereof that bind specifically to a viral antigen, wherein the nucleic acid sequences comprise sequences encoding an amino acid sequence that is at least 70% identical (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical) to any of the sequences in Table 1, Table 2, and/or Table 3 and comprise an amino acid substitution as recited in the embodiments of antibodies and antigen binding portions thereof as set forth in the Summary of this disclosure.

In some embodiments, the nucleic acid molecules encoding the viral antigen-specific antibodies or antigen binding fragments thereof are synthetic sequences designed for expression in a host cell (for example, a human cell).

In some embodiments, the nucleic acid molecules encoding the viral antigen-specific antibodies or antigen binding fragments thereof are operably linked to a promoter capable of directing expression in a bacterial cell or a eukaryotic cell.

Also provided herein are DNA constructs comprising a promoter that drives expression in a host cell operably linked to a recombinant nucleic acid molecule comprising a nucleotide sequence that encodes a viral antigen-specific specific antibody or antigen binding fragment thereof.

Also provided herein are vectors, discussed further below, comprising a DNA construct comprising a promoter that drives expression in a host cell operably linked to a recombinant nucleic acid molecule comprising a nucleotide sequence that encodes a viral antigen-specific specific antibody or antigen binding fragment thereof.

Also provided herein are host cells, including bacterial host cells and eukaryotic host cells, comprising a recombinant nucleic acid molecule encoding a viral antigen-specific antibody or antigen binding fragment thereof as described in this disclosure.

In vitro methods are also suitable for preparing monovalent antibodies or antigen binding fragments thereof. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or fragments is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### IV. Pharmaceutical Compositions and Formulations

The viral antigen-specific antibodies and antigen binding portions thereof described herein are suitable for administration in vitro or in vivo. Compositions comprising a viral antigen-specific antibody or antigen binding fragment thereof of the present disclosure and a pharmaceutically acceptable carrier (excipient) are provided. A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The compositions may further comprise a diluent, solubilizer, emulsifier, preservative, and/or adjuvant to be used with the methods disclosed herein. Such compositions can be used, for example, in a subject with a viral infection that would benefit from any of the viral antigen-specific antibodies or antigen binding fragments thereof described herein.

Suitable carriers and their formulations are described in Remington: The Science and Practice of Pharmacy, 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically-acceptable salt to render the formulation isotonic. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the viral antigen-specific antibody or antigen binding fragment thereof.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a viral antigen-specific antibody or antigen binding fragment thereof disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising a viral antigen-specific antibody or antigen binding fragment thereof disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions or suspensions, including saline and buffered media) comprising a viral antigen-specific antibody or antigen binding fragment thereof in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, the compositions can be selected for inhalation or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5.

In certain embodiments, a pharmaceutical composition can comprise an effective amount of a viral antigen-specific antibody or antigen binding fragment thereof in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a viral antigen-specific antibody or antigen binding fragment thereof in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E. et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15: 167-277; and Langer (1982) Chem Tech 12:98-105), ethylene vinyl acetate (Hsu and Langer (1985) J Biomed Materials Res 19(4):445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In still another aspect, unit dose forms comprising a viral antigen-specific antibody or antigen binding fragment thereof as described in this disclosure are provided. A unit dose form can be formulated for administration according to any of the routes described in this disclosure. In one example, the the unit dose form is formulated for intravenous or intraperitoneal administration. In still another aspect, pharmaceutical packages comprising unit dose forms of a viral antigen-specific antibody or antigen binding fragment thereof are provided.

### V. Kits and Packaging

The viral antigen-specific antibodies and antigen binding fragments thereof disclosed herein are ideally suited for the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In some instances, one of the containers may comprise a viral antigen-specific antibody or antigen binding fragment thereof as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. For example, a kit for detecting a viral antigen in a subject with a viral infection is provided herein. In some embodiments, the kit comprises a container containing a labeled viral antigen-specific antibody or antigen binding fragment thereof. In some embodiments, the kit comprises separate containers containing a viral antigen-specific antibody or antigen binding fragment thereof and a detectable label.

A viral antigen-specific antibody or antigen binding fragment thereof as described in this disclosure for use in treating subjects with viral infections may be delivered in a pharmaceutical package or kit to doctors and cancer patients. Such packaging is intended to improve patient convenience and compliance with the treatment plan. Typically the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a viral antigen-specific antibody or antigen binding fragment. In some embodiments, the pharmaceutical package or kit further comprises unit dose forms of one or more additional viral therapeutics.

In one embodiment, the kit or pharmaceutical package comprises a viral antigen-specific antibody or antigen binding fragment in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### VI. Methods of Use

Provided herein are methods to treat, inhibit, or ameliorate a viral infection in a subject using a viral antigen-specific antibody or antigen binding fragment thereof as described in this disclosure. The methods comprise administering to a subject a pharmaceutically effective amount of a composition comprising an isolated viral antigen-specific antibody or antigen binding portion thereof described herein. Also, provided are prognostic and diagnostic methods for viral infections based on detection and/or quantitation of a viral antigen using a viral antigen-specific antibody or antigen binding fragment as described in this disclosure. Also provided are methods of detecting the presence of a virus in a sample using the described viral antigen-specific antibodies or antigen binding fragments.

The compositions described herein are useful in, inter alia, methods for treating a viral infection (e.g., a coronavirus infection, an ebolavirus infection, and/or an influenza A infection) in a subject. As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and the term patient or subject includes human and veterinary subjects. The viral antigen-specific antibody or antigen binding portion thereof described herein are useful for treating viral infections in humans, including, without limitation, pediatric and geriatric populations, and in animals, e.g., veterinary applications. In one embodiment, the subject is a human.

### A. Methods of Treatment

Provided herein are methods to treat a viral infection in a subject using a viral antigen-specific antibody or antigen binding fragment thereof as described in this disclosure. In some instances, the viral antigen-specific antibody or antigen binding fragment thereof may directly inhibit viral entry into host cells. In some instances, the viral antigen-specific antibody or antigen binding fragment thereof may inhibit viral replication.

In some embodiments, the subject has or is suspected to have a viral infection. In some embodiments, the subject is diagnosed with a viral infection. In some embodiments, the subject is a human that is suspected of having a viral infection. In some embodiments, the subject has or is suspected to have a viral infection. In some embodiments, the subject has symptoms indicative of a viral infection. In some embodiments, the subject is diagnosed as having a viral infection.

In some embodiments, the subject may be asymptomatic or symptomatic. The subject may be male or female and may be a juvenile or an adult (e.g., at least 30 years old, at least 40 years old, or at least 50 years old). In some embodiments, the subject is displaying one or more symptoms indicative of a coronavirus infection (e.g., a SARS-CoV-2 or SARS-CoV-2 variant infection. Such symptoms include, but are not limited to, any of a new loss of taste or smell, myalgia, fatigue, shortness of breath or difficulty breathing, fever, and/or cough. Symptoms may also include pharyngitis, headache, productive cough (i.e. a cough that produces mucus or phlegm), gastrointestinal symptoms (e.g., diarrhea, nausea, vomiting, or abdominal pain), hemoptysis, chest pressure or pain, confusion, cyanosis, and/or chills. In some embodiments, the patient has at least two symptoms selected from the group consisting of a new loss of taste or smell, shortness of breath or difficulty breathing, fever, cough, chills, or muscle aches. In some embodiments, the patient may have a blood oxygen level reading of 94 or less, e.g., as determined by an oximeter. In some embodiments, the subject may have radiographic evidence of pulmonary infiltrates. In some embodiments, the subject may have been receiving standard support care, e.g., such as being administered oxygen, fluids, and/or other therapeutic procedures or agents.

In some embodiments, the subject is displaying one or more symptoms indicative of an ebolavirus infection. Such symptoms include, but are not limited to, any of fever, aches and pains (e.g., severe headache, muscle pain, joint pain), weakness and fatigue, sore throat, loss of appetite, gastrointestinal symptoms (e.g., abdominal pain, diarrhea, vomiting), unexplained hemorrhaging, unexplained bleeding, unexplained bruising, red eyes, skin rash, and/or hiccups.

In some embodiments, the subject is displaying one or more symptoms indicative of an influenza A infection. Such symptoms include, but are not limited to, any of fever or feeling feverish, chills, cough, sore throat, runny or stuffy nose, muscle or body aches, headaches, fatigue, vomiting, and/or diarrhea.

In some embodiments, the subject may not manifest any symptoms that are typically associated with a viral infection. In some cases the subject is known or believed to have been exposed to a virus (e.g., a coronavirus, an ebolavirus, and/or an influenza A virus), suspected of having exposure to a virus or believed not to have had exposure to a virus. In some cases, the subject may have recovered from a prior viral infection. In some cases, the subject has received a viral vaccine. In some cases, the subject has been free of symptoms suggestive of a viral infection for at least 14 days. In some cases, the subject may have one or more of other conditions of hypertension, coronary artery disease, diabetes, chronic obstructive pulmonary disease.

A coronavirus infection (e.g., a SARS-CoV-2 infection) in a subject can be detected by various assays performed on a biological sample from the subject. The biological sample may be from a throat swab, a nasopharyngeal swab, sputum or tracheal aspirate, urine, feces, or blood. In some instances, nucleic acids are isolated from the biological sample and tested for the presences of viral genomic sequences. In some embodiments, PCR is performed to detect coronavirus nucleic acids from the biological sample. In some embodiments, a subject may have antibodies that selectively bind to coronavirus proteins, e.g., coronavirus spike protein. Antibodies can be detected in a blood sample from the subject by immunoassay (e.g., lateral flow assay or ELISA). In some embodiments, coronavirus infection can be detected using a proximity-based binding assay for detection of virus and/or anti-virus antibodies, as described in Lui, I., et al., “Trimeric SARS-CoV-2 Spike interacts with dimeric ACE2 with limited intra-Spike avidity,” bioRxiv, doi.org/10.1101/2020.05.21.109157, published May 21, 2020 and Elledge et al., 2021, “Engineering luminescent biosensors for point-of-care SARS-CoV-2 antibody detection,” Nat. Biotech., doi:10.1038/s41587-021-00878-8.

An ebolavirus infection in a subject can be detected by various assays performed on a biological sample from the subject. The biological sample may be from a throat swab, a nasopharyngeal swab, sputum or tracheal aspirate, urine, feces, or blood. In some instances, nucleic acids are isolated from the biological sample and tested for the presences of viral genomic sequences. In some embodiments, PCR is performed to detect ebolavirus nucleic acids from the biological sample. In some embodiments, a subject may have antibodies that selectively bind to ebolavirus proteins, e.g., ebolavirus glycoprotein. Antibodies can be detected in a blood sample from the subject by immunoassay (e.g., lateral flow assay or ELISA).

An influenza A infection in a subject can be detected by various assays performed on a biological sample from the subject. The biological sample may be from a throat swab, a nasopharyngeal swab, sputum or tracheal aspirate, urine, feces, or blood. In some instances, nucleic acids are isolated from the biological sample and tested for the presences of viral genomic sequences. In some embodiments, PCR is performed to detect influenza A nucleic acids from the biological sample. In some embodiments, a subject may have antibodies that selectively bind to influenza A proteins, e.g., HA. Antibodies can be detected in a blood sample from the subject by immunoassay (e.g., lateral flow assay or ELISA). In some embodiments, rapid influenza diagnostic tests are used to detect viral antigens. In some embodiments, immunofluorescence assays are used to detect influenza A infection.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., a viral infection, such as a coronavirus infection, an ebolavirus infection, and/or an influenza A infection, lessening in the severity or progression, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying, preventing increases in, or decreasing viral load. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a viral infection in a subject by administering a viral antigen-specific antibody or antigen binding fragment thereof as described in this disclosure is considered to be a treatment if there is a 10% reduction in one or more symptoms of the viral infection in a subject as compared to a control. Thus the reduction can be a 10%, 20%, 30%, 40%, 50%, 60%, 70%, 80%, 90%, 100%, or any percent reduction in between 10% and 100% as compared to native or control levels. In some embodiments, formulations comprising a viral antigen-specific antibody or antigen binding fragment thereof as described herein are administered to the subject until the subject exhibits amelioration of at least one symptom of a viral infection and/or is demonstrated to have a sustained decrease in viral load, e.g., as measured by immunoassay and/or quantitative amplification method, including PCR or sequencing. In some instances, the formulation is administered to the subject until viral load is undetectable, i.e. below the level of detection, such that no viral nucleic acid copies can be detected by the assay methodology employed. In some instances, the subject exhibits undetectable viral load 1-4 weeks, 2-4 weeks, 2-12 weeks, 4-12 weeks, or 12-24 weeks after last administration of the formulation. It is understood that treatment does not necessarily refer to a cure or complete ablation of the disease, condition, or symptoms of the disease or condition.

In some embodiments, the subject is administered a viral antigen-specific antibody or antigen binding fragment thereof as described herein within 1, 2, 3, 4, or 5 days from the onset of symptoms or within 1, 2, 3, 4, or 5 days from testing positive for a viral infection (e.g., a coronavirus infection, an ebolavirus infection, and/or an influenza A infection). In some embodiments, the subject is administered a viral antigen-specific antibody or antigen binding fragment thereof as described herein within 1 or 2 days of hospitalization with one or more symptoms indicative of a viral infection.

In some embodiments, the viral antigen-specific antibody or antigen binding fragment thereof is administered to the subject at least once a day, at least twice a day, or at least three times a day. In some embodiments, the viral antigen-specific antibody or antigen binding fragment thereof is administered on consecutive days or on non-consecutive days. In some instances, the viral antigen-specific antibody or antigen binding fragment thereof is administered to the subject for at least 1 day, at least 2 days, at least 4 days, at least 5 days, at least 6 days, at least 1 week, at least 2 weeks, at least 3 weeks, at least 1 month, at least 2 months, or at least 3 months. In some embodiments, the viral antigen-specific antibody or antigen binding fragment thereof is administered to the subject for 2 to 5 or more days after the viral load is undetectable in order avoid “rebound” of virus replication.

A pharmaceutical preparation as described herein can comprise an effective amount of a viral antigen-specific antibody or antigen binding fragment thereof described herein. Such effective amounts can be readily determined by one of ordinary skill in the art as described below. Considerations include the effect of the administered viral antigen-specific antibody or antigen binding fragment thereof, or the combinatorial effect of the viral antigen-specific antibody or antigen binding fragment thereof with one or more additional active agents, if more than one agent is used in or with the pharmaceutical composition.

The term “administer,” as used herein, refers to a method of delivering agents, compounds, or compositions to the desired site of biological action. The pharmaceutical compositions (e.g., as described above) are prepared for administration in a number of ways, including but not limited to injection, ingestion, transfusion, implantation, or transplantation, depending on whether local or systemic treatment is desired, and on the area to be treated. The preparation of such pharmaceutically acceptable compositions is within the ability of one skilled in the art. The compositions are administered via any of several routes of administration, including topical, oral, parenteral, intravenous, intra-articular, intraperitoneal, intramuscular, subcutaneous, intracavity, intralesional, transdermal, intradermal, intrahepatical, intrathecal, intracranial, rectal, transmucosal, intestinal, ocular, otic, nasal, inhalation, or intrabronchial delivery, or any other method known in the art. In some embodiments, the viral antigen-specific antibody or antigen binding fragment thereof is administered orally, intravenously, or intraperitoneally. When a viral infection, or a symptom thereof, is being treated, administration of the substance typically occurs after the onset of the viral infection or symptoms thereof. When a viral infection, or symptoms thereof, are being prevented, delayed, or reduced in severity, administration of the substance typically occurs before the onset of the viral infection or symptoms thereof. Administration encompasses direct administration, such as administration to a subject by a medical professional or self-administration, or indirect administration, which may be the act of prescribing a composition described in the present disclosure.

In one aspect, provided is a method of treating a subject with a viral infection, the method comprising administering to the patient a pharmaceutically effective amount of a composition comprising a viral antigen-specific antibody or antigen binding fragment thereof as described in this disclosure. The composition may further comprise a pharmaceutically acceptable carrier.

In some instances, the patient is administered an isolated viral antigen-specific antibody or antigen binding fragment thereof. The term “isolated,” as used with reference to a protein (or nucleic acid), denotes that the protein (or nucleic acid) is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated protein (or nucleic acid) is at least 85% pure, at least 90% pure, at least 95% pure, or at least 99% pure.

In some instances, the viral antigen-specific antibody or antigen binding fragment thereof can be administered via virus-like particles. Virus-like particles (VLPs) comprise viral protein(s) derived from the structural proteins of a virus. Methods for making and using virus like particles are described in, for example, Garcea and Gissmann, Current Opinion in Biotechnology 15:513-7 (2004).

In some instances, the viral antigen-specific antibody or antigen binding fragment thereof can be administered by subviral dense bodies (DBs). DBs transport proteins into target cells by membrane fusion. Methods for making and using DBs are described in, for example, Pepperl-Klindworth et al., Gene Therapy 10:278-84 (2003).

In some instances, the viral antigen-specific antibody or antigen binding fragment thereof can be administered by tegument aggregates. Methods for making and using tegument aggregates are described in International Publication No. WO 2006/110728.

In another aspect, provided is a method of treating a subject with a viral infection, the method comprising administering to the patient cells that have been genetically engineered, using methods such as those described herein, to express and secrete a viral antigen-specific antibody or antigen binding fragment thereof as described in this disclosure.

In another aspect, provided is a method of treating a subject with a viral infection, the method comprising administering to the patient a vector comprising a nucleic acid sequence encoding the viral antigen-specific antibody or antigen binding fragment thereof as described in this disclosure.

There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein.

As used herein, plasmid or viral vectors are agents that transport the disclosed nucleic acids into the cell without undesired degradation and include a promoter yielding expression of the nucleic acid molecule and/or adapter polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors. In some instances, the nucleic acid molecules encoding the viral antigen-specific antibody or antigen binding fragment thereof can be delivered via virus-like particles.

Non-viral based delivery methods, can include expression vectors comprising nucleic acid molecules and nucleic acid sequences encoding the adapter polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, WI), Clonetech (Pal Alto, CA), Stratagene (La Jolla, CA), and Invitrogen/Life Technologies (Carlsbad, CA). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters (e.g., β-actin promoter or EF1α promoter), or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the beta-actin promoter, the EF1A promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, CT) sequences typically are expressed as a fusion with the encoded polypeptide. Such tags can be inserted anywhere within the polypeptide including at either the carboxyl or amino terminus.

As used herein, a “therapeutically effective amount” means the amount of an agent that is effective for producing a desired effect in a subject, e.g., to reduce viral load or prevent viral load from increasing, to reduce or ameliorate at least one symptom of a viral infection (e.g., a coronavirus infection, an ebolavirus infection, or an influenza A infection), and/or otherwise reduce the length of time that a patient experiences a symptom of a viral infection, or extend the length of time before a symptom may recur. The actual dose that comprises the effective amount may depend upon the route of administration, the size and health of the subject, the viral infection being treated (e.g., a SARS-CoV-2 infection), and the like.

In certain embodiments, the effective amount of a pharmaceutical composition comprising a viral antigen-specific antibody or antigen binding fragment thereof to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a viral antigen-specific antibody or antigen binding fragment thereof is being used, the route of administration, and the size (body weight, body surface or organ size) and/or condition (the age and general health) of the patient. The clinician can titer the dosage and modify the route of administration to obtain the optimal therapeutic effect. An effective amount is also one in which any toxic or detrimental effects of the composition are outweighed by the therapeutically beneficial effects. In some instances, an effective amount is not a dosage so large as to cause adverse side effects, such as hyperviscosity syndromes, pulmonary edema, congestive heart failure, and the like. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. It should also be understood that a specific dosage and treatment regimen for any particular subject also depends upon the judgment of the treating medical practitioner (e.g., doctor or nurse). The dosage of the effective amount may be adjusted by the individual physician or veterinarian in the event of any complication.

The clinician also selects the frequency of dosing, taking into account the pharmacokinetic parameters of the viral antigen-specific antibody or antigen binding fragment thereof in the formulation used. Such pharmacokinetic parameters are well known in the art, i.e., the rate of absorption, bioavailability, metabolism, clearance, and the like (see, e.g., Hidalgo-Aragones (1996) J Steroid Biochem. Mol. Biol. 58:611-617; Groning (1996) Pharmazie 51:337-341; Fotherby (1996) Contraception 54:59-69; Johnson (1995) J Pharm. Sci. 84:1144-1146; Rohatagi (1995) Pharmazie 50:610-613; Brophy (1983) Eur. J Cin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In some instances, a therapeutically effective amount may vary from about 0.0001 to 100 mg/kg, and more usually 0.01 to 20 mg/kg, of the patient's body weight. For example dosages can be 0.3 mg/kg body weight, 1 mg/kg body weight, 3 mg/kg body weight, 5 mg/kg body weight, 10 mg/kg body weight or within the range of 0.1-20 mg/kg. In certain examples, the viral antigen-specific antibody or antigen binding fragment thereof can be administered at a dose of 1 mg/kg, 2 mg/kg, 3 mg/kg, 4 mg/kg, or 5 mg/kg in one or more dose administrations daily, for one or several days.

Toxicity and therapeutic efficacy of the viral antigen-specific antibodies and antigen binding portions thereof described herein can be determined by known pharmaceutical procedures in cell cultures or experimental animals (e.g., animal models of any of the disease states described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. An antibody or antigen binding fragment thereof that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of an antibody or antigen binding portion thereof lies generally within a range of circulating concentrations of the antibody or antigen binding portion thereof that includes the ED₅₀ with little or no toxicity. The dosage may vary within this range depending upon the dosage form employed and the route of administration utilized. For antibody or antigen binding portion thereof described herein, the therapeutically effective dose can be estimated initially from cell culture assays. A dose can be formulated in animal models to achieve a circulating plasma concentration range that includes the EC₅₀ (i.e., the concentration of the construct—e.g., polypeptide—that achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

Suitable human doses of any of the antibody or antigen binding portion thereof described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

In certain embodiments, the route of administration of the pharmaceutical composition is in accord with known methods, e.g. orally, through injection by intravenous, intraperitoneal, intracerebral (intra-parenchymal), intracerebral, intraventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of a combination therapy may be administered by different routes.

In certain embodiments, the composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In some instances, the provided methods may include administering to the subject a viral antigen-specific antibody or antigen binding fragment thereof that is conjugated to a therapeutic agent (e.g., an anti-viral therapeutic agent). In some instances, the viral antigen-specific antibody or antigen binding fragment thereof can be labeled, conjugated, or fused with a therapeutic agent or diagnostic agent (such as an imaging agent). The linkage can be covalent or noncovalent (e.g., ionic). Such antibodies and antibody fragments are referred to antibody-drug conjugates (ADC) or immunoconjugates.

In some instances, the provided methods may include administering a viral antigen-specific antibody or antigen binding fragment thereof and a second form of anti-viral therapy to the subject.

### B. Diagnostic Methods

In another aspect, provided are methods for detecting the presence of a virus in a biological sample comprising: (a) contacting said sample with a composition comprising an isolated viral antigen-specific antibody or antigen binding portion thereof as described in this disclosure; and (b) detecting an amount of binding of the isolated antibody or antigen binding portion thereof as a determination of the presence of said virus. In some embodiments, the biological sample comprises a throat swab, a nasopharyngeal swab, sputum or tracheal aspirate, urine, feces, or blood. In some embodiments, the composition further comprises a pharmaceutically acceptable carrier.

Also provided are methods to diagnose viral infections in a subject. Specifically, the diagnosis may be of a coronavirus infection (e.g., a SARS-CoV-2 infection), an ebolavirus infection, and/or an influenza A infection. The method may comprise assaying a sample from a subject for the presence of viral antigen and diagnosing the subject with a viral infection if the viral antigen is detected in the sample. In some instances, the method comprises assaying a biological sample from a subject for the presence of viral antigen using a viral antigen-specific antibody or antigen binding fragment thereof and determining if the subject has a viral infection. Conversely, if the amount of viral antigen in the sample is low (i.e., below background levels) or undetectable, the subject may not be diagnosed with a viral infection.

### VII. Machine Learning to Predict Likely Variants

Some embodiments can use machine learning to predict antibody variants that are likely to occur in nature. Such variants can be used (selected) to improve properties of the antibodies. Such embodiments can use directed evolution guided by a language model. Example properties that may be improved include binding affinity, higher neutralization, thermostability preserved or increased, and solubility.

Protein language models can be implemented with neural network algorithms that are trained on up to millions of protein sequences to learn which amino acids are more likely to appear together to form a valid protein. Recent work has demonstrated that language models can predict natural evolution despite having no knowledge of protein-specific selection pressures [8]; however, this prior work only predicted the direction of evolution retrospectively when given full knowledge of the evolutionary trajectory. The present disclosure shows that language models can predict unobserved evolution to prospectively design (predict) new proteins.

### A. Individual Language Models

A language according to the present disclosure can receive the wildtype sequence (or other antibody sequence being analyzed) as input. Multiple language models can be used, with each receiving an input of the wildtype sequence. As examples, we used the ESM-1b and ESM-1v language models, obtained from github.com/facebookresearch/esm. These language models were trained on UniRef50 (ESM-1b) or UniRef90 (ESM-1v): www.uniprot.org/help/uniref. These language models output the likelihood of seeing an amino acid at a residue given the rest of the sequence as input context.

Various training techniques may be used to train the language models. Different language models may be trained using different techniques. In some implementations, the language models are “masked language models,” which are trained with a masked objective. During such training, some amino acids are replaced with “mask” tokens, and the model is trained to predict the masked amino acids, e.g., using a categorical cross entropy loss. Other implementations can use alternative training procedures, such as an autoregressive training objective that predicts the “next” token given the part of the sequence to the left of the token.

Architecturally, these language models are based on an architecture known as the transformer, but alternative architectures (e.g., recurrent neural networks with long short-term memory) could also be used.

For each language model, a sequence (e.g., wildtype) can be input into the language model. For each position in the sequence, the model outputs a likelihood value for each possible amino acid at that position. For example, given a sequence of length 100 and 20 amino acids, the model would output 2000 likelihoods.

Since the likelihoods of all amino acids can be determined, the likelihood of the wildtype residue at each position is determined. Then, at a given position, it can be determined whether any of the variant residues have a higher likelihood than the wildtype residue. For each position, all mutations (variants) in which the likelihood value of that mutation is greater than the likelihood value of the wildtype residue can be identified. We refer to this as a language model “recommending” a set of mutations.

### B. Ensemble of Language Models

Some embodiments can use an ensemble (group) of language models, with the results (prediction of which variants are likely) of each of the models being used to determine a final list of candidate variants. Various logic can be used to determine how to combine the results, e.g., a requirement that a variant (mutation) occurs in at least a specific number of models, such as 2, 3, 4, etc. (or 20%, 30%, 40%, etc.).

Accordingly, some embodiments can repeat the entire procedure described above for each language model (e.g., six language models as used herein, but other number of models can be used, such as 4, 5, 6). Then, for each mutation, the number of language models that recommend that mutation is counted. The mutations satisfying one or more criteria are identified. The one or more criteria can include the requirement mentioned above for the variant occurring in at least a specific number of models. The number of mutations identified depends on the input sequence, e.g., the wildtype sequence or a current version of an antibody being developed. As an example, a small set of mutations (e.g., 10-30) may be recommended by a threshold number of the language models (e.g., two or more language models).

Additional criteria can be used, e.g., the likelihood value can be subjected to further criteria. The absolute value of the likelihood can be required to be above a certain threshold. As another example, the difference of the mutation likelihood and the input residue likelihood can be subjected to one or more criteria, e.g., required to be above a threshold. Such a likelihood different or absolute likelihood can be ranked, and a specified number of mutations can be selected (e.g., 10, 20, 30, etc.). For instance, some embodiments can preferentially select mutations based on the number of language models that recommend a mutation. For example, if six language models recommend a mutation, we would prefer that mutation to a different mutation recommended by only two language models.

In one implementation, evolution was performed by first measuring the highest likelihood mutations recommended by a consensus of the ESM-1b and ESM-1v ensemble of five language models (to make six language models in total) [14], [15]. The ESM-1b language model differs from the ESM-1v language models in that it was trained on the UniRef50 dataset, while the five ESM-1v language models were trained on the UniRef90 dataset. The five ESM-1v language models differ from each other in that their weights were initialized to different values (e.g., different random seeds for both model weight initialization and during stochastic gradient descent), and these models were given access to training sequences in different random orders during the optimization procedure, resulting in different model weights. The wildtype sequence was input into each of the language models. These language models were used to compute likelihoods of all single-residue mutations to the variable regions of the heavy chain (VH) or the light chain (VL). Based on these mutational likelihoods, mutations were acquired with higher evolutionary likelihood than wildtype according to two or more language models. In this manner, antibody sequence with improved affinity can be identified, as opposed to a naïve library of sequences.

### C. Experimental Validation

The set of mutations satisfying the one or more criteria can be considered candidate mutations. This set of candidate mutations can be experimentally validated to identify the mutations that improve one or more desirable properties, such as binding affinity, higher neuralization, thermostability preserved or increased, and solubility. The experimental validation can involve one or more of various laboratory techniques, such as screening for binding using biolayer interferometry, neutralization activity using pseudovirus-based assays, thermostability using thermal melts, or solubility by measuring optical density.

In some implementations, validation tests were run for the candidate mutations, e.g., using sequences with a single substitution from the input sequence (e.g., wildtype). For example, in a first round of evolution, we can experimentally test the effect of each mutation individually on binding and/or other properties. From this data, we then determine the set of mutations that, individually, preserve or improve binding. Such validation could stop at this point with the single substitution mutations, but other implementations can continue the validation.

Some implementations can further explore other combinations of mutations. For example, one or more additional rounds of validation can be performed. In the second round, mutations with preserved or improved binding (affinity) based on the results of the first round were combined. Thus, combinations of two mutations can be investigated. Such a later round can compute all possible combinations of the mutations that worked well (e.g., preserved or improved the property) in the first round. For instance, all possible combinations of pairs, triples, etc. can be tested for the property (e.g., binding).

However, in cases where the number of all possible combinations is very large, a subset can be selected for additional experimental measurement. For example, to pick the subset, preferred mutations that had the best effect on binding during the first round can be selected. Even if not all combinations are tested, a good representation of different numbers of mutations can be tested (e.g., some variants with two mutations from wildtype, some with three, etc.). Accordingly, for later rounds, e.g., with 3 or more mutations, certain permutations can be selected based on the pairs of combinations that improve the property over having just one of the mutations. If none of the variants in the second round improve binding over the first round, the highest-affinity variant(s) would be from the first round

The antibody variants (e.g., above a threshold or the top N variants, e.g., top 5 or 10) with the best binding affinity (or another property) across one or more rounds (e.g., both rounds) can be followed up for additional testing. Such additional testing can include characterizing the variant's thermostability, solubility, viral neutralization activity, and potentially its ability to prevent or treat disease in an animal.

### D. Example Implementation for Language Model Consensus

An example implementation for the output of the models and selection of mutations by one or more criteria is provided below. To acquire variants for experimental measurement, mutations were selected that are recommended by a consensus of language models. A single wildtype sequence x∈ [symbol] ^{N} was taken as input, where [symbol] is the set of amino acids and N is the sequence length. A set of masked language models were selected pretrained to produce conditional likelihoods p(xᵢ|x). This likelihood is of a residue x occurring at position i, given the input sequence x.

To guide evolution based on a certain language model, the set of mutations with higher language-model likelihood than the wildtype was first computed, i.e., the set

$$ℳ ( {\hat{p}}_{a} ) {=}^{def} \{ i ∈ [ N ] , {x}_{i}^{′} ∈ : {\hat{p}}_{a} ( {x}_{i}^{′} \vert x ) > {\hat{p}}_{a} ( {x}_{i} \vert x ) \} ,$$

was computed, where \hat{p}ₐ denotes the language model and xi denotes the wildtype residue. To further filter mutations to only those with the highest likelihood, mutations were chosen based on a consensus scheme, where, for a mutation xᵢ′,

$$f ( {x}_{i}^{′} ) {=}^{def} {\sum }_{j ∈ [ M ]} \{ ( i , {x}_{i}^{′} ) is in ℳ ( {\hat{p}}_{j} ) \}$$

was computed, where [symbol] {·}denotes the indicator function and there are M language models. The set of mutations with higher likelihood than wildtype across multiple language models were then acquired, i.e.,

$$𝒜 {=}^{def} \{ i ∈ [ N ] , {x}_{i}^{′} ∈ : f ( {x}_{i}^{′} ) \geq k \}$$

was acquired, where k is a user-supplied cutoff that controls the number of corresponding variants to measure.

In an example implementation in this disclosure, six large-scale masked language models were used, namely, the ESM-1b model [14] and the five models that are ensembled together to form ESM-1v, both obtained from github.com/facebookresearch/esm. ESM-1b was trained on the March 2018 release of UniRef50 [17] consisting of ˜27 million sequences, and the five models in ESM-1v were each trained on the March 2020 release of UniRef90 [17] consisting of ˜98 million sequences. Such machine learning techniques can be performed by a computer system.

### E. Training Using Self-Supervision

The models can be trained without knowledge about the type of sequences that would necessarily increase certain properties, such as binding affinity or stability. Instead, such properties are emergent properties. In this manner, data does not have to be gathered, thereby making the training process efficient. As an example, the training set can be a UniRef dataset, such as UniRef90 and UniRef90, or an abYsis dataset.

It is surprising that an unsupervised model could predict variants that improve such physical properties mentioned herein. Instead, optimizing for evolutionary plausibility can provide proteins with improved properties.

### F. Computational Efficiency of the Approach.

Because the approach described herein is based on pretrained language models, the computational pipeline is highly efficient at making predictions, taking less than a second per antibody (including both VH and VL sequences) on widely-available, GPU-accelerated hardware (see Example 1). To demonstrate efficiency, the algorithm was evaluated on 742 therapeutically-relevant antibodies from the Thera-SAbDab database [32](data not shown). Predictions were made over all 742 antibodies in ˜3 minutes, and the approach scales linearly with the number of antibodies. This timescale is shorter by orders of magnitude than those of in-vitro assays for identifying candidate mutations (for example, via cell-surface display).

### G. Use of Structural Information

In previous sections, we described the ability of an ensemble of natural language models trained on evolutionary-scale protein sequences to successfully predict beneficial mutations in human antibodies, thereby improving the critical properties of antibody binding affinity and viral neutralization. Importantly, we showed that this optimization method can be applied to any therapeutic antibody from its protein sequence alone.

In this section, we describe an enhanced method to engineer optimized therapeutic antibodies when both sequence and structural information are available for the antibody-antigen pair using a single computational model. Accordingly, in some implementations, the only input into the algorithm may be the amino acid sequence of the protein. In other implementations, three-dimensional information about the protein can also be used.

The three-dimensional information can be used via an inverse folding model application of language models. Accordingly, in addition to sequence, the computer can receive structural information, e.g., the three-dimensional coordinates of the backbone atoms that correspond to those positions. As examples, the training data of the three-dimensional coordinates can be existing structures in the Protein Data Bank (wwpdb.org/) and AlphaFold structures (alphafold.ebi.ac.uk/), which may be predictions made by another model. For a given structure, a probability for a given sequence to fold into that structure can be determined using the machine learning language model.

For embodiments involving antibodies binding to antigens, the sequence and/or three-dimensional structure of the antigen (e.g., a coronavirus surface protein) can be used. Thus, some embodiments can use the sequence of the antibody, the backbone structure of the antibody, the sequence of the antigen, and the backbone structure of the antigen.

In some implementations, the structural information can be input to a precursor layer (e.g., one or more geometric vector perceptron layers) that transforms the coordinates for input to layers of a language model. The three-dimensional (3D) coordinates can be mapped to some internal representation, which can be invariant to translations or rotations. The protein sequence and the transformed coordinates (or directly the structural information) can be concatenated for input to the language model. Accordingly, instead of a just a language model of sequences of amino acids, the language model can receive a sequence of tokens (representing the amino acids) concatenated with features that are rotational invariant from the input of the structural information.

As with using just the evolutionary sequence, candidate sequence with a high likelihood of forming into the target structure can be identified and experimentally validated. Candidate sequences, each of which differ from the original antibody sequence by only one amino acid, are then selected based on ranked-order of log-likelihood scores from the deep mutational scan library to be cloned as recombinant DNA, expressed in mammalian cells, purified, and screened using biolayer interferometry (BLI) and pseudotyped lentivirus to determine the antibody affinity and neutralization capacity, respectively, of the newly designed mutated protein. Single amino acid mutations from this candidate sequence list that are experimentally validated to enhance viral neutralization can then selected for a subsequent round of evolution in which these experimentally validated mutations are combined together to be screened for possible additive or synergistic effects. Such experimental methods can be identical to those described in elsewhere in this disclosure.

### 1. Inverse Folding (Graph Neural Networks)

For a given structure, an inverse folding aspect of a machine learning language model can predict the probability for a given sequence(s) to fold into the given structure (potentially including a complex of two or more protein chains). The structure could correspond to a known structure for the sequence or a structure complex for binding to an epitope, e.g., of an antigen.

In some embodiments, to accomplish this task, we utilize an autoregressive inverse folding model that scores the likelihood of an input candidate sequence(s) folding into an input fixed-backbone protein design (e.g., of antibody-antigen interface). Such a model can be trained over a data set of protein structures (e.g., millions of structures), which may be composed of computationally predicted structures of the UniRef50 sequence library.

The model's architecture can implement a precursor layer of a graph neural network for processing the structural information. The graph neural network can include one or more geometric vector perceptron (GVP) layers that extract geometric features invariant to rotations of the input protein molecule backbone. The output of the precursor layer can be concatenated together with one or more protein sequences. The concatenated input can be used for the language modeling described above.

Accordingly, following invariant geometric input processing, the vector features can be supplied to a generic autoregressive sequence-to-sequence encoder-decoder transformer [Hsu C, Verkuil R, Liu J, et al. Learning inverse folding from millions of predicted structures. bioRxiv; 2022. DOI: 10.1101/2022.04.10.487779]. While the data used to train the model can be composed of single chain proteins, we demonstrate that it can be used to forward engineer higher affinity interactions in a protein complex, specifically an antigen-antibody complex which consists of at least three protein chains (2 from the antibody and >1 from the antigen).

The model can provide a log-likelihood score for every candidate sequence (or just some) of a deep mutational scan of the target antibody's variable region. The model can also be provided with the of the backbone coordinates from an experimentally solved structure (either using X-ray crystallography or cryo-electron microscopy) of the fragment antigen-binding region (Fab) in complex with its antigen or a computationally predicted structure.

The graph neural network used in the Hsu et al is further described in Jing, Bowen, et al. “Learning from Protein Structure with Geometric Vector Perceptrons.” International Conference on Learning Representations. 2020 (arXiv preprint arXiv:2009.01411). Further details can be found in Hsu et al. and Jing et al.

In some implementations, the backbone structure can be represented as a graph where each node corresponds to an amino acid and has embedding with the following features: scalar features can correspond to angles between nodes; forward and reverse unit vectors in the directions of neighboring nodes; the unit vector in the imputed direction for side chains, which can be computed by assuming tetrahedral geometry and normalizing—the unit vectors can unambiguously define the orientation of each amino acid residue; and a one-hot representation of amino acid identity. The model can also include one last GVP layer (e.g., with a 20-way scalar softmax output) to predict the probability of the amino acids.

Further details for the inverse folding model description are provided. As input to the inverse folding model, we have a protein structure Y∈ [symbol] ^{N×3×3}, where N is the number of amino acids, and each amino acid is featurized by three atoms (the alpha-carbon, beta-carbon, and nitrogen atoms in the protein backbone) and where each atom has three dimensions in physical space (hence the dimensionality N×3×3). Our inverse folding model learns the probability distribution p of a protein sequence x=(x₁, . . . , x_{N})∈ [symbol] ^{N} (where [symbol] is the alphabet of amino acids) given a structure Y via the chain rule of probability, i.e.,

$$p ( x \vert Y ) = p ( {x}_{1} \vert Y ) p ( {x}_{2} \vert {x}_{1} , Y ) … p ( {x}_{N} \vert {x}_{1} , … , {x}_{N - 1} , Y ) .$$

The structure Y can correspond to the structure of interest, which may be a binding interface or a known structure of a protein.

Embodiments can implement p with a neural network that takes as input both the entire backbone structure Y and the left-sequence context (x₁, . . . , xᵢ₋₁); i.e., the neural network model will learn the probability distribution

$$p ( {x}_{i} \vert {x}_{1} , … , {x}_{i - 1} , Y ) .$$

This probability distribution can be defined over the alphabet [symbol] , so it is a 20-dimensional vector (since there are 20 amino acids) and all entries in the vector sum to 1. A probability vector can be provided at each position. By learning the probability of a single site given the left sequence context, then via the chain rule of probability, this can also define the probability of an entire sequence (i.e., by multiplying N of these terms together).

Further details for scoring sequences via inverse folding are provided. Assume we now have some known sequence \hat{x}=(\hat{x}₁, . . . , \hat{x}_{N}) and its corresponding given structure Y. We can score how “good” \hat{x} is under the inverse folding model p by computing the value of p(x=\hat{x}|Y), which we can do autoregressively as

$$p ( x = \hat{x} \vert Y ) = p ( {x}_{1} = {\hat{x}}_{1} \vert Y ) … p ( {x}_{N} = {\hat{x}}_{N} \vert {\hat{x}}_{1} , … , {\hat{x}}_{N - 1} , Y ) .$$

This will give us a probability score between 0 and 1, inclusive. Note that one can evaluate the inverse folding model N times, starting with p(x₁=\hat{x}1|Y) (probability of the first amino acid being x₁ for the given structure Y) and ending with p(x_{N}=\hat{x}_{N}|, . . . , \hat{x}_{N-1}, Y). On each evaluation, we compute the probability at site i given the full structure and all amino acids in the sequence from site 1 to site i−1, i.e., the “left-sequence context.” The probability is of a sequence x occurring within the given structure Y. For a given structure Y and two sequences xa and xb, where xa is a natural sequence that does fold into a structure that resembles Y and xb is some random sequence, the probability for p(xa|Y) would be much higher than p(xb|Y).

Once we compute the score p(x=\hat{x}|Y), we use that score as the prediction for “fitness” (e.g., binding affinity, enzymatic activity, folding stability). Via the chain rule, the probability of the full sequence can be computed by multiplying those N probability terms. The output is the probability of seeing a sequence x given a structure Y. There can be one structure Y and many (e.g., 1000) sequences x. We can then compute a score for each of those sequences given the same structure. In some implementations, the inverse folding model does not have any explicit access to “fitness” during either training or evaluation, which we refer to as “zero shot” fitness prediction.

The use of the inverse folding can be considered as a “structure conditioned language model” in that the model is still doing language modeling, but the language model also has access in its input to the entire structure Y, so where regular autoregressive language modeling learns p(x₁)p(x₂|x₁) . . . p(x_{N}|x₁, . . . , x_{N-1}), inverse folding learns instead p(x₁|Y)p(x₂|x₁, Y) . . . p(x_{N}|x₁, . . . , x_{N-1}, Y)

### 2. Binding Affinity Prediction and Scoring Multichain

In some embodiments, the target structure can correspond to a binding interface between a first protein (e.g., an antibody) and a second protein (e.g., an antigen). A sequence of amino acids can be sampled to identify sequences that are likely to fold into the three-dimensional target structure. If the structure is similar to what is expected for binding, then that sequence might bind well. For example, the epitope of an antigen can be used in the inverse folding scheme to see if there is a high probability for a predicted sequence.

Accordingly, if one has the backbone coordinates of a binding interface, the model can inverse fold the binding interface. If a protein sequence (e.g., antibody and antigen sequence) has high likelihood of forming that interface, then those sequences are more likely to form a strong binding interaction. In this manner, we can use the model to engineer new proteins and validate them in the laboratory.

As described above, the language modeling can work within a graph neural network feature as an auto-regressive model. The input can include the whole set of backbone coordinates, and then proceed to do left to right filling of the sequence into that backbone structure. The language modeling can fill in the sequence given the entire structure. A sequence that would likely occur can be identified and that is going to bind to the antigen. The binding affinity would depend on the actual sequence as well as the probability of forming the structure.

Multichain analysis can be performed. When the antigen information is provided, embodiments can treat the two or more chains in a complex as a single protein and identify what amino acids are likely to occur in that complex. If the sequences are likely to occur in the antibody antigen complex, there is a higher binding affinity.

An example for scoring multichain sequences via inverse folding is provided. For antibody-antigen interactions, for example, we can extend this model to handle multiple chains by simply concatenating sequences and their respective structures. For example, if we have three sequences x_{Ab heavy chain}, x_{Ab light chain}, and x_{antigen} (as well as corresponding respective structures Y_{Ab heavy chain}, Y_{Ab light chain}, and Y_{antigen}), we can simply define

$${x}_{all} {=}^{def} [ \begin{matrix}{x}_{Ab heavy chain} \\ {x}_{Ab light chain} \\ {x}_{antigen}\end{matrix} ] and {Y}_{all} {=}^{def} [ \begin{matrix}{Y}_{Ab heavy chain} \\ {Y}_{Ab light chain} \\ {Y}_{antigen}\end{matrix} ] .$$

Then, we can compute the score p(xₐₗₗ|Yₐₗₗ), which we use as our zero-shot prediction score. In practice, we can optionally add special tokens that tell the model where each chain begins, but this does not change the conceptual idea of simply concatenating xs and Ys to handle multichain scoring. Accordingly, for multichain, the sequences can be stacked together. In the input to the model, one can concatenate them. There can be input separator tokens in between the sequences indicating a new chain has started.
3. Stabilization of 3D structure

Another application involves isolated proteins in which case it is not an interface but the stabilization of the protein per se. An example of such an application is prediction of amino acid substitutions that stabilize the protein, e.g., SARS-CoV-2 spike protein. Such an analysis can use the three-dimensional information of the spike trimer, but there is no antigen. A multi-chain analysis can also apply in such an example. Embodiments can predict substitutions that might be useful to stabilize that protein, e.g., for future vaccine use.

In this work, we demonstrate the effectiveness this novel framework of computationally evolving antibodies by selecting mutations that are computationally predicted to be more to likely fold into the experimentally solved structure than the native sequence itself by attempting to co-evolve therapeutic antibodies against SARS-CoV-2 against emerging viral variants which themselves have evolved under selective pressure to escape humoral response. The antibody named LY-1404 (Bebtelovimab) was recently discontinued by the FDA citing lack of susceptibility to the drug by the BQ.1.1 viral variant. In the provided data (Table 14) we show that our method yields a series of candidate mutations to LY-1404 that improve its binding and viral neutralization against this variant. Similarly, we provide a list of candidate mutations to the therapeutic antibody SA58 which is currently in clinical trials, that improve its binding affinity to SARS-CoV-2 variants BQ.1.1, XBB, and XBB.1.5 (subvariants of B.1.1.529 (omicron variant of SARS-CoV-2) (Table 15).

Accordingly, some embodiments can be applied to engineer antibodies for which there exists an experimentally solved structure of the antibody in complex with the antigen. Other embodiments can perform the optimization with only the sequence and structure of the antibody, and not the antigen.

### H. Method for Identifying Candidate Mutations

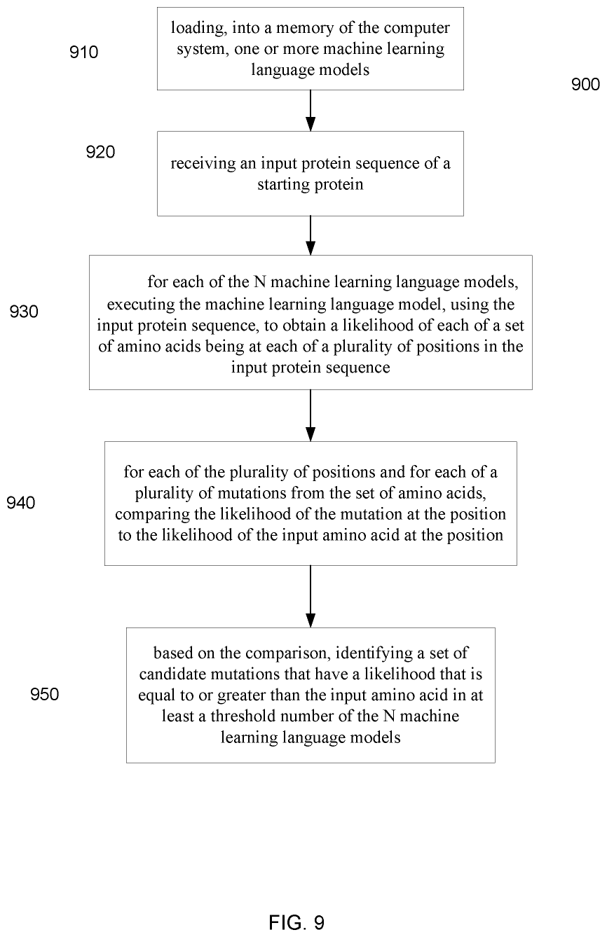

FIG. 9 is a flowchart illustrating a method of using machine learning language model(s) to identify candidate mutations. The method may be performed by a computer system. Additional steps can be controlled by the computer system or performed independently using other devices, e.g., measurement techniques using one or more other machines controlled by a person.

In step 910, N machine learning language models can be loaded into a memory of the computer system. For example, a trained machine learning language model can be loaded into RAM. N is an integer equal to or greater than 1, including, 2, 3, 4, 5, 6, 7, and so on. Examples of language models are n-grams, exponential, and neural networks, e.g., recurrent neural networks or transformers. A language model can provide a probability distribution over sequences of tokens, e.g., words or amino acids. As described above, given any sequence of words of length m, a language model can assign a probability P(w₁, . . . , wₘ) to the whole sequence. The N machine learning language models can be trained on one million or more protein sequences that occur in nature.

In step 920, the computer system receives an input protein sequence (e.g., an antibody sequence, which may be part of an antibody) of a starting protein. The input protein sequence is comprised of input amino acids. The input protein sequence can be received in various ways, e.g., read from a file, which may be on a network or local, or received from user input, such as a keyboard. Example lengths of the input protein sequence can be from the entire protein or from just the variable reasons. As examples, the lengths of the sequence can be equal to or greater than 50, 60, 70, 80, 90, 100, 120, 140, 160, 180, 200, 250, 300, 350, 400, 450, and 500 amino acids. The input protein sequence can be a wildtype sequence. As examples, the protein can be an enzyme or an antibody.

In addition to receiving a protein sequence, structural information can be received, as described above. Thus, structural information for a target protein structure can be received. The target protein structure can be a complex including an interface for a resulting protein to bind to an antigen.

Steps 930 and 940 can be performed for each of the N machine learning language models.

In step 930, the computer system can execute the machine learning language model, using the input protein sequence, to obtain a likelihood of each of a set of amino acids (e.g., all 20 human amino acids) being at each of a plurality of positions in the input protein sequence. Examples of such a likelihood value is described above. The plurality of positions can be all the positions of the input protein sequence or just a portion of the positions. The likelihood can be determined based on changing just the amino acids at the current position being analyzed.

For implementations using structural information, the structural information for the target protein structure can be input to the machine learning language model. When the target protein structure is a complex including a protein (e.g., an antibody and an antigen), the antigen sequence and the antigen structure can be input to the machine learning language model. The target protein structure can include a heavy chain and a light chain of an antibody. The resulting protein sequence can include a heavy chain sequence and a light chain sequence.

The output of the machine learning language model comprises the likelihood that includes the probability that the target protein structure is formed of the resulting protein sequence. The likelihood of the mutation being at the position can include a probability that the target protein structure (possibly being a complex) is formed of a resulting protein sequence that includes the mutation at the position. For example, when a different amino acid is placed at a position 5 (or other position) forming a mutated sequence, a probability of the target protein structure being formed from the mutated sequence.

In step 940, for each of the plurality of positions and for each of a plurality of mutations from the set of amino acids, the likelihood of the mutation at the position can be compared to the likelihood of the input amino acid at the position. The set of amino acids can include various numbers of amino acids, e.g., 10, 11, 12, 13, 14, 15, 16, 17, 18, 19, 20, or more than 20. The set of amino acids can be all 20 amino acids that comprise proteins in a human. Thus, if there are 100 positions and 20 amino acids being tested, then at each position the 19 other amino acids can be tested by comparing the likelihood of the other amino acid to the likelihood of the amino acid in the input amino acid.

In step 950, based on the comparison, a set of candidate mutations can be identified that have a likelihood that is equal to or greater than the input amino acid in at least a threshold number of the N machine learning language models. Such example thresholds are provided above, e.g., 1, 2, 3, 4, 5, and so on, and may be specified as a percentage of N.

After the set of candidate mutations is identified, experimental validation can be performed, as described herein. For example, for each candidate mutation of the set of candidate mutation, a mutated protein having the mutation can be created. Techniques to create the mutated protein are known to the skilled person. The mutated protein can be experimentally tested to determine whether the mutated protein has a same or improved property relative to the starting protein. In this manner, a first set of validated mutated proteins (e.g., antibodies) having validated mutations can be identified. Examples of the property are provided above and can include a binding affinity to a target molecule.

After an initial round of validation, one or more additional rounds can be performed to test sequences with multiple mutations. For example, for each of the validated mutations, multiple-mutated proteins having a plurality of the validated mutations can be created. The multiple-mutated protein can be experimentally tested to determine whether the mutated protein has a same or improved property relative to the starting protein or to any single-mutated protein. In this manner, a second set of validated multiple-mutated antibodies having validated multiple-mutations can be identified.

More than one property can be tested, e.g., after the validated mutations are identified using a first property. Thus, additional testing of the first set of validated mutated proteins or the second set of validated multiple-mutated proteins for additional properties can be performed.

### I. Example Computer System

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems of the computer can be interconnected via a system bus. Additional subsystems such as a printer, keyboard, storage device(s), monitor (e.g., a display screen, such as an LED), which is coupled to display adapter, and others are shown. Peripherals and input/output (I/O) devices, which couple to I/O controller, can be connected to the computer system by any number of means known in the art such as input/output (I/O) port (e.g., USB, FireWire®). For example, I/O port or external interface (e.g., Ethernet, Wi-Fi, etc.) can be used to connect computer system 10 to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) may embody a computer readable medium. Another subsystem is a data collection device, such as a camera, microphone, accelerometer, and the like. Any of the data mentioned herein can be output from one component to another component and can be output to the user.

A computer system can include a plurality of the same components or subsystems, e.g., connected together by external interface, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

The following examples are offered to illustrate, but not to limit the claimed invention.

### Example 1. Materials and Methods Used in the Examples

Antibody sequence analysis and evolution. For antibodies, the above steps were performed for the VH and VL sequences separately, obtaining respective sets [symbol] _{VH} and [symbol] _{VL}. [symbol] _{VH} is the set of amino acids that were acquired from the ensemble's predictions for the heavy chain, and likewise for [symbol] _{VL}, for the light chain. For round 1 of evolution, values of k were chosen such that | [symbol] _{VH} ∪ [symbol] _{VL}| is approximately 10, which is meant to be a reasonable number of antibody variants for one person to express and purify in parallel. The value k is the cutoff controlling the number of consensus language models used to determine the predictions that were acquired. A is the set of language-model-recommended variants. k is used as a parameter to control A. Having higher k value increases the stringency of acquired variants, and thereby corresponds to fewer variants. k=3 was used for MEDI8852 VH and VL, k=2 for MEDI8852 UCA VH and VL, k=4 for mAb114 VH and VL, k=2 for mAb114 UCA VH and VL, k=2 for S309 VH and VL, k=2 for REGN10987 VH and VL, k=2 for C143 VH and VL. For round 2 of evolution, variants were first measured for binding affinity to a given antigen via BLI (more details below) and those that preserve or enhance affinity were recombined such that the second-round variants have two or more mutations from wildtype. For MEDI8852 and MEDI8852 UCA, all possible combinations were tested; for the other antibodies, where the number of possible combinations far exceeds the ˜10-variant limit, a set of combinations meant to prioritize inclusion of mutations that resulted in the largest improvements in affinity during the first round were manually selected.

The wildtype sequences provided by the original study authors describing the respective antibodies were used. Wildtype VH and VL sequences are provided as SEQ ID NOs: 1-14 and 58-61 herein. The Kabat region definition provided by the abYsis webtool version 3.4.1 (abysis.org/abysis/index.html) [30] was used to annotate the framework regions and CDRs within the VH and VL sequences (Table 1).

Antibody cloning. The antibody sequences were cloned into the CMV/R plasmid backbone for expression under a CMV promoter. The heavy chain or light chain sequence was cloned between the CMV promoter and the bGH poly(A) signal sequence of the CMV/R plasmid to facilitate improved protein expression. Variable regions were cloned into the human IgG1 backbone; REGN10987 and C143 variants were cloned with a lambda light chain, while variants of all other antibodies were cloned with a kappa light chain. The vector for both heavy and light chain sequences also contained the HVM06_Mouse (P01750) Ig heavy chain V region 102 signal peptide to allow for protein secretion and purification from the supernatant. VH and VL segments were ordered as gene blocks from Integrated DNA Technologies and were cloned into linearized CMV/R backbones with 5× In-Fusion HD enzyme premix (Takara Bio).

Antigen cloning. HA, GP, Spike, and RBD sequences were cloned into a pADD2 vector between the rBeta-globin intron and β-globin poly(A). HA constructs contained a Foldon trimerization domain. GP and Spike constructs contained a GCN4 trimerization domain. All HAs, GP, Wuhan-Hu-1 S-6P, Omicron, BQ.1.1, RBD constructs contained an AviTag. All constructs contained a C-terminal 6×His tag. HA sequences from the following strains were used: A/New Caledonia/20/1999(H1N1) (H1 Caledonia), A/Solomon Islands/3/2006(H1N1) (H1 Solomon), A/Japan/305/1957(H2N2) (H2 Japan), A/Panama/2007/1999(H3N2) (H3 Panama), A/Victoria/3/1975(H3N2) (H3 Victoria), A/swine/Hubei/06/2009(H4N1) (H4 Hubei), A/Vietnam/1203/2004(H5N1) (H5 Vietnam), A/Hong Kong/61/2016(H7N9) (H7 HK16), and A/Hong Kong/I25/2017(H7N9) (H7 HK17). Ebola GP ectodomain (Mayinga, Zaire, 1976, GenBank: AAG40168.1) with the mucin-like domain deleted (A309-489) was used. Spike or RBD sequences were based off wildtype Wuhan-Hu-1 (GenBank: BCN86353.1), Beta (GenBank: QUT64557.1), or Omicron (GenBank: UF069279.1).

DNA preparation. Plasmids were transformed into Stellar competent cells (Takara Bio), and transformed cells were grown at 37° C. Colonies were sequence confirmed and then maxi-prepped per the manufacturer's recommendations (NucleoBond Xtra Maxi; Macherey-Nagel). Plasmids were sterile filtered using a 0.22-μm syringe filter and stored at 4° C.

Protein expression. All proteins were expressed in Expi293F cells. For proteins containing a biotinylation tag (AviTag), Expi293F cells containing a stable BirA enzyme insertion were used, resulting in spontaneous biotinylation during protein expression. Expi293F cells were cultured in media containing 66% Freestyle/33% Expi media (ThermoFisher) and grown in TriForest polycarbonate shaking flasks at 37° C. in 8% carbon dioxide (CO₂). The day before transfection, cells were spun down and resuspended to a density of 3×10⁶ cells/mL in fresh media. The following day, cells were diluted and transfected at a density of approximately 3-4×10⁶ cells/mL. Transfection mixtures were made by adding the following components: maxi-prepped DNA, culture media, and FectoPro (Polyplus) would be added to cells to a ratio of 0.5 μg:100 μL:1.3 μL:900 μL. For example, for a 100 mL transfection, 50 g of DNA would be added to 10 mL of culture media, followed by the addition of 130 uL of FectoPro. For antibodies, the transfection DNA was divided equally among heavy- and light-chains; in the previous example, 25 g of heavy-chain DNA and 25 g of light-chain DNA would be added to 10 mL of culture media. Following mixing and a 10-minute incubation, the example transfection cocktail would be added to 90 mL of cells. The cells were harvested 3-5 days post-transfection by spinning the cultures at >7,000× g for 15 minutes. Supernatants were filtered using a 0.45-μm filter.

Antibody purification. Antibodies were purified using a 5 mL MAb Select Sure PRISM™ column on the AKTA pure FPLC (Cytiva). The AKTA system was equilibrated with line A1 in 1× phosphate-buffered saline (PBS), line A2 in 100 mM glycine pH 2.8, line B1 in 0.5 M sodium hydroxide, Buffer line in 1×PBS, and Sample lines in water. The protocol washes the column with A1, followed by loading of the sample in the Sample line until air is detected in the air sensor of the sample pumps, followed by 5 column volume washes with A1, elution of the sample by flowing of 20 mL of A2 (directly into a 50 mL conical containing 2 mL of 1M Tris pH 8.0) followed by 5 column volumes of A1, B1, and A1. The eluted samples were concentrated using 50 or 100 kDa cutoff centrifugal concentrators followed by buffer exchange using a PD-10 column (SEPHADEX) that had been preequilibrated into 1×PBS. Purified antibodies were stored at −20° C.

Antigen purification. All antigens were His-tagged and purified using HisPur™ Ni-NTA resin (ThermoFisher). Cell supernatants were diluted with ⅓rd volume wash buffer (20 mM imidazole, 20 mM HEPES pH 7.4, 150 mM NaCl or 20 mM imidazole, 1×PBS) and the Ni-NTA resin was added to diluted cell supernatants. For all antigens except SARS-CoV-2 Spike, the samples were then incubated at 4° C. while stirring overnight. SARS-CoV-2 Spike antigens were incubated at room temperature. Resin/supernatant mixtures were added to chromatography columns for gravity flow purification. The resin in the column was washed with wash buffer (20 mM imidazole, 20 mM HEPES pH 7.4, 150 mM NaCl or 20 mM imidazole, 1×PBS) and the proteins were eluted with either 250 mM imidazole, 20 mM HEPES pH 7.4, 105 mM NaCl or 20 mM imidazole, 1×PBS. Column elutions were concentrated using centrifugal concentrators at 50 or 100 kDa cutoffs, followed by size-exclusion chromatography on an AKTA Pure system (Cytiva). AKTA pure FPLC with a Superdex 6 Increase (S6) or Superdex 200 Increase (S200) gel filtration column was used for purification. 1 mL of sample was injected using a 2 mL loop and run over the S6 or S200 which had been preequilibrated in degassed 20 mM HEPES, 150 mM NaCl or 1×PBS prior to use and stored at −20° C.

Fab production and purification. 1/10 volume of 1M Tris, pH 8 was added to IgGs at −2 mg/mL in 1×PBS. 2 μL of a 1 mg/mL stock of Lys-C(stock stored at −20° C.) was added for each mg of human IgG1 and digested for 1 hour at 37° C. with moderate rotation. Digested Fabs were purified by SP/AKTA using 50 mM NaOAc, pH 5.0 with gradient NaCl elution (using 50 mM NaOAc+1M NaCl, pH 5.0). Fab fractions were pooled and dialyzed against 1×PBS and concentrated using 30 kDa concentrators. Purified Fabs were stored at −20° C.

Biolayer interferometry (BLI) binding experiments. All reactions were run on an Octet Red 96 and samples were run in 1×PBS with 0.1% BSA and 0.05% Tween 20 (octet buffer). IgGs and Fabs were assessed for binding to biotinylated antigens using streptavidin (SA) biosensors (Sartorius/ForteBio) or to unbiotinylated, His-tagged antigens using Anti-Penta-HIS biosensors (Sartorius/ForteBio). Antigen was loaded to a threshold of 1 nm shift. Tips were then washed and baselined in wells containing only octet buffer. Samples were then associated in wells containing IgG or Fab at 100 nM or 1000 nM concentration. A control well with loaded antigen but that was associated in a well containing only 200 μL octet buffer was used as a baseline subtraction for data analysis. Association and dissociation binding curves were fit in Octet System Data Analysis Software version 9.0.0.15 using a 1:2 bivalent model for IgGs and a 1:1 model for Fabs to determine K_{d}. Averages and standard deviations of fitted K_{d}s from at least two independent experiments were determined (data not shown). Wildtype and the highest-affinity variants were also tested at multiple concentrations (data not shown).

Thermal melts. Thermal melting profiles of proteins were measured by differential scanning fluorimetry on a Prometheus NT.48 instrument. Protein samples (0.1 mg/mL) were loaded into glass capillaries and then subject to a temperature gradient from 20 to 95° C. Intrinsic fluorescence (350 nm and 330 nm) was recorded as a function of temperature. Thermal melting curves were plotted using the first derivative of the ratio (350 nm/330 nm). Melting temperatures were calculated automatically by the instrument and represented peaks in the thermal melting curves.

Lentivirus production. SARS-CoV-2 Spike (Wunan, BQ.1.1, BA.1, D614G and Beta variants) pseudotyped lentiviral particles were produced. Viral transfections were done in HEK293T cells using calcium phosphate transfection reagent. Six million cells were seeded in D10 media (Dulbecco's Modified Eagle Medium (DMEM)+additives: 10% fetal bovine serum (FBS), L-glutamate, penicillin, streptomycin, and 10 mM HEPES) in 10 cm plates one day prior to transfection. A five-plasmid system was used for viral production, as described in Crawford et al. [46]. The Spike vector contained the 21 amino acid truncated form of the SARS-CoV-2 Spike sequence from the Wuhan-Hu-1 strain of SARS-CoV-2 or VOCs. VOCs were based off wildtype (GenBank: BCN86353.1), Beta (GenBank: QUT64557.1), BA.1 (GenBank: OL672836.1), BQ.1.1 (GenBank: OP412163.1), or XBB.1.5 (GenBank: OP790748.1). The other viral plasmids, used as previously described [46], are pHAGE-Luc2-IRS-ZsGreen (NR-52516), HDM-Hgpm2 (NR-52517), pRC-CMV-Rev1b (NR-52519), and HDM-tat1b (NR-52518). These plasmids were added to D10 medium in the following ratios: 10 g pHAGE-Luc2-IRS-ZsGreen, 3.4 g FL Spike, 2.2 g HDM-Hgpm2, 2.2 g HDM-Tat1b, 2.2 g pRC-CMV-Rev1b in a final volume of 1000 μL.

Ebola GP-pseudotyped lentiviruses were produced using the same packaging (pHAGE-Luc2-IRS-ZsGreen) and helper plasmids (HDM-Hgpm2, HDM-Tat1b, pRC-CMV-Rev1b) but with the plasmid encoding full-length Ebola GP (GenBank: AAG40168.1).

After adding plasmids to medium, 30 L BioT (BioLand) was added to form transfection complexes. Transfection reactions were incubated for 10 minutes at room temperature, and then 9 mL of medium was added slowly. The resultant 10 mL was added to plated HEK cells from which the medium had been removed. Culture medium was removed 24 hours post-transfection and replaced with fresh D10 medium. Viral supernatants were harvested 72 hours post-transfection by spinning at 300× g for five minutes followed by filtering through a 0.45-μm filter. Viral stocks were aliquoted and stored at −80° C. until further use.

Pseudovirus neutralization. The target cells used for infection in SARS-CoV-2 pseudovirus neutralization assays were from a HeLa cell line stably overexpressing human angiotensin-converting enzyme 2 (ACE2), as well as the protease known to process SARS-CoV-2, transmembrane serine protease 2 (TMPRSS2). Production of this cell line is described in detail by Rogers et al. [47], with the addition of stable TMPRSS2 incorporation. ACE2/TMPRSS2/HeLa cells were plated one day prior to infection at 5,000 cells per well. For Ebola-pseudovirus neutralization assays, HEK-293T cells were seeded in 96-well plates one day prior to infection at 20,000 cells per well. 96 well white walled, white bottom plates were used for neutralization assays (Thermo Fisher Scientific).

On the day of the assay, purified IgGs in 1×PBS were sterile filtered using a 0.22-μm filter. Dilutions of this filtered stock were made into sterile 1×DPBS (Thermo Fisher Scientific) which was 5% by volume D10 medium. Samples were run in technical quadruplicate in each experiment. A virus mixture was made containing the virus of interest (for example SARS-CoV-2) and D10 media (DMEM+additives: 10% FBS, L-glutamate, penicillin, streptomycin, and 10 mM HEPES). Virus dilutions into media were selected such that a suitable signal would be obtained in the virus-only wells. A suitable signal was selected such that the virus only wells would achieve a luminescence of at least >5,000,000 RLU. 90 L of this virus mixture was added to each of the antibody dilutions to make a final volume of 120 μL in each well. Virus only wells were made which contained 30 μL 1×DPBS and 90 L virus mixture. Cells-only wells were made which contained 30 μL 1×DPBS and 90 μL D10 media.

The antibody/virus mixture was left to incubate for 1 hour at 37° C. Following incubation, the medium was removed from the cells on the plates made 1 day prior. This was replaced with 100 μL of antibody/virus dilutions and incubated at 37° C. for approximately 24 hours. Infectivity readout was performed by measuring luciferase levels. SARS-CoV-2 and Ebola pseudovirus neutralization assays were read out 48- and 72-hour post-infection, respectively. Medium was removed from all wells and cells were lysed by the addition of 100 μL BriteLite™ assay readout solution (Perkin Elmer) into each well. Luminescence values were measured using an Infinite® 200 PRO Microplate Reader (Tecan). Each plate was normalized by averaging the cells-only (0% infectivity) and virus-only (100% infectivity) wells. The neutcurve Python package version 0.5.7 was used to fit the normalized values and compute IC₅₀s.

Mutational frequency computation. The frequency of residues involved in affinity-enhancing mutations were computed by aligning the wildtype VH and VL sequences of the antibodies used to databases of protein sequences. The first database considered was UniRef90, where the same database release used to train ESM-1v was used. For each antibody protein sequence, the set of sequences in the database with 50% or higher sequence similarity was first obtained; sequence similarity was computed using the fuzzywuzzy Python package version 0.18.0. Mafft version 7.475 was then used to perform multiple sequence alignment among the set of sequences. The alignment was used to compute amino acid frequencies at each site in the VH or VL sequence. The second database considered was provided by the abYsis webtool. VH and VL protein sequences were aligned using the default settings provided in the “Annotate” tool, using the database of “All” sequences as of Mar. 1, 2022.

Therapeutic antibody database evaluation and runtime benchmark. 742 therapeutically-relevant antibodies were downloaded from the Thera-SAbDab database as of Feb. 26, 2022 (opig.stats.ox.ac.uk/webapps/newsabdab/therasabdab/) [32]. For each antibody VH and VL sequence, the same procedure described above for computing consensus mutations that have higher language-model likelihood than wildtype was used. The computational runtime was measured using the time module in Python 3.8. Experiments were performed with an Advanced Micro Devices EPYC Rome 7502P 2.5 Ghz CPU and an Nvidia Ampere A40 48 Gb GPU.

Natural protein evaluation based on scanning mutagenesis data. The ability for the language models and algorithms used in the study to guide efficient evolution in other settings beyond antibodies was evaluated. Deep mutational scanning (DMS) datasets were utilized to validate that the approach herein would enable a researcher to acquire high-fitness variants. All DMS datasets from the benchmarking study by Livesey and Marsh [35] with 90% or higher coverage of the full, single-residue mutational space were used; variants that were not measured were treated as having low fitness. A scanning mutagenesis dataset generated by Markin et al. [41] that measured Michaelis-Menten kinetics of all single-site glycine or valine substitutions to the bacterial enzyme PafA was also used; for this dataset, any language-model-recommended mutations that did not involve glycine or valine substitutions were excluded from the analysis. A cutoff was applied for each dataset to binarize sequences as high- or low-fitness variants (Table 15); enrichment of high-fitness variants was then compared among the language-model-recommended variants to the background frequency of high-fitness variants among the full mutational space. For these proteins, as with the antibody experiments, values of k that result in a small number (˜10¹) of acquired mutations were chosen: k=2 was used for ADRB2, Env, HA H1, HA H3, β-lactamase, and P53, and k=1 was used for all other proteins. To quantify the statistical significance of an enrichment, it was assumed that the null distribution of the number of high-fitness, language-model-recommended variants was given by a hypergeometric distribution, which was used to compute a one-sided P value. The hypergeometric calculator at stattrek.com/online-calculator/hypergeometric.aspx was used.

Data availability. Raw data for this study has been deposited to Zenodo at DOI:10.5281/zenodo.6415457.

Code availability. Code and scripts used in this study will be made available in a public GitHub repository upon publication.

### Example 2. Efficient Affinity Maturation with General Protein Language Models

It was hypothesized that the predictive capabilities of protein language models might enable a researcher to provide only a single, wildtype antibody sequence to the algorithm and receive a small (˜10¹) set of high-likelihood variants to experimentally measure for desirable properties like improved binding affinity. This is a very general setting that does not assume knowledge of protein structure or antigen-specific training data, thereby avoiding the resource- and time-intensive processes associated with structure determination or high-throughput screens. A major question, however, is if higher evolutionary likelihood would efficiently translate to higher fitness (defined, in this case, as higher binding affinity).

The hypothesis was tested by running separate directed evolution campaigns, guided by language-model likelihood, for nine antibodies representing diverse antigens and degrees of maturity. These antibodies are:
MEDI8852: A broadly-neutralizing antibody (bnAb) that binds influenza A hemagglutinin (HA) across Group 1 and Group 2 variants and that reached Phase-II clinical trials; this antibody is highly matured, with its parent being isolated from a human followed by substantial artificial evolution [22].
MEDI8852: unmutated common ancestor (UCA): The unmatured, inferred germline sequence of MEDI8852, which only neutralizes viruses with Group 1 HAs [22].
mAb 114: A patient-derived antibody that neutralizes ebolavirus by binding to its glycoprotein (GP) and has approval for clinical use by the United States Food and Drug Administration (FDA) [23].
mAb 114 UCA: The unmatured, inferred germline sequence of mAb 114 with weak binding to ebolavirus GP [22].
S309: A patient-derived antibody that cross-neutralizes the sarbecoviruses SARS-CoV-1 (severe acute respiratory syndrome coronavirus 1) and SARS-CoV-2 by binding to the spike glycoprotein (Spike) [25] and is the parent antibody of sotrovimab [28], which currently has FDA emergency-use authorization (EUA) for treatment of COVID-19 (coronavirus disease 2019).
REGN10987: A patient-derived antibody that binds early variants of SARS-CoV-2 Spike and that had an FDA EUA for use against these variants [24].
C143: A patient-derived antibody that binds the SARS-CoV-2 Wuhan-Hu-1 Spike but was isolated prior to extensive in-vivo somatic hypermutation [29].
LY-1404 (Bebtelovimab): a patient-derived monoclonal antibody that was expected to neutralize multiple variants of SARS-CoV-2, but deauthorized for use in the U.S. due to lack of susceptibility to the drug by the BQ.1.1 variant [56], [57].
SA58: a patient-derived monoclonal antibody with broad-spectrum SARS-CoV-2 neutralizing activity [58], [59].

Table 5 shows a summary of information on the antibodies considered in each of the directed evolution campaigns. ‘Matured’ indicates extensive somatic hypermutation from germline (and, in the case of MEDI8852, additional in-vitro affinity maturation). ‘Source’ indicates how the antibody sequence was obtained; germline-inferred sequences were obtained from the original publications. ‘Improved binding’ is defined as a 1.1-fold improvement or higher from wildtype. ‘Preserved binding’ is defined as a sub-micromolar K_{d} for the screened antigen.

TABLE 5
Summary of antibodies considered in the study described herein.

| | | | | | Round-1 variants | | Best | Best |
|---|---|---|---|---|---|---|---|---|
| | | | | | with improved | Round-1 variants with | affinity | neutralization |
| | | | | Screened | binding or virus | preserved binding or | improvement | improvement |
| Antibody | Matured? | Source | Virus | antigen | neutralization | virus netutralization | (antigen) | (virus) |
| MEDI8852 | Yes (in | Patient- | Influenza | HA H4 | 2/13 = 15% | 13/14 = 93% | 7-fold | ND |
| | vivo + in | derived + | A | Hubei | | | (HA H7 | |
| | vitro) | in vitro | | | | | HK17) | |
| | | evolution | | | | | | |
| MEDI8852 | No | Germline- | Influenza | HA H1 | 3/8 = 38% | 8/8 = 100% | 23-fold (HA | ND |
| UCA | | inferred | A | Solomon | | | H4 Hubei) | |
| mAb114 | Yes | Patient- | Ebola- | GP | 6/13 = 46% | 13/13 = 100% | 3-fold (GP) | 1.5-fold (GP |
| | | derived | virus | | | | | pseudotyped |
| | | | | | | | | lentivirus) |
| mAb114 | No | Germline- | Ebola- | GP | 5/9 = 56% | 7/9 = 78% | 160-fold | >100-fold (GP |
| UCA | | inferred | virus | | | | (GP) | pseudotyped |
| | | | | | | | | lentivirus) |
| S309 | Yes | Patient- | Sarbeco- | Wuhan-Hu-1 | 2/10 = 20% | 9/10 = 90% | 1.7-fold | ND |
| | | derived | virus | S-6P | | | (Beta S-2P) | |
| REGN10987 | Yes | Patient- | SARS- | Beta S-2P | 2/8 = 25% | 8/8 = 100% | 1.3-fold | 2-fold (Beta |
| | | derived | CoV-2 | | | | (Beta S-2P) | pseudotyped |
| | | | | | | | | lentivirus) |
| C143 | No | Patient- | SARS- | Beta S-2P | 9/14 = 64% | 9/14 = 64% | 13-fold | 30-fold (Beta |
| | | derived | CoV-2 | | | | (Beta S-2P) | pseudotyped |
| | | | | | | | | lentivirus) |
| LY-1404 | Yes | Patient- | SARS- | BQ.1.1 | 13/20 = 65% | 20/20 = 100% (antigen) | 15-fold | 26-fold (BQ.1.1 |
| | | derived | CoV-2 | Spike RBD | (antigen) | 15/20 = 75% (BQ.1.1. | (BQ.1.1 | pseduotyped |
| | | | | | 9/20 = 45% | pseudotyped lentilvirus) | Spike RBD) | lentivirus) |
| | | | | | (BQ.1.1. pseudotyped | 20/20 = 100% (Wuhan. | | |
| | | | | | lentilvirus) | pseudotyped lentilvirus) | | |
| | | | | | 13/20 = 65% | | | |
| | | | | | (Wuhan. pseudotyped | | | |
| | | | | | lentilvirus) | | | |
| SA58 | Yes | Patient- | SARS- | BQ.1.1 | 7/20 = 35% | 19/20 = 95% (antigen) | 17-fold | 11-fold (BQ.1.1 |
| | | derived | CoV-2 | Spike | (antigen) | 17/20 = 85% (BQ.1.1. | (XBB.1.5 | pseduotyped |
| | | | | RBD | 3/20 = 15% | pseudotyped lentilvirus) | Spike RBD) | lentivirus) |
| | | | | XBB.1.5 | (BQ.1.1. pseudotyped | 13/20 = 65% (BA.1. | | |
| | | | | Spike RBD | lentilvirus) | pseudotyped lentilvirus) | | |
| | | | | | 5/20 = 25% | | | |
| | | | | | (BA.1. pseudotyped | | | |
| | | | | | lentilvirus) | | | |

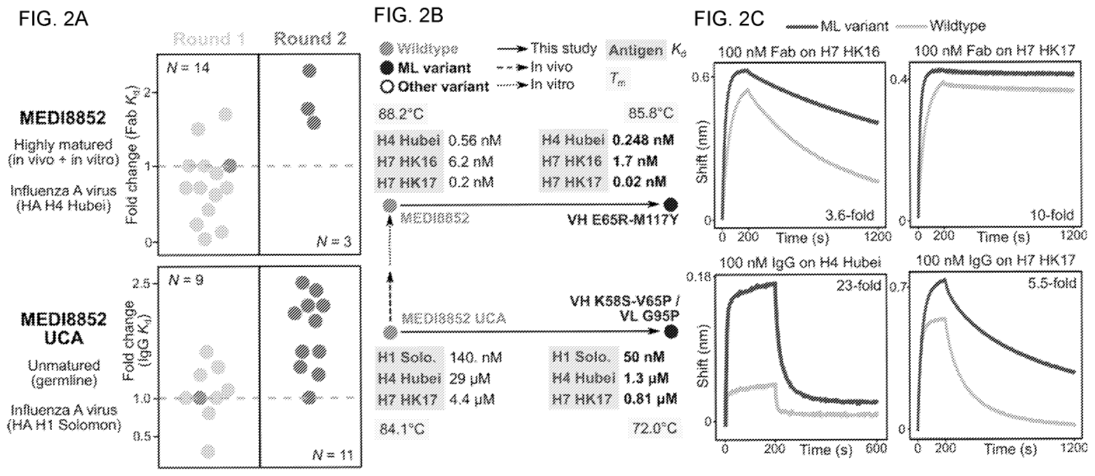

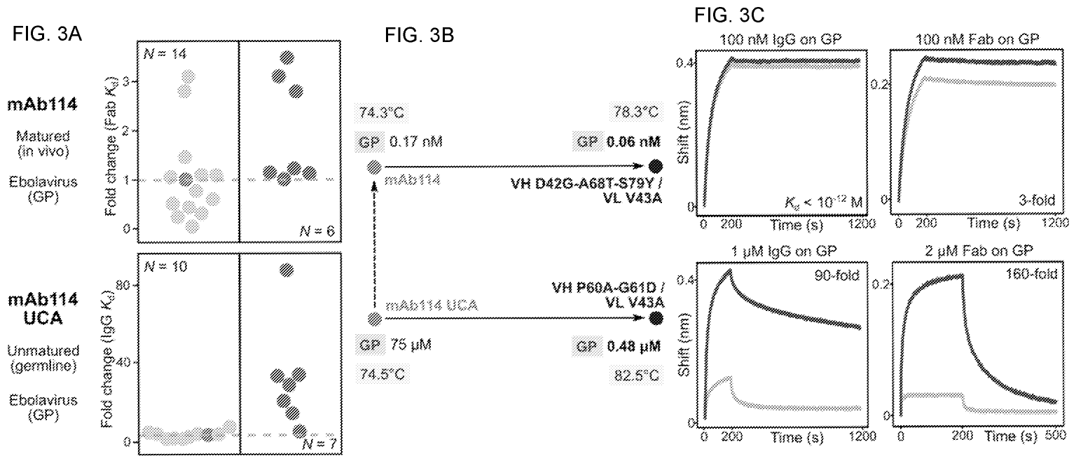

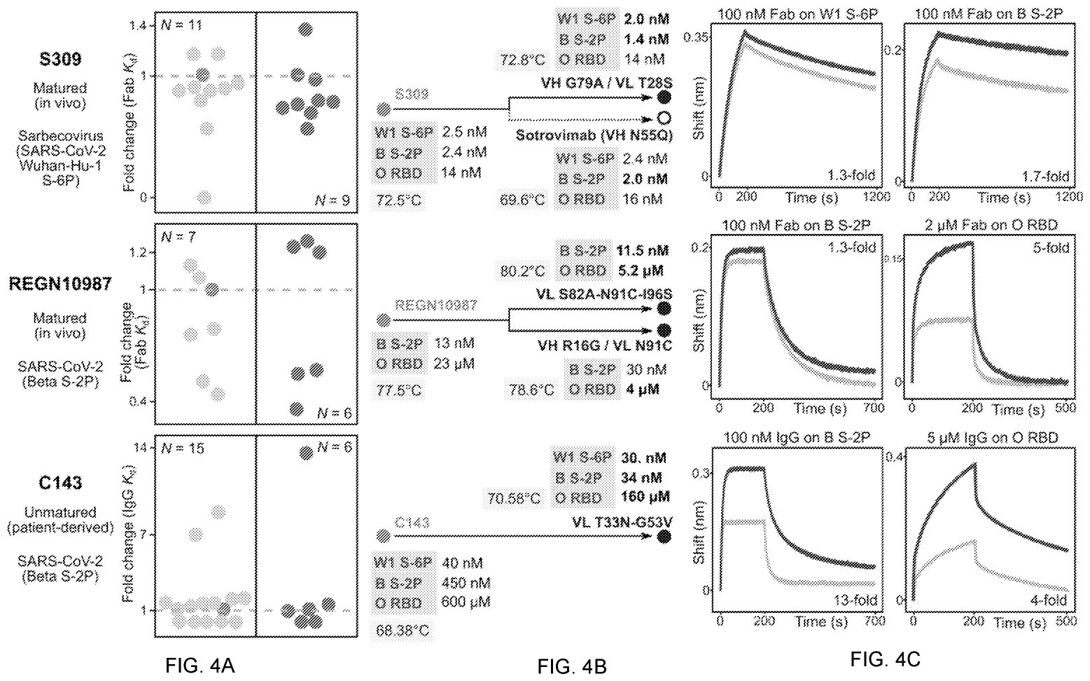

Evolution was performed by first measuring the highest likelihood mutations recommended by a consensus of the ESM-1b and ESM-1v ensemble of language models (six language models in total) [14], [15]. These language models were used to compute likelihoods of all single-residue mutations to the variable regions of the heavy chain (VH) or the light chain (VL). Based on these mutational likelihoods, mutations were acquired with higher evolutionary likelihood than wildtype according to two or more language models; additional details are provided in Example 1, above. In the first round of evolution, variants were measured that have single-residue substitutions from wildtype. In the second round, mutations with preserved or improved binding based on the results of the first round were combined. These two rounds were performed for all nine antibodies, and 8 to 14 variants per antibody were measured in round one and 1 to 11 variants per antibody were measured in round two (FIGS. 2A-4C, Table 5). Variants of the clinically-relevant antibodies, which have very low or undetectable dissociation as IgGs, were screened by measuring the dissociation constant (K_{d}) of the monovalent fragment antigen-binding (Fab) region; variants of the unmatured antibodies were screened by measuring the K_{d} of the bivalent IgG, followed up by also measuring the K_{d}s of the Fab fragments of the highest-avidity variants (see Example 1).

Successful expression was observed for all but one of 122 new variants across the seven evolutionary trajectories (a success rate of >99%). Notably, across all seven antibodies, it was found that 64% to 100% of the first-round, single-residue variants retained sub-micromolar binding to the antigen, and 15% to 65% percent of first-round variants led to improved binding affinity (defined as a 1.1-fold or higher improvement in K_{d} compared to wildtype) (Table 5). In combination, most of the first-round mutations also lead to improved binding, with some combinations demonstrating additive or synergistic mutational effects.

### Experimental Testing of Antibody Variants

### 1. MEDI8852 Antibody Variants

Table 6 shows the MEDI8852 variants tested. Variants were tested across two rounds of directed evolution. Table 6 shows the Kabat-annotated regions comprising the mutation(s) and K_{d}s for three HA antigens. In Round 2, VH E65R was added alone and in combination with M117Y as a result of a small-scale supervised learning experiment (see Example 1). The wildtype row is shown in bold text; variants with improved affinity are shown with italicized text. Table 7 shows binding affinity between MEDI8852 wildtype and three variants against a panel of nine Has. A K_{d} of <0.001 indicates an interaction with no observed dissociation when measured via BLI.

TABLE 6
MEDI8825 variants (NB: no binding; ND: not determined).

| | | | H4 Hubei | H7 HK16 | H7 HK17 |
|---|---|---|---|---|---|
| Round | Design | Region | Fab K_{d} (nM) | Fab K_{d} (nM) | Fab K_{d} (nM) |
| | MEDI8825 | | 0.56 ± 0.04 | 6.2 ± 0.8 | 0.2 ± 0.1 |
| | WT | | | | |
| Round 1 | VH 124V | HFR1 | 0.87 ± 0.01 | 8.58 ± 0.08 | ND |
| | VH D27F | HFR1 | 0.90 ± 0.07 | 9.0 ± 0.1 | ND |
| | VH S44G | HFR2 | 0.596 ± 0.005 | 7.03 ± 0.04 | ND |
| | VH T53I | CDR-H2 | 0.747 ± 0.009 | 10.26 ± 0.09 | ND |
| | VH E65P | CDR-H2 | 0.310 ± 0.005 | 2.33 ± 0.01 | 0.10 ± 0.02 |
| | VH N74S | HFR3 | 0.531 ± 0.005 | 5.98 ± 0.04 | ND |
| | VH P75R | HFR3 | 0.779 ± 0.009 | 7.14 ± 0.05 | ND |
| | VH M117Y | CDR-H3 | 0.356 ± 0.004 | 4.07 ± 0.03 | ND |
| | VL T25A | CDR-L1 | 1.45 ± 0.06 | 16.8 ± 0.4 | ND |
| | VL L29V | CDR-L1 | 4.2 ± 0.3 | 80 ± 10 | ND |
| | VL T33L | CDR-L1 | 3.2 ± 0.2 | 41 ± 3 | ND |
| | VL G55A | CDR-L2 | 0.512 ± 0.007 | 5.11 ± 0.04 | ND |
| | VL R92D | CDR-L3 | NB | 26.4 ± 0.5 | ND |
| | VL G95P | LFR4 | 0.802 ± 0.001 | 5.31 ± 0.04 | ND |
| Round 2 | VH E65R | CDR-H2 | 0.32 ± 0.02 | 1.54 ± 0.06 | 0.036 ± 0.004 |
| | VH E65P- | Multi | 0.36 ± 0.02 | 2.7 ± 0.2 | 0.043 ± 0.003 |
| | M117Y | | | | |
| | VH E65R- | Multi | 0.248 ± 0.001 | 1.7 ± 0.3 | 0.02 ± 0.01 |
| | M117Y | | | | |

TABLE 7
MEDI8852 binding to Group 1 and Group 2 HAs.

| Antigen | | K_{d} | K_{d} |
|---|---|---|---|
| (Group) | Antibody | (nM) | error (nM) |
| H1 Caledonia(Group 1) | MEDI8852 | <0.001 | Undefined |
| | VH E65P | <0.001 | Undefined |
| | VH E65R | <0.001 | Undefined |
| | VH E65R-M117Y | <0.001 | Undefined |
| H1 Solomon | MEDI8852 | <0.001 | Undefined |
| (Group 1) | VH E65P | <0.001 | Undefined |
| | VH E65R | <0.001 | Undefined |
| | VH E65R-M117Y | <0.001 | Undefined |
| H2 Japan | MEDI8852 | <0.001 | Undefined |
| (Group 1) | VH E65P | <0.001 | Undefined |
| | VH E65R | <0.001 | Undefined |
| | VH E65R-M117Y | <0.001 | Undefined |
| H3 Panama | MEDI8852 | <0.001 | Undefined |
| (Group 2) | VH E65P | <0.001 | Undefined |
| | VH E65R | <0.001 | Undefined |
| | VH E65R-M117Y | <0.001 | Undefined |
| H3 Victoria | MEDI8852 | <0.001 | Undefined |
| (Group 2) | VH E65P | <0.001 | Undefined |
| | VH E65R | <0.001 | Undefined |
| | VH E65R-M117Y | <0.001 | Undefined |
| H4 Hubei | MEDI8852 | 0.56 | 0.04 |
| (Group 2) | VH E65P | 0.310 | 0.005 |
| | VH E65R | 0.32 | 0.02 |
| | VH E65R-M117Y | 0.248 | 0.001 |
| H5 Vietnam | MEDI8852 | <0.001 | Undefined |
| (Group 1) | VH E65P | <0.001 | Undefined |
| | VH E65R | <0.001 | Undefined |
| | VH E65R-M117Y | <0.001 | Undefined |
| H7 HK16 | MEDI8852 | 6.2 | 0.8 |
| (Group 2) | VH E65P | 2.33 | 0.01 |
| | VH E65R | 1.54 | 0.06 |
| | VH E65R-M117Y | 1.7 | 0.3 |
| H7 HK17 | MEDI8852 | 0.2 | 0.1 |
| (Group 2) | VH E65P | 0.10 | 0.02 |
| | VH E65R | 0.036 | 0.004 |
| | VH E65R-M117Y | 0.02 | 0.01 |

### 2. MED8852 UCA Variants

Table 8 shows the MED18852 UCA variants tested. Variants were tested across two rounds of directed evolution. Table 8 shows the Kabat-annotated regions comprising the mutation(s) and K_{d}s for three HA antigens. In Round 2, all possible combinations involving K58S, V65P, P75R in the VH and G95P in the VL were made. The wildtype row is shown in bold text; variants with improved affinity are shown with italicized text.

TABLE 8
MEDI8852 UCA variants (ND: not determined).

| | | | H1 | H1 | | | |
|---|---|---|---|---|---|---|---|
| | | | Solomon | Solomon | H4 Hubei | H4 Hubei | |
| | | | IgG K_{d} | Fab K_{d} | IgG K_{d} | Fab K_{d} | H7 HK17 |
| Round | Design | Region | (nM) | (nM) | (nM) | (μM) | K_{d} (nM) |
| | MEDI8852 UCA | | 2.0 ± 0.2 | 140. ± 2 | 740 ± 30 | 29 ± 3 | IgG K_{d}: |
| | WT | | | | | | 17 ± 4 |
| | | | | | | | Fab K_{d}: |
| | | | | | | | 4400 ± 500 |
| Round 1 | VH S44G | HFR2 | 2.1 ± 0.4 | ND | ND | ND | ND |
| | VH T53I | CDR-H2 | 2.1 ± 0.4 | ND | ND | ND | ND |
| | VH K58S | CDR-H2 | 1.5 ± 0.4 | 110 ± 10 | ND | 2.5 ± 0.7 | ND |
| | VH V65P | CDR-H2 | 1.7 ± 0.4 | ND | ND | ND | ND |
| | VH N74S | HFR3 | 2.6 ± 0.3 | ND | ND | ND | ND |
| | VH P75R | HFR3 | 2.0 ± 0.2 | ND | ND | ND | ND |
| | VL N34A | CDR-L1 | 8.2 ± 0.5 | ND | ND | ND | ND |
| | VL G95P | LFR4 | 1.3 ± 0.2 | 64.9 ± 0.9 | ND | 17 ± 9 | ND |
| Round 2 | VH K58S-V65P | CDR-H2 | 1.3 ± 0.1 | ND | 80 ± 20 | ND | ND |
| | VH K58S-P75R | Multi | 1.2 ± 0.2 | ND | 47 ± 9 | ND | ND |
| | VH V65P-P75R | Multi | 1.53 ± 0.09 | ND | 570 ± 10 | ND | ND |
| | VH K58S-V65P- | Multi | 1.22 ± 0.04 | ND | 72.0 ± 0.3 | ND | ND |
| | P75R | | | | | | |
| | VH K58S/VL | Multi | 0.96 ± 0.02 | 56 ± 7 | 36 ± 2 | 3 ± 2 | ND |
| | G95P | | | | | | |
| | VH V65P/VL | Multi | 0.93 ± 0.05 | ND | 100 ± 20 | ND | ND |
| | G95P | | | | | | |
| | VH P75R/VL | Multi | 0.95 ± 0.02 | ND | 107 ± 6 | ND | ND |
| | G95P | | | | | | |
| | VH K58S- | Multi | 0.78 ± 0.08 | 50 ± 10 | 30. ± 1 | 1.3 ± 0.2 | IgG K_{d}: |
| | V65P/VL G95P | | | | | | 5.6 ± 0.9 |
| | | | | | | | Fab K_{d}: |
| | | | | | | | 810 ± 10 |
| | VH K58S- | Multi | 0.88 ± 0.05 | ND | 45 ± 2 | ND | ND |
| | P75R/VL G95P | | | | | | |
| | VH V65P- | Multi | 0.87 ± 0.04 | ND | 110 ± 20 | ND | ND |
| | P75R/VL G95P | | | | | | |
| | VH K58S-V65P- | Multi | 0.79 ± 0.08 | 55 ± 4 | 42 ± 5 | 1.5 ± 0.4 | ND |
| | P75R/VL G95P | | | | | | |

### 3. mAb114 Antibody Variants

Table 9 shows the mAb114 variants tested. Variants were tested across two rounds of directed evolution. Table 9 shows the Kabat-annotated regions comprising the mutation(s) and K_{d}s for ebolavirus GP. Neutralization IC₅₀S were also determined for mAb114 WT and all Round-2 variant IgGs against GP-pseudotyped lentivirus. The wildtype row is shown in bold text; variants with improved affinity are shown with italicized text.

TABLE 9
mAb114 variants (ND: not determined).

| | | | | Ebola GP |
|---|---|---|---|---|
| | | | GP | pseudotyped |
| | | | Fab K_{d} | lentivirus |
| Round | Design | Region | (nM) | IC₅₀ (nM) |
| | mAb114 WT | | 0.17 ± 0.05 | 1.43 ± 0.09 |
| Round | VH M31S | CDR-H1 | 0.1 ± 0.2 | ND |
| 1 | VH I41P | HFR2 | 0.3 ± 0.4 | ND |
| | VH D42G | HFR2 | 0.2 ± 0.1 | ND |
| | VH A68T | HFR3 | 0.1 ± 0.2 | ND |
| | VH E72D | HFR3 | 0.1 ± 0.2 | ND |
| | VH S79Y | HFR3 | 0.1 ± 0.3 | ND |
| | VH I113T | HFR4 | 0.07 ± 0.06 | ND |
| | VL I19V | LFR1 | 0.2 ± 0.2 | ND |
| | VL F29I | CDR-L1 | 0.5 ± 0.4 | ND |
| | VL V43A | LFR2 | 0.07 ± 0.07 | ND |
| | VL S49Y | LFR2 | 0.6 ± 0.2 | ND |
| | VL H70D | LFR3 | 0.3 ± 0.1 | ND |
| | VL N90Q | CDR-L3 | 4.0 ± 0.3 | ND |
| Round | VH D42G-A68T- | Multi | 0.19 ± 0.02 | 1.2 ± 0.2 |
| | S79Y | | | |
| 2 | VH I41P-D42G- | Multi | 0.19 ± 0.01 | 1.2 ± 0.2 |
| | A68T-S79Y-I113T | | | |
| | VH A68T-I113T / | Multi | 0.17 ± 0.01 | 1.1 ± 0.4 |
| | VL V43A | | | |
| | VH A68T-E72D- | Multi | 0.076 ± 0.005 | 1.2 ± 0.3 |
| | S79Y-I113T / | | | |
| | VL V43A | | | |
| | VH D42G-A68T- | Multi | 0.06 ± 0.01 | 0.96 ± 0.07 |
| | S79Y / VL | | | |
| | V43A | | | |
| | VH I41P-D42G- | Multi | 0.069 ± 0.002 | 1.0 ± 0.2 |
| | A68T-S79Y- | | | |
| | I113T / VL V43A | | | |

### 4. mAb114 UCA Variants

Table 10 shows the mAb 114 UCA variants tested. Variants were tested across two rounds of directed evolution. Table 10 shows the Kabat-annotated regions comprising the mutation(s) and K_{d}s (for both IgG and Fab versions) for ebolavirus GP. The wildtype row is shown in bold text; variants with improved affinity are shown with italicized text.

TABLE 10
mAb114 UCA variants (ND: not determined).

| Round | Design | Region | GP IgG K_{d} (nM) | GP Fab K_{d} (μM) |
|---|---|---|---|---|
| | mAb114 UCA WT | | 400 ± 70 | 75 ± 3 |
| Round 1 | VH T41P | HFR2 | 100 ± 100 | ND |
| | VH A54G | CDR-H2 | 80000 ± 60000 | ND |
| | VH P60A | CDR-H2 | 40000 ± 20000 | ND |
| | VH G61D | CDR-H2 | 100 ± 200 | ND |
| | VH E72D | HFR3 | 200 ± 200 | ND |
| | VH G88E | HFR3 | 150 ± 20 | ND |
| | VH V96A | HFR3 | 110 ± 30 | ND |
| | VL V43A | LFR2 | 60 ± 10 | ND |
| | VL K90Q | CDR-L3 | 620 ± 60 | ND |
| Round 2 | VH P60A-G61D | CDR-H2 | 120 ± 20 | ND |
| | VH T41P-P60A-G61D- | Multi | 33 ± 8 | ND |
| | E72D-G88E-V96A | | | |
| | VH G88E / VL V43A | Multi | 13 ± 3 | ND |
| | VH V96A / VL V43A | Multi | 22 ± 7 | ND |
| | VH P60A-G61D / | Multi | 5 ± 4 | 0.48 ± 0.01 |
| | VL V43A | | | |
| | VH P60A-G61D-G88E- | Multi | 13 ± 3 | ND |
| | V96A / | | | |
| | VL V43A | | | |
| | VH T41P-P60A-G61D- | Multi | 15 ± 2 | ND |
| | E72D-G88E-V96A / | | | |
| | VL V43A | | | |

### 5. S309 Antibody Variants

Table 11 shows the S309 variants tested. Variants were tested across two rounds of directed evolution. Table 11 shows the Kabat-annotated regions comprising the mutation(s) and K_{d}s for antigens from three SARS-CoV-2 variants. The wildtype row is shown in bold text; variants with improved affinity are shown with italicized text.

TABLE 11
S309 variants (W1: Wuhan-Hu-1; ND: not determined)

| | | | W1 | Beta | Omicron |
|---|---|---|---|---|---|
| | | | S-6P Fab | S-2P Fab | RBD Fab |
| Round | Design | Region | K_{d} (nM) | K_{d} (nM) | K_{d} (nM) |
| | S309 WT | | 2.5 ± 0.2 | 2.4 ± 0.2 | 14 ± 1 |
| | Sotrovimab | CDR-H2 | 2.4 ± 0.3 | 2.0 ± 0.4 | 16 ± 3 |
| | (VH N55Q) | | | | |
| Round 1 | VH P28T | HFR1 | 4 ± 1 | ND | ND |
| | VH T77N | HFR3 | 2.8 ± 0.5 | ND | ND |
| | VH G79A | HFR3 | 2.8 ± 0.5 | ND | ND |
| | VH R84S | HFR3 | 2.7 ± 0.3 | ND | ND |
| | VH R85S | HFR3 | 3.1 ± 0.7 | ND | ND |
| | VH R87T | HFR3 | 2.1 ± 0.3 | ND | ND |
| | VL T28S | CDR-L1 | 2.1 ± 0.3 | ND | ND |
| | VL T32S | CDR-L1 | 2.7 ± 0.3 | ND | ND |
| | VL S95V | CDR-L3 | NE | ND | ND |
| | VL L96P | CDR-L3 | 2.67 ± 0.05 | ND | ND |
| Round 2 | VH T77N-G79A-R84S | HFR3 | 3.2 ± 0.6 | ND | ND |
| | VH T77N-G79A-R84S- | HFR3 | 3.1 ± 0.6 | ND | ND |
| | R85S | | | | |
| | VL T28S-T32S | CDR-L1 | 3 ± 1 | ND | ND |
| | VH G79A / | Multi | 2.0 ± 0.3 | 1.4 ± 0.2 | 14 ± 3 |
| | VL T28S | | | | |
| | VH R84S / VL T28S | Multi | 2.5 ± 0.8 | ND | ND |
| | VH R87T / VL T28S | Multi | 2.5 ± 0.7 | ND | ND |
| | VH T77N-G79A-R84S / | Multi | 3 ± 1 | ND | ND |
| | VL T28S | | | | |
| | VH T77N-G79A-R84S / | Multi | 3.5 ± 0.7 | ND | ND |
| | VL T28S-T32S | | | | |
| | VH T77N-G79A-R84S- | Multi | 4 ± 1 | ND | ND |
| | R85S / | | | | |
| | VL T28S-T32S | | | | |

### 6. REGN10987 Antibody Variants

Table 12 shows REGN10987 variants tested. Variants were tested across two rounds of directed evolution. Table 12 shows the Kabat-annotated regions comprising the mutation(s) and K_{d}s for antigens from two SARS-CoV-2 variants. Round-1 variants were pre-screened as IgGs with a single replicate before testing the highest-avidity variants as Fabs and with multiple replicates. Neutralization IC₅₀s were also determined for REGN10987 WT and selected affinity-enhancing variant IgGs against D614G- and Beta-pseudotyped lentivirus. The wildtype row is shown in bold text; variants with improved affinity are shown with italicized text.

TABLE 12
REGN10987 variants (ND: not determined).

| | | | Beta | | | | Beta- |
|---|---|---|---|---|---|---|---|
| | | | S-2P | | | D614G- | pseudotyped |
| | | | IgG | Beta S-2P | Omicron | pseudotyped | lentivirus |
| | | | K_{d} | Fab K_{d} | RBD K_{d} | lentivirus | IC₅₀ |
| Round | Design | Region | (nM) | (nM) | (μM) | IC₅₀ (μg/mL) | (μg/mL) |
| | REGN10987 WT | | 0.09 | 13 ± 3 | IgG K_{d}: | 1.1 ± 0.4 | 1.4 ± 0.2 |
| | | | | | 0.7 ± 0.1 | | |
| | | | | | Fab K_{d}: | | |
| | | | | | 23 ± 5 | | |
| Round 1 | VH R16G | HFR1 | 0.07 | 10. ± 4 | 0.51 ± 0.06 | 0.8 ± 0.2 | 0.7 ± 0.1 |
| | VH S98R | HFR3 | 0.41 | ND | ND | ND | ND |
| | VH V108D | CDR-H3 | 0.08 | 26 ± 5 | ND | ND | ND |
| | VL S82A | LFR3 | 0.06 | 14 ± 3 | ND | ND | ND |
| | VL N91C | CDR-L3 | 0.05 | 11 ± 2 | ND | ND | ND |
| | VL N91S | CDR-L3 | 0.07 | 22 ± 8 | ND | ND | ND |
| | VL L93Y | CDR-L3 | 0.30 | ND | ND | ND | ND |
| | VL I96S | CDR-L3 | 0.07 | 15 ± 2 | ND | ND | ND |
| Round 2 | VH R16G-V108D | Multi | ND | 30 ± 10 | 2.2 ± 0.4 | ND | ND |
| | VL S82A-N91C-I96S | Multi | ND | 11.5 ± 0.8 | 0.48 ± 0.01 | 1.04 ± 0.3 | 0.87 ± 0.09 |
| | VH R16G/VL S82A | Multi | ND | 12 ± 4 | 0.9 ± 0.4 | 1.04 ± 0.3 | 0.8 ± 0.2 |
| | VH R16G/VL N91C | Multi | ND | 30 ± 10 | IgG K_{d}: | ND | ND |
| | | | | | 0.417 ± | | |
| | | | | | 0.002 | | |
| | | | | | Fab K_{d}: | | |
| | | | | | 4 ± 1 | | |
| | VH R16G/VL I96S | Multi | ND | 12 ± 4 | 1.2 ± 0.3 | 1.2 ± 0.4 | 0.8 ± 0.1 |
| | VH R16G-V108D/VL | Multi | ND | 40 ± 30 | 2.5 ± 0.7 | ND | ND |
| | S82A-N91C-I96S | | | | | | |

### 7. C143 Antibody Variants

Table 13 shows the C143 variants tested. Variants were tested across two rounds of directed evolution. Table 13 shows the Kabat-annotated regions comprising the mutation(s) and K_{d}s for antigens from three SARS-CoV-2 variants. Neutralization IC₅₀s were also determined for C143 WT and selected affinity-enhancing variant IgGs against D614G- and Beta-pseudotyped lentivirus. The wildtype row is shown in bold text; variants with improved affinity are shown with italicized text.

TABLE 13
C143 variants (W1: Wuhan-Hu-1; NB: no binding; ND: not determined.

| | | | | | | D614G | Beta |
|---|---|---|---|---|---|---|---|
| | | | Beta S-2P | W1 S-6P | Omicron | pseudotyped | pseudotyped |
| | | | IgG K_{d} | IgG K_{d} | IgG K_{d} | lentivirus | lentivirus |
| Round | Design | Region | (nM) | (nM) | (μM) | IC₅₀ (μg/mL) | IC₅₀ (μg/mL) |
| | C143 WT | | 450 ± 40 | 40 ± 10 | 600 ± 100 | 1400 ± 300 | 6000 ± 2000 |
| Round 1 | VH V29F | HFR1 | 140 ± 10 | ND | ND | ND | ND |
| | VH K32Y | CDR-H1 | 30000 ± 40000 | ND | ND | ND | ND |
| | VH L51Y | CDR-H2 | 200 ± 100 | ND | ND | ND | ND |
| | VH D57T | CDR-H2 | NB | ND | ND | ND | ND |
| | VH A77T | HFR3 | 200 ± 100 | ND | ND | ND | ND |
| | VH G91A | HFR3 | 200 ± 100 | ND | ND | ND | ND |
| | VL N27S | CDR-L1 | 100 ± 100 | ND | ND | ND | ND |
| | VL T33N | CDR-L1 | 40 ± 20 | ND | ND | 200 ± 60 | 900 ± 400 |
| | VL L34Y | CDR-L1 | 200 ± 200 | ND | ND | ND | ND |
| | VL Y41H | LFR2 | 110 ± 10 | ND | ND | ND | ND |
| | VL G53V | CDR-L2 | 30 ± 10 | ND | ND | 110 ± 10 | 180 ± 60 |
| | VL S57P | CDR-L2 | NB | ND | ND | ND | ND |
| | VL G82A | LFR3 | NB | ND | ND | ND | ND |
| | VL A96S | CDR-L3 | 300 ± 200 | ND | ND | ND | ND |
| Round 2 | VH L51Y-A77T- | Multi | NB | ND | ND | ND | ND |
| | G91A | | | | | | |
| | VL T33N-G53V | Multi | 34 ± 9 | 30. ± 8 | 160 ± 50 | 80 ± 20 | 300 ± 100 |
| | VL N27S-T33N- | Multi | 520 ± 30 | ND | ND | ND | ND |
| | L34Y-G53V | | | | | | |
| | VL N27S-T33N- | Multi | 800 ± 100 | ND | ND | ND | ND |
| | L34Y-Y41H- | | | | | | |
| | G53V-S57P- | | | | | | |
| | G82A | | | | | | |
| | VH L51Y-A77T- | Multi | NB | ND | ND | ND | ND |
| | G91A/VL T33N | | | | | | |
| | VH L51Y-A77T- | Multi | 300 ± 200 | ND | ND | ND | ND |
| | G91A/VL N27S- | | | | | | |
| | T33N-L34Y- | | | | | | |
| | G53V | | | | | | |

TABLE 14
LY-1404 variants (NA: non-applicable; ND: not determined; LOQ: limit of quantification).

| | | | Wuhan pseudotyped | | | BQ.1.1 pseudotyped | |
|---|---|---|---|---|---|---|---|
| | | | lentivirus IC₅₀ | | | lentivirus IC₅₀ | |
| | | | | Fold | BQ.1.1 RBD | | Fold |
| Round | Design | Region | μg/mL | change | IgG K_{d} (μM) | μg/mL | change |
| | WT | WT | 3.1 ± 1.1 | NA | 1.7 ± 0.01 | 110 ± 32 | NA |
| Round | D88Q | HFR3 | 1.4 | 2.2 | 1.06 ± 1.2 | 58 ± 19 | 1.9 |
| 1 | V90S | HFR3 | 1.8 | 1.7 | 0.19 ± 0.01 | 33 ± 13 | 3.3 |
| | S62N | CDR-H2 | 1.6 | 1.9 | 0.19 ± 0.01 | 36 ± 20 | 3 |
| | V81T | HFR3 | 2.2 | 1.4 | 0.61 ± 0.6 | 66 ± 15 | 1.7 |
| | F24Y | HFR1 | 3.3 | 0.9 | 0.44 ± 0.4 | 33 ± 6 | 3.3 |
| | I31T | CDR-H1 | 2.4 | 1.3 | 0.21 ± 0.01 | 68 ± 22 | 1.6 |
| | H99Y | HFR3 | 5.1 | 0.6 | 0.87 ± 0.3 | 42 ± 10 | 2.6 |
| | T70S | HFR3 | 3.3 | 0.9 | 0.18 ± 0.01 | 44 ± 5 | 2.5 |
| | I105L | CDR-H3 | 1.4 | 2.2 | 0.35 ± 0.02 | >LOQ | ND |
| | A98I | CDR-L3 | 1.7 | 1.8 | 2.28 ± 2.9 | >LOQ | ND |
| | Q39K | LFR2 | 2.2 | 1.4 | 0.13 ± 0.06 | 93 ± 41 | 1.2 |
| | T5Q | LFR1 | 2.6 | 1.2 | 1.77 ± 0.4 | 110 ± 40 | 1 |
| | K47E | LFR2 | 2.2 | 1.4 | 1.31 ± 0.3 | 140 ± 46 | 0.7 |
| | F51Y | LFR2 | 2.6 | 1.2 | 1.95 ± 0.3 | 150 ± 23 | 0.7 |
| | K44E | LFR2 | 2.7 | 1.2 | 2.42 ± 0.2 | 160 ± 65 | 0.6 |
| | M49L | LFR2 | 2.1 | 1.4 | 2.62 ± 0.04 | >LOQ | ND |
| | Q6S | LFR1 | 1.9 | 1.6 | 3.57 ± 0.5 | >LOQ | ND |
| Round | V90S/E85A | HFR3/LFR3 | 1.5 | 2 | 0.23 ± 0.005 | 11 ± 1.2 | 10 |
| 2 | S62N/E85A | CDR-H2/LFR3 | 1.3 | 2.3 | 0.22 ± 0.0004 | 8.7 ± 0.74 | 12 |
| | T70S/E85A | HFR3/LFR3 | 1.3 | 2.3 | 0.18 ± 0.002 | 11 ± 1.5 | 10 |
| | V90S/Q39K | HFR3/LFR2 | 0.89 | 3.5 | 0.18 ± 0.001 | 18 ± 0.45 | 6.1 |
| | S62N/Q39K | CDR-H2/LFR2 | 1.1 | 2.8 | 0.18 ± 0.003 | 15 ± 0.44 | 7.3 |
| | T70S/Q39K | HFR3/LFR2 | 1.7 | 1.8 | 0.16 ± 0.002 | 26 ± 0.65 | 4.2 |
| Round | D88Q_V90S_S62N_V81T | VH | 5.1 ± 1.1 | 0.6 | ND | 11 ± 3.6 | 10.1 |
| 3 | F24Y_I31T_H99Y_T70S | | | | | | |
| | D88Q_V90S_S62N_V81T | VH | 5.3 ± 2.5 | 0.5 | ND | 18 ± 1.2 | 6.2 |
| | F24Y_I31T_H99Y | | | | | | |
| | D88Q_V90S_S62N_V81T | VH | 5.9 ± 0.057 | 0.5 | ND | 4.3 ± 1.3 | 26 |
| | F24Y_H99Y_T70S | | | | | | |
| | D88Q_V90S_S62N_V81T | VH | 3.3 ± 1.7 | 0.9 | ND | 44 ± 9.9 | 2.5 |
| | F24Y_I31T_T70S | | | | | | |
| | D88Q_V90S_S62N_V81T | VH | 2.3 ± 0.55 | 1.3 | ND | 12 ± 3.1 | 9.1 |
| | I31T_H99Y_T70S | | | | | | |

TABLE 15
SA58 variants (NA: non-applicable; ND: not determined).

| | | | | | | BA.1 pseudotyped | | BQ.1.1 pseudotyped | |
|---|---|---|---|---|---|---|---|---|---|
| | | | BQ.1.1 | XBB | XBB.1.5 | lentivirus IC₅₀ | | lentivirus IC₅₀ | |
| | | | RBD IgG | RBD IgG | RBD IgG | | Fold | | Fold |
| Round | Design | Region | K_{d} (pM) | K_{d} (μM) | K_{d} (μM) | μg/mL | change | μg/mL | change |
| | WT | | <1 | 7.4 ± 8.4 | 2.4 ± 0.3 | 22 ± 12 | NA | 6.4 ± 2.1 | NA |
| Round | T53L | CDR-H2 | <1 | 0.97 ± 0.006 | 0.98 ± 0.5 | 19 ± 4.6 | 1.1 | 4.5 ± 0.50 | 1.40 |
| 1 | A61S | CDR-H2 | <1 | 1.41 ± 0.4 | 2.9 ± 0.5 | 21 ± 6.2 | 1.0 | 5 ± 0.91 | 1.20 |
| | E10Q | HFR1 | <1 | 7.5 ± 7.8 | 3.0 ± 0.6 | 15 ± 3.5 | 1.0 | 7.2 ± 0.76 | 0.80 |
| | N95V | CDR-L3 | <1 | 0.37 ± 0.06 | 0.21 ± 0.003 | 9.1 ± 0.27 | 2.4 | 0.93 ± 0.33 | 6.80 |
| | S85A | LFR3 | <1 | 12 ± 15 | 2.02 ± 0.1 | 8.1 ± 6.8 | 2.7 | 6.8 ± 3.4 | 0.90 |
| | S54T | CDR-L2 | <1 | 2.4 ± 1 | 24 ± 31 | 12 ± 11 | 1.8 | 9.7 ± 0.87 | 0.60 |
| | M4V | LFR1 | <1 | 0.67 ± 0.4 | 0.33 ± 0.01 | 10. ± 9.9 | 2.2 | 10 ± 1.6 | 0.60 |
| Round | T53L/N95V | CDR-H2/CDR-L3 | <1 | 0.21 ± 0.009 | 0.14 ± 0.02 | 9.6 ± 1.1 | 2.3 | 0.56 ± 0.019 | 11.4 |
| 2 | Q82T/N95V | HFR3/CDR-L3 | <1 | 0.59 ± 0.07 | 0.49 ± 0.05 | 9.8 | 2.2 | 1.8 ± 0.72 | 3.5 |
| | A61S/N95V | CDR-H2/CDR-L3 | <1 | 0.57 ± 0.02 | 0.49 ± 0.1 | 10 | 2.2 | 0.95 ± 0.38 | 6.7 |
| | T53L/M4V | CDR-H2/LFR1 | <1 | 0.27 ± 0.03 | 0.17 ± 0.03 | 19 | 1.1 | 6 ± 1.4 | 1 |
| | Q82T/M4V | HFR3/LFR1 | <1 | 0.64 ± 0.3 | 0.47 ± 0.005 | 23 | 0.9 | 12 ± 0.63 | 0.5 |
| | A61S/M4V | CDR-H2/LFR1 | <1 | 0.62 | 0.56 ± 0.1 | 15 | 1.4 | 11 ± 0.16 | 0.5 |
| | T53L/A61S | CDR-H2_CDR- | ND | ND | ND | 12 ± 12 | 1.8 | 1.7 ± 0.4 | 3.8 |
| | | H2 | | | | | | | |

### 8. LY-1404 Antibody Variants

Table 14 shows the LY-1404 antibody variants tested. Variants were tested across three rounds of directed evolution. Table 14 shows the Kabat-annotated regions comprising the mutation(s) and K_{d}s for antigen for SARS-CoV-2 BQ.1.1 variant. Neutralization IC₅₀s were also determined for Wuhan- and BQ.1.1-pseudotyped lentivirus.

### 9. SA58 Antibody Variants

Table 15 shows the SA58 antibody variants tested. Variants were tested across two rounds of directed evolution. Table 15 shows the Kabat-annotated regions comprising the mutation(s) and K_{d}s for antigen for SARS-CoV-2 BQ.1.1, XBB, and XBB1.5 variants. Neutralization IC₅₀s were also determined for BA.1- and BQ.1.1-pseudotyped lentivirus.

### 10. Results

Binding affinities were improved for all clinically-relevant antibodies tested, despite these antibodies being highly evolved. MED18852 is a potent binder with a sub-picomolar Fab K_{d} across many HAs but with picomolar or nanomolar binding to HAs from subtypes H4 and H7. While variants were explicitly screened using an HA H4 antigen, the best design improved binding across a broad set of HAs (Table 7), including a 10-fold improvement for HA H7 HK17 (A/Hong Kong/125/2017(H7N9)). The best variant of mAb114, a clinically-approved drug, achieved a 3-fold improvement in K_{d} for ebolavirus GP. For REGN10987, the highest-affinity variant against Beta S-2P (the antigen used in screening) has a 1.3-fold improvement, while another of the designs has a 5-fold improvement for Omicron RBD. For S309, the designs were compared to wildtype and to a variant with the N55Q mutation in the VH introduced by Alexander et al. after a small-scale, rational evolutionary screen [28]; the S309 Fab with the VH N55Q mutation forms the Fab of the therapeutic antibody, sotrovimab. Interestingly, the best variant of S309 has higher affinity than sotrovimab, including a 1.3-fold improvement in K_{d} compared to wildtype S309 (versus 1.1-fold for sotrovimab) for the SARS-CoV-2 Wuhan-Hu-1 Spike with six stabilizing proline substitutions (S-6P) (the antigen used in screening), a 1.8-fold improvement (versus 1.3-fold for sotrovimab) for the Beta-variant Spike with two stabilizing proline substitutions (S-2P), a 0.94-fold change (versus 0.83-fold for sotrovimab) for the Omicron-variant receptor binding domain (RBD). These and other results are further summarized in FIGS. 2A-4C and Table 5-Table 15.

Affinities were also improved for all three unmatured antibodies, often involving much higher fold changes than when evolving the matured antibodies, indicating easier evolvability with respect to affinity. For MEDI8852 UCA, the best Fab design achieved a 3-fold improvement in K_{d} against HA H1 Solomon (A/Solomon Islands/3/2006(H1N1)), the antigen used in screening. The best designs also acquired breadth of binding to some Group 2 HAs, including a 23-fold improvement for HA H4 Hubei (A/swine/Hubei/06/2009(H4N1)) and a 5-fold improvement for HA H7 HK17. For mAb 114 UCA, the best Fab design achieved a 160-fold improvement in K_{d} for ebolavirus GP. While some of the model's recommended mutations to these UCA antibodies are also observed in the matured antibody, other affinity-enhancing mutations are not. Excluding any mutations or mutated sites found in the matured antibody, the UCA variants achieved a 7-fold improvement for HA H4 Hubei and a 33-fold improvement for ebolavirus GP, demonstrating that the algorithm successfully explores alternative evolutionary routes. For C143, a patient-derived antibody isolated prior to extensive affinity maturation, the best design achieved a 13-fold improvement for Beta S-2P and a 3.8-fold improvement for Omicron RBD. These and other results are further summarized in FIGS. 2A-4C and Table 5-Table 15. In total, across antibodies representing diverse antigens and degrees of maturity, the approach described herein consistently designed higher-affinity variants.

### Example 3. Improved Thermostability and Neutralization of Evolved Antibodies

Although variants were explicitly selected for improved binding to specific antigens, they were also assayed for improved stability. 21 out of the 31 high-likelihood variants that were tested have a higher Fab melting temperature (Tₘ) than wildtype, and all variants maintain thermostability. Interestingly, when evolving S309 to have higher affinity, the design described herein has a Tₘ of 72.8° C. compared to 72.5° C. for wildtype, whereas the VH N55Q mutation introduced in sotrovimab decreases the Tₘ to 69.6° C. (FIGS. 2A-4C). The evolved variants described herein for mAb 114, mAb 114 UCA, REGN10987, and C143 also preserve or improve Tₘ; the highest change observed is an increase from 74.5° C. to 82.5° C. when evolving mAb114 UCA. Improved thermostability does not completely explain the affinity maturation results, however, as somewhat decreased Tₘ was observed for the affinity-matured variants of MEDI8852 and its UCA, though these Fabs are still thermostable at high temperatures (FIGS. 2A-4C).

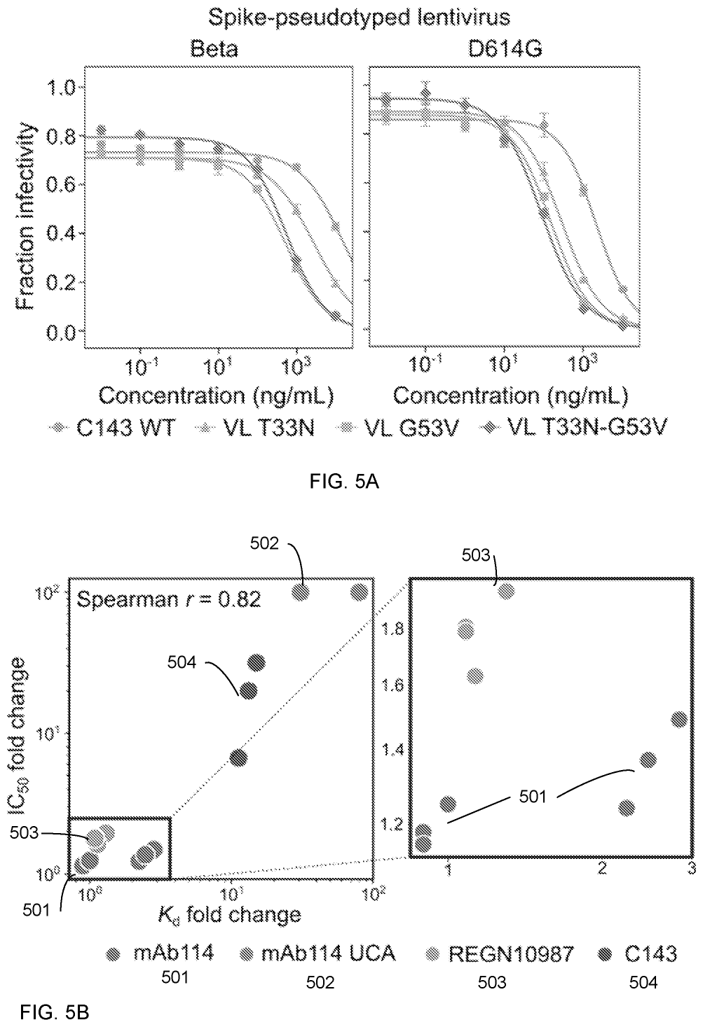

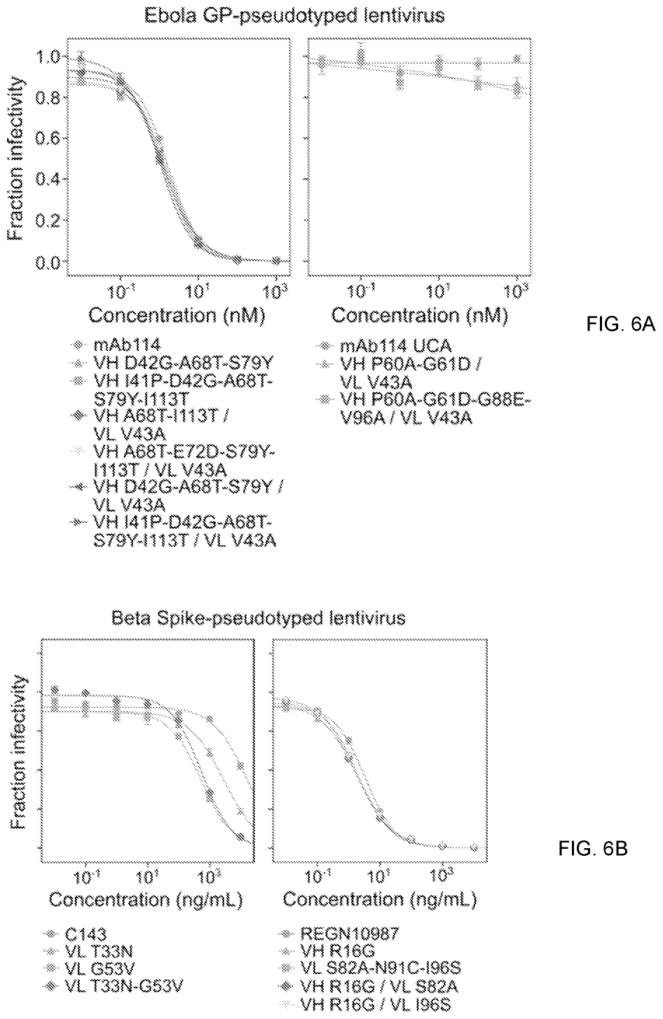

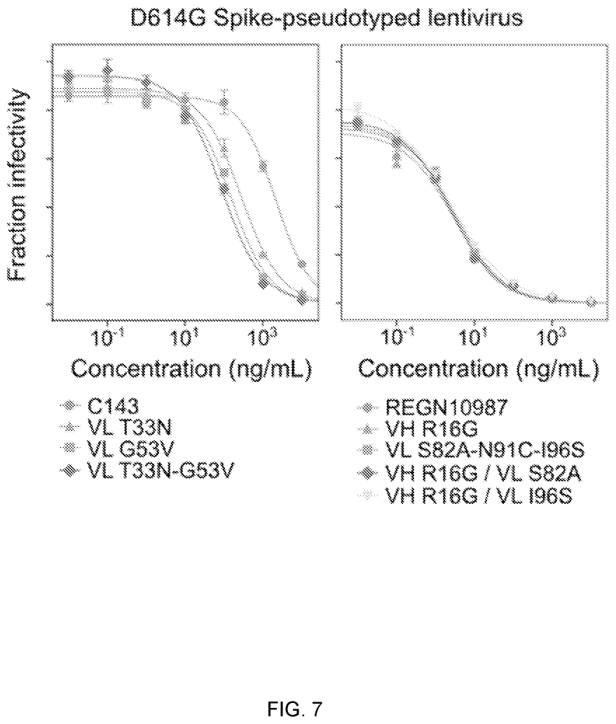

The affinity-matured variants were also assayed for improved viral neutralization. Affinity-enhancing variants of six antibodies were tested, and, in all cases, variants with significantly improved IC₅₀ values were observed (Bonferroni-corrected, one-sided t-test P<0.05), including a 1.5-fold improvement for the best mAb114 variant against ebola-pseudovirus, a 2.0-fold improvement for the best REGN10987 variant against SARS-CoV-2 Beta-pseudovirus, a 31-fold improvement for the best C143 variant against Beta-pseudovirus, a 12-fold improvement for the best LY-1404 variant against BQ.1.1 pesudovirus, and an 11.4-fold improvement for the best SA58 variant against BQ.1.1 pesudovirus (FIG. 5A and FIGS. 6A-7; Table 9, Table 12, and Tables 13-15). Additionally, while the IC₅₀s of variants of mAb114 UCA are greater than the highest tested concentration, the affinity-matured variants demonstrate some neutralization at a >100-fold lower concentration compared to wildtype (FIGS. 6A-7). In general, change in binding affinity correlates well with change in neutralization (Spearman r=0.82, two-sided t-distribution P=1.9×10⁻⁴) (FIG. 5B). Given the limited number of variants tested, it is also noted that alternative versions of the directed evolution campaigns described herein could have selected explicitly for neutralization.

### Example 4. Originality of Affinity-Enhancing Mutations

While the ability to find any affinity-enhancing mutations is itself useful for engineering applications, it was also of interest whether some of these mutations demonstrate scientific “originality.” Originality was quantified by computing the frequency that a given residue is observed in nature (see Example 1), where a mutation to a rarely-observed residue indicates that the model learns patterns that go beyond its literal training dataset. While many affinity-enhancing mutations are indeed observed at high frequency in both the model's training data [17] and in a database of antibody sequences [30], other mutations demonstrate greater originality. For example, in the MEDI8852 UCA trajectory, the VL G95P mutation (FIGS. 2A-4C) involves changing a glycine observed in 99% of natural antibody sequences to a proline observed in <1% of natural sequences. Overall, five out of 32 affinity-enhancing mutations (˜16%) involve a rare or uncommon mutant residue (Table 14). These results indicate that the language models learn both the “easy” evolutionary rules involving high-frequency residues, as well as more complex rules that would not be obvious from a multiple sequence alignment. Conceptually, these low-frequency, high-affinity mutations are analogous to examples in other disciplines where an artificial-intelligence program occasionally makes unusual but advantageous choices (for example, unintuitive game-playing decisions [31]), and likewise may be worth further study.

TABLE 16
Originality of affinity-enhancing mutations.

| | | UniRef90 (training dataset) | | | | | abYsis | | | |
|---|---|---|---|---|---|---|---|---|---|---|
| | Affinity- | Wildtype | Mutant | | Top | | Wildtype | Mutant | | Top |
| | enhancing | residue | residue | Top | residue | | residue | residue | Top | residue |
| Antibody | mutation | frequency | frequency | residue | frequency | Notes | frequency | frequency | residue | frequency |
| MEDI8852 | VH E65P | 6% | 32% | D | 35% | | 14% | 22% | D | 32% |
| | VH M117Y | <1% | 59% | — | — | | <1% | 49% | — | — |
| MEDI8852 | VH K58S | 8% | 3% | T | 67% | Uncommon | 2% | 30% | G | 35% |
| UCA | | | | | | to rare | | | | |
| | VH V65P | 1% | 32% | D | 36% | | 2% | 22% | D | 32% |
| | VL G95P | 44% | 2% | G | 44% | Common to | 99% | <1% | G | 99% |
| | | | | | | rare | | | | |
| mAb114 | VH M31S | <1% | 45% | — | — | | <1% | 48% | — | — |
| | VH A68T | 1% | 88% | — | — | | 1% | 85% | — | — |
| | VH E72D | 4% | 91% | — | — | | 2% | 93% | — | — |
| | VH S79Y | 8% | 74% | — | — | | 16% | 67% | — | — |
| | VH I113T | 2% | 69% | — | — | | 2% | 89% | — | — |
| | VL V43A | 4% | 50% | — | — | | 2% | 57% | — | — |
| mAb114 | VH T41P | 1% | 94% | — | — | | 1% | 92% | — | — |
| UCA | VH G61D | 2% | 57% | — | — | | 1% | 32% | — | — |
| | VH E72D | 4% | 92% | — | — | | 2% | 93% | — | — |
| | VH G88E | 1% | 85% | — | — | | 2% | 67% | — | — |
| | VH V96A | 6% | 77% | — | — | | 5% | 85% | — | — |
| | VL V43A | 4% | 50% | — | — | | 2% | 57% | — | — |
| S309 | VH G79A | 1% | 37% | — | — | | <1% | 34% | — | — |
| | VH R87T | 37% | 30% | R | 37% | | 39% | 41% | — | — |
| | VL T28S | 1% | 54% | — | — | | 2% | 77% | — | — |
| REGN10987 | VH R16G | 5% | 68% | — | — | | 6% | 31% | — | — |
| | VL N91C | 2% | 2% | Q | 39% | Rare to | 1% | 4% | Q | 52% |
| | | | | | | rare | | | | |
| C143 | VH V29F | 3% | 78% | — | — | | 4% | 69% | — | — |
| | VH L51Y | 4% | 18% | — | — | | 1% | <1% | I | 85% |
| | VH A77T | 1% | 56% | — | — | | <1% | 62% | — | — |
| | VH G91A | 4% | 95% | — | — | | 2% | 97% | — | — |
| | VL N27S | 11% | 53% | — | — | | 3% | 77% | — | — |
| | VL T33N | 2% | 44% | — | — | | 2% | 36% | — | — |
| | VL L34Y | 4% | 34% | — | — | | 2% | 59% | — | — |
| | VL Y41H | <1% | 6% | K | 59% | Rare to | <1% | 5% | K | 67% |
| | | | | | | uncommon | | | | |
| | VL G53V | 2% | 10% | A | 30% | Rare to | 4% | 12% | A | 40% |
| | | | | | | uncommon | | | | |
| | VL A96S | 2% | 57% | — | — | | 2% | 36% | — | — |

In Table 16, each row corresponds to a mutation that enhances the binding affinity of its corresponding variant IgG or Fab, and some of which also enhance affinity in combination with other mutations. Mutational frequencies were computed using two datasets, UniRef90 and abYsis (see Example 1); UniRef90 was the sequence database used to train the language models in the algorithm, and abYsis is a separate, curated database of natural antibody sequences. The “wildtype residue frequency” indicates the percentage of sequences in a multiple sequence alignment with the same residue as wildtype at the given position; the “mutant residue frequency” is the same statistic except for the mutant residue. The “top residue” indicates the amino acid with the highest frequency observed at the given site, the “top residue frequency” indicates the percentage of sequences that contain the top residue at the given site, and dashes indicate settings in which the mutant residue is also the top residue. Mutations with frequencies up to 5% are considered “rare,” those with frequencies above 5% and up to 10% are considered “uncommon,” and those above 10% are considered “common.” Bold text indicates mutations to rare residues according to frequency information from either UniRef90 or abYsis

### Example 5. Same Language Models Guide Efficient Evolution Across Diverse Natural Proteins

Given the success of general protein language models at guiding antibody evolution, it was also tested how well the same models could acquire high-fitness variants across a range of protein families. Previous work has demonstrated that language-model likelihoods have good correlation with experimental phenotypes from high-throughput assays measuring 103 to 104 variants [15]. Previous computational simulations have also indicated that these models can help bias multi-round exploration away from large regions of a sequence landscape with zero or very low fitness [7], [33], [34].

Here, it was observed that the same models used to affinity-mature antibodies can also guide efficient evolution when measuring only a small number (˜10¹) of variants according to diverse definitions of extrinsic fitness including antibiotic resistance, cancer drug resistance, enzyme activity, or viral replication fitness [35]. Across multiple proteins, the highest-likelihood mutations are significantly enriched for high-fitness variants (FIG. 8A, Table 17), and in all cases, high-fitness variants make up 12% or more of the high-likelihood variants. In the most pronounced example, ampicillin resistance mutations to β-lactamase, which make up just 0.7% of the full mutational space, make up 40% of language-model-recommended mutations. It is emphasized that the same algorithm and language models were used as for the affinity-maturation experiments, only having a different wildtype sequence supplied (for example, the sequence of 3-lactamase instead of an antibody VH). These results suggest that the evolutionary efficiency observed for affinity-maturation of human IgGs also generalizes to diverse natural settings.

In Table 17, each row corresponds to a protein tested via a high-throughput scanning mutagenesis assay that measures various notions of protein fitness, which are summarized in the “Fitness setting” column. All assays involve deep mutational scans that profile 90% or more of the full single-residue mutational landscape except for that of PafA, which mutates every residue to either a glycine or a valine. The cutoff indicates the study-specific criterion for determining a high-fitness mutation. The “Sample size” indicates the number of acquired mutations (| [symbol] |) and “Sample successes” indicates the number of those mutations with high fitness according to the cutoff. The “Population size” indicates the number of variants profiled in the scanning mutagenesis assay, where “Population successes” indicates the number of those variants with high fitness according to the cutoff. “Hit rate” indicates the percentage fraction of high-fitness variants among the acquired mutations (sample successes divided by sample size) whereas “Background” indicates the percentage fraction of high-fitness variants among the full, measured mutational space (population successes divided by population size). The hypergeometric P value computes enrichment of high-fitness mutations among the acquired mutations by assuming that the number of sample successes has a hypergeometric null distribution with parameters given by the other values (sample size, population successes, and population size); bold text indicates a one-sided, hypergeometric P value of less than 0.05.

TABLE 17
Enrichment of high-fitness mutations among language-model-recommended mutations.

| | | | | | | | | Popu- | | | | |
|---|---|---|---|---|---|---|---|---|---|---|---|---|
| | | | | | | Sample | | lation | Popu- | Hit | Back- | Hypergeo- |
| | | | Fitness | | Cut- | suc- | Sample | suc- | lation | rate | ground | metric |
| Protein | Reference | Organism | setting | Experiment | off | cesses | size | cesses | size | (%) | (%) | P |
| ADRB2 | Jones et | Human | Signal | Deep | >2.8 | 2 | 9 | 915 | 7847 | 22.2 | 11.7 | 0.28 |
| | al., 2019 | | transduction + | mutational | | | | | | | | |
| | [48] | | pathway | scan | | | | | | | | |
| | | | reporter | | | | | | | | | |
| β- | Stiffler et | Bacteria | Antibiotic | Deep | >0.1 | 4 | 10 | 39 | 5434 | 40.0 | 0.7 | 4.6 × 10⁻⁷ |
| lactamase | al, 2015 | | resistance | mutational | | | | | | | | |
| | [49] | | (ampicillin, | scan | | | | | | | | |
| | | | 2500 ug/mL) | | | | | | | | | |
| Env | Haddox et | Virus | Viral | Deep | >0.1 | 7 | 37 | 749 | 16359 | 18.9 | 4.6 | 0.0013 |
| | al., 2016 | | replication | mutational | | | | | | | | |
| | [50] | | fitness | scan | | | | | | | | |
| HA H1 | Doud and | Virus | Viral | Deep | >0.1 | 5 | 32 | 646 | 10736 | 15.6 | 6.0 | 0.041 |
| | Bloom, | | replication | mutational | | | | | | | | |
| | 2016 [51] | | fitness | scan | | | | | | | | |
| HA H3 | Lee et al., | Virus | Viral | Deep | >0.1 | 5 | 16 | 715 | 10754 | 31.3 | 6.6 | 0.0030 |
| | 2018 [52] | | replication | mutational | | | | | | | | |
| | | | fitness | scan | | | | | | | | |
| infA | Kelsic et | Bacteria | Competitive | Deep | >0.98 | 4 | 10 | 306 | 1369 | 40.0 | 22.4 | 0.17 |
| | al., 2016 | | growth | mutational | | | | | | | | |
| | [53] | | | scan | | | | | | | | |
| MAPK1 | Brenan et | Human | Competitive | Deep | <−4 | 4 | 13 | 584 | 6841 | 30.8 | 8.5 | 0.020 |
| | al., 2016 | | growth | mutational | | | | | | | | |
| | [54] | | (SCH) | scan | | | | | | | | |
| P53 | Giacomelli | Human | Competitive | Deep | >1 | 2 | 17 | 906 | 7467 | 11.8 | 12.1 | 0.63 |
| | et al, 2018 | | growth | mutational | | | | | | | | |
| | [55] | | (etoposide) | scan | | | | | | | | |
| PafA | Markin et | Bacteria | K_{cat}/K_{M} | Scanning | P < 0.01, | 2 | 10 | 35 | 1041 | 20.0 | 3.4 | 0.042 |
| | al., 2021 | | | mutagenesis | faster | | | | | | | |
| | [41] | | | | than WT | | | | | | | |

### SELECTED REFERENCES CITED IN THIS DISCLOSURE

[1] R. Dawkins, Climbing Mount Improbable. 1997.
[2] M. Gerstung et al., “The evolutionary history of 2,658 cancers,” Nature, vol. 578, no. 7793, pp. 122-128, February 2020, doi: 10.1038/s41586-019-1907-7.
[3] S. B. Joseph, R. Swanstrom, A. D. M. Kashuba, and M. S. Cohen, “Bottlenecks in HIV-1 transmission: insights from the study of founder viruses,” Nature Reviews Microbiology, vol. 13, no. 7, pp. 414-425, July 2015, doi: 10.1038/nrmicro3471.
[4] G. D. Victora and M. C. Nussenzweig, “Germinal Centers,” Annual Review of Immunology, vol. 30, no. 1, pp. 429-457, April 2012, doi: 10.1146/annurev-immunol-020711-075032.
[5] S. C. Morris, Life's solution: Inevitable humans in a lonely universe. 2003. doi: 10.1017/CB09780511535499.
[6] S. J. Gould, Wonderful Life: The Burgess Shale and the Nature of History. WW Norton & Company, 1990. doi: 10.1016/0169-5347(90)90105-m.
[7] B. J. Wittmann, Y. Yue, and F. H. Arnold, “Informed training set design enables efficient machine learning-assisted directed protein evolution,” Cell Systems, vol. 12, no. 11, pp. 1026-1045.e7, November 2021, doi: 10.1016/j.cels.2021.07.008.
[8] B. L. Hie, K. K. Yang, and P. S. Kim, “Evolutionary velocity with protein language models predicts evolutionary dynamics of diverse proteins,” Cell Systems, February 2022, doi: 10.1016/j.cels.2022.01.003.
[9] A. Wellner et al., “Rapid generation of potent antibodies by autonomous hypermutation in yeast,” Nature Chemical Biology, vol. 17, no. 10, pp. 1057-1064, October 2021, doi: 10.1038/s41589-021-00832-4.
[10] T. Bepler and B. Berger, “Learning the protein language: Evolution, structure, and function,” Cell Systems, vol. 12, no. 6, Jun. 2021, doi: 10.1016/j.cels.2021.05.017.
[11] T. Bepler and B. Berger, “Learning protein sequence embeddings using information from structure,” in 7th International Conference on Learning Representations, 2019, vol. arXiv, no. cs.LG, p. 1902.08661.
[12] B. Hie, E. Zhong, B. Berger, and B. Bryson, “Learning the language of viral evolution and escape,” Science, vol. 371, no. 6526, pp. 284-288, 2021.
[13] E. C. Alley, G. Khimulya, S. Biswas, M. AlQuraishi, and G. M. Church, “Unified rational protein engineering with sequence-based deep representation learning,” Nature Methods, vol. 16, no. 12, pp. 1315-1322, 2019, doi: 10.1038/s41592-019-0598-1.
[14] A. Rives et al., “Biological structure and function emerge from scaling unsupervised learning to 250 million protein sequences,” Proceedings of the National Academy of Sciences, vol. 118, no. 15, p. e2016239118, 2021, doi: 10.1073/pnas.2016239118.
[15] J. Meier, R. Rao, R. Verkuil, J. Liu, T. Sercu, and A. Rives, “Language models enable zero-shot prediction of the effects of mutations on protein function,” bioRxiv, p. 10.1101/2021.07.09.450648, 2021.
[16] R. Rao et al., “Evaluating Protein Transfer Learning with TAPE,” Advances in Neural Information Processing Systems, vol. 32, pp. 9686-9698, 2019.
[17] B. E. Suzek, H. Huang, P. McGarvey, R. Mazumder, and C. H. Wu, “UniRef: Comprehensive and non-redundant UniProt reference clusters,” Bioinformatics, vol. 23, no. 10, pp. 1282-1288, 2007, doi: 10.1093/bioinformatics/btm098.
[18] J. A. Ruffolo, J. J. Gray, and J. Sulam, “Deciphering antibody affinity maturation with language models and weakly supervised learning,” arXiv, vol. cs.LG, no. [q-bio.BM], 2021.
[19] R. W. Shuai, J. A. Ruffolo, and J. J. Gray, “Generative Language Modeling for Antibody Design,” bioRxiv, no. 10.1101/2021.12.13.472419, 2021.
[20] K. Saka et al., “Antibody design using LSTM based deep generative model from phage display library for affinity maturation,” Scientific Reports, vol. 11, no. 1, p. 5852, December 2021, doi: 10.1038/s41598-021-85274-7.
[21] J.-E. Shin et al., “Protein design and variant prediction using autoregressive generative models,” Nature Communications, vol. 12, no. 1, p. Article number: 2403, 2021.
[22] N. L. Kallewaard et al., “Structure and Function Analysis of an Antibody Recognizing All Influenza A Subtypes,” Cell, vol. 166, no. 3, pp. 596-608, July 2016, doi: 10.1016/j.cell.2016.05.073.
[23] D. Corti et al., “Protective monotherapy against lethal Ebola virus infection by a potently neutralizing antibody,” Science, vol. 351, no. 6279, pp. 1339-1342, March 2016, doi: 10.1126/science.aad5224.
[24] R. Copin et al., “The monoclonal antibody combination REGEN-COV protects against SARS-CoV-2 mutational escape in preclinical and human studies,” Cell, vol. 184, no. 15, pp. 3949-3961.e11, July 2021, doi: 10.1016/j.cell.2021.06.002.
[25] D. Pinto et al., “Cross-neutralization of SARS-CoV-2 by a human monoclonal SARS-CoV antibody,” Nature, vol. 583, no. 7815, pp. 290-295, July 2020, doi: 10.1038/s41586-020-2349-y.
[26] K. K. Yang, Z. Wu, and F. H. Arnold, “Machine-learning-guided directed evolution for protein engineering,” Nature Methods, vol. 16, no. 8, pp. 687-694, 2019, doi: 10.1038/s41592-019-0496-6.
[27] B. L. Hie and K. K. Yang, “Adaptive machine learning for protein engineering,” Current Opinion in Structural Biology, vol. 72, pp. 145-152, February 2022, doi: 10.1016/j.sbi.2021.11.002.
[28] E. Alexander et al., “Antibody therapies for SARS-CoV-2 infection,” WO2021252878A1, 2021
[29] F. Muecksch et al., “Affinity maturation of SARS-CoV-2 neutralizing antibodies confers potency, breadth, and resilience to viral escape mutations,” Immunity, vol. 54, no. 8, pp. 1853-1868.e7, August 2021, doi: 10.1016/j.immuni.2021.07.008.
[30] M. B. Swindells et al., “abYsis: Integrated Antibody Sequence and Structure-Management, Analysis, and Prediction,” Journal of Molecular Biology, vol. 429, no. 3, pp. 356-364, February 2017, doi: 10.1016/j.jmb.2016.08.019.
[31] D. Silver et al., “Mastering the game of Go with deep neural networks and tree search,” Nature, vol. 529, no. 7587, pp. 484-489, 2016, doi: 10.1038/nature16961.
[32] M. I. J. Raybould et al., “Thera-SAbDab: the Therapeutic Structural Antibody Database,” Nucleic Acids Research, vol. 48, no. D1, pp. D383-D388, January 2020, doi: 10.1093/nar/gkz827.
[33] C. Hsu, H. Nisonoff, C. Fannjiang, and J. Listgarten, “Learning protein fitness models from evolutionary and assay-labeled data,” Nature Biotechnology, January 2022, doi: 10.1038/s41587-021-01146-5.
[34] B. Hie, B. D. Bryson, and B. Berger, “Leveraging Uncertainty in Machine Learning Accelerates Biological Discovery and Design,” Cell Systems, vol. 11, pp. 461-477, 2020, doi: 10.1016/j.cels.2020.09.007.
[35] B. J. Livesey and J. A. Marsh, “Using deep mutational scanning to benchmark variant effect predictors and identify disease mutations,” Molecular Systems Biology, vol. 16, no. 7, p. e9380, 2020, doi: 10.15252/msb.20199380.
[36] A. J. Riesselman, J. B. Ingraham, and D. S. Marks, “Deep generative models of genetic variation capture the effects of mutations,” Nature Methods, vol. 15, no. 10, pp. 816-822, 2018, doi: 10.1038/s41592-018-0138-4.
[37] J. Frazer et al., “Disease variant prediction with deep generative models of evolutionary data,” Nature, vol. 599, no. 7883, pp. 91-95, November 2021, doi: 10.1038/s41586-021-04043-8.
[38] T. A. Hopf et al., “Mutation effects predicted from sequence co-variation,” Nature Biotechnology, vol. 35, no. 2, pp. 128-135, 2017, doi: 10.1038/nbt.3769.
[39] H. Zhao, L. Giver, Z. Shao, J. A. Affholter, and F. H. Arnold, “Molecular evolution by staggered extension process (StEP) in vitro recombination,” Nature Biotechnology, vol. 16, no. 3, pp. 258-261, March 1998, doi: 10.1038/nbt0398-258.
[40] D. M. Mason et al., “Optimization of therapeutic antibodies by predicting antigen specificity from antibody sequence via deep learning,” Nature Biomedical Engineering, vol. 5, no. 6, June 2021, doi: 10.1038/s41551-021-00699-9.
[41] C. J. Markin et al., “Revealing enzyme functional architecture via high-throughput microfluidic enzyme kinetics,” Science, vol. 373, no. 6553, July 2021, doi: 10.1126/science.abf8761.
[42] S. Biswas, G. Khimulya, E. C. Alley, K. M. Esvelt, and G. M. Church, “Low-N protein engineering with data-efficient deep learning,” Nature Methods, vol. 18, no. 4, 2021, doi: 10.1038/s41592-021-01100-y.
[43] S. Sledzieski, R. Singh, L. Cowen, and B. Berger, “D-SCRIPT translates genome to phenome with sequence-based, structure-aware, genome-scale predictions of protein-protein interactions,” Cell Systems, vol. 12, no. 10, pp. 969-982.e6, October 2021, doi: 10.1016/j.cels.2021.08.010.
[44] M. L. Bileschi et al., “Using deep learning to annotate the protein universe,” Nature Biotechnology, February 2022, doi: 10.1038/s41587-021-01179-w.
[45] T. M. Chidyausiku et al., “De Novo Design of Immunoglobulin-like Domains,” bioRxiv, no. 10.1101/2021.12.20.472081, 2021.
[46] K. H. D. Crawford et al., “Protocol and Reagents for Pseudotyping Lentiviral Particles with SARS-CoV-2 Spike Protein for Neutralization Assays,” Viruses, vol. 12, no. 5, p. 513, May 2020, doi: 10.3390/v12050513.
[47] T. F. Rogers et al., “Isolation of potent SARS-CoV-2 neutralizing antibodies and protection from disease in a small animal model,” Science, vol. 369, no. 6506, pp. 956-963, August 2020, doi: 10.1126/science.abc7520.
[48] E. M. Jones et al., “Structural and functional characterization of G protein-coupled receptors with deep mutational scanning,” eLife, vol. 9, October 2020, doi: 10.7554/eLife.54895.
[49] M. A. Stiffler, D. R. Hekstra, and R. Ranganathan, “Evolvability as a Function of Purifying Selection in TEM-1 β-Lactamase,” Cell, vol. 160, no. 5, pp. 882-892, February 2015, doi: 10.1016/j.cell.2015.01.035.
[50] H. K. Haddox, A. S. Dingens, and J. D. Bloom, “Experimental Estimation of the Effects of All Amino-Acid Mutations to HIV's Envelope Protein on Viral Replication in Cell Culture,” PLOS Pathogens, vol. 12, no. 12, p. e1006114, December 2016, doi: 10.1371/journal.ppat.1006114.
[51] M. B. Doud and J. D. Bloom, “Accurate measurement of the effects of all amino-acid mutations on influenza hemagglutinin,” Viruses, vol. 8, no. 6, p. 155, 2016, doi: 10.3390/v8060155.
[52] J. M. Lee et al., “Deep mutational scanning of hemagglutinin helps predict evolutionary fates of human H3N2 influenza variants,” Proceedings of the National Academy of Sciences, vol. 115, no. 35, August 2018, doi: 10.1073/pnas.1806133115.
[53] E. D. Kelsic, H. Chung, N. Cohen, J. Park, H. H. Wang, and R. Kishony, “RNA Structural Determinants of Optimal Codons Revealed by MAGE-Seq,” Cell Systems, vol. 3, no. 6, pp. 563-571.e6, December 2016, doi: 10.1016/j.cels.2016.11.004.
[54] L. Brenan et al., “Phenotypic Characterization of a Comprehensive Set of MAPK1/ERK2 Missense Mutants,” Cell Reports, vol. 17, no. 4, pp. 1171-1183, October 2016, doi: 10.1016/j.celrep.2016.09.061.
[55] A. O. Giacomelli et al., “Mutational processes shape the landscape of TP53 mutations in human cancer,” Nature Genetics, vol. 50, no. 10, pp. 1381-1387, October 2018, doi: 10.1038/s41588-018-0204-y.
[56] K. Westendorf et al., “LY-CoV1404 (bebtelovimab) potently neutralizes SARS-CoV-2 variants.” Cell Rep. 2022 vol. 39, no. 7, 110812. doi: 10.1016/j.celrep.2022.110812.
[57] “FACT SHEET FOR HEALTHCARE PROVIDERS: EMERGENCY USE AUTHORIZATION FOR BEBTELOVIMAB” Federal Drug Administration, November 2022.
[58] Y. Cao et al., “BA.2.12.1, BA.4 and BA.5 escape antibodies elicited by Omicron infection.” Nature 2022 vol. 608, pp. 593-602. doi:10.1038/s41586-022-04980-y.
[59] R. Song et al., “Post-Exposure Prophylaxis with SA58 (anti-COVID-19 monoclonal antibody) Nasal Spray for the prevention of symptomatic Coronavirus Disease 2019 in healthy adult workers: A randomized, single-blind, placebo-controlled clinical study.” MedRxiv 2022. doi: 10.1101/2022.12.28.22283666.

1. An isolated antibody or antigen-binding portion thereof comprising one of:
I (a) a heavy chain variable region comprising
(i) a CDRH1 comprising at least 90% identity to SEQ ID NO: 15;
(ii) a CDRH2 comprising at least 90% identity to SEQ ID NO: 16; and
(iii) a CDRH3 comprising at least 90% identity to SEQ ID NO: 17; and
(b) a light chain variable region comprising
(i) a CDRL1 comprising at least 90% identity to SEQ ID NO:35;
(ii) a CDRL2 comprising at least 90% identity to SEQ ID NO:36; and
(iii) a CDRL3 comprising at least 90% identity to SEQ ID NO:37,
wherein the heavy chain variable region comprises an amino acid residue substitution at at least one of positions 124, D27, S44, T53, E65, N74, P75, or M117, wherein the positions are numbered with respect to SEQ ID NO:1, and/or
wherein the light chain variable region comprises an amino acid residue substitution at at least one of positions T25, L29, T33, G55, R92, and G95, wherein the positions are numbered with respect to SEQ ID NO:2;
(II) (a) a heavy chain variable region comprising
(i) a CDRH1 comprising at least 90% identity to SEQ ID NO:18;
(ii) a CDRH2 comprising at least 90% identity to SEQ ID NO: 19; and
(iii) a CDRH3 comprising at least 90% identity to SEQ ID NO:20; and
(b) a light chain variable region comprising
(i) a CDRL1 comprising at least 90% identity to SEQ ID NO:38;
(ii) a CDRL2 comprising at least 90% identity to SEQ ID NO:39; and
(iii) a CDRL3 comprising at least 90% identity to SEQ ID NO:37,
wherein the heavy chain variable region comprises an amino acid residue substitution at at least one of positions S44, T53, K58, V65, N74, and P75, wherein the positions are numbered with respect to SEQ ID NO:3, and/or
wherein the light chain variable region comprises an amino acid residue substitution at at least one of positions N34 and G95, wherein the positions are numbered with respect to SEQ ID NO:4:
(III) (a) a heavy chain variable region comprising
(i) a CDRH1 comprising at least 90% identity to SEQ ID NO:21;
(ii) a CDRH2 comprising at least 90% identity to SEQ ID NO:22; and
(iii) a CDRH3 comprising at least 90% identity to SEQ ID NO:23; and
(b) a light chain variable region comprising
(i) a CDRL1 comprising at least 90% identity to SEQ ID NO:40;
(ii) a CDRL2 comprising at least 90% identity to SEQ ID NO:41; and
(iii) a CDRL3 comprising at least 90% identity to SEQ ID NO:42,
wherein the heavy chain variable region comprises an amino acid residue substitution at at least one of positions M31, 141, D42, A68, E72, S79, and 1113, wherein the positions are numbered with respect to SEQ ID NO:5, and/or
wherein the light chain variable region comprises an amino acid residue substitution at at least one of positions 119, F29, V43, S49, H70, and N90, wherein the positions are numbered with respect to SEQ ID NO:6;
(IV) (a) a heavy chain variable region comprising
(i) a CDRH1 comprising at least 90% identity to SEQ ID NO:24;
(ii) a CDRH2 comprising at least 90% identity to SEQ ID NO:25; and
(iii) a CDRH3 comprising at least 90% identity to SEQ ID NO:23; and
(b) a light chain variable region comprising
(i) a CDRL1 comprising at least 90% identity to SEQ ID NO:43;
(ii) a CDRL2 comprising at least 90% identity to SEQ ID NO:44; and
(iii) a CDRL3 comprising at least 90% identity to SEQ ID NO:45,
wherein the heavy chain variable region comprises an amino acid residue substitution at at least one of positions T41, A54, P60, G61, E72, G88, and V96, wherein the positions are numbered with respect to SEQ ID NO:7, and/or
wherein the light chain variable region comprises an amino acid residue substitution at at least one of positions V43 and K90, wherein the positions are numbered with respect to SEQ ID NO:8:
(V) (a) a heavy chain variable region comprising
(i) a CDRH1 comprising at least 90% identity to SEQ ID NO:26;
(ii) a CDRH2 comprising at least 90% identity to SEQ ID NO:27; and
(iii) a CDRH3 comprising at least 90% identity to SEQ ID NO:28; and
(b) a light chain variable region comprising
(i) a CDRL1 comprising at least 90% identity to SEQ ID NO:46;
(ii) a CDRL2 comprising at least 90% identity to SEQ ID NO:47; and
(iii) a CDRL3 comprising at least 90% identity to SEQ ID NO:48,
wherein the heavy chain variable region comprises an amino acid residue substitution at at least one of positions P28, T77, G79, R84, R85, and R87, wherein the positions are numbered with respect to SEQ ID NO:9, and/or
wherein the light chain variable region comprises an amino acid residue substitution at at least one of positions T28, T32, S95, and L96, wherein the positions are numbered with respect to SEQ ID NO:10:
(VI) (a) a heavy chain variable region comprising
(i) a CDRH1 comprising at least 90% identity to SEQ ID NO:29;
(ii) a CDRH2 comprising at least 90% identity to SEQ ID NO:30; and
(iii) a CDRH3 comprising at least 90% identity to SEQ ID NO:31; and
(b) a light chain variable region comprising
(i) a CDRL1 comprising at least 90% identity to SEQ ID NO:49;
(ii) a CDRL2 comprising at least 90% identity to SEQ ID NO:50; and
(iii) a CDRL3 comprising at least 90% identity to SEQ ID NO:51,
wherein the heavy chain variable region comprises an amino acid residue substitution at at least one of positions R16, S98, and V108, wherein the positions are numbered with respect to SEQ ID NO:11, and/or
wherein the light chain variable region comprises an amino acid residue substitution at at least one of positions S82, N91, L93, and 196, wherein the positions are numbered with respect to SEQ ID NO:12:
(VII) (a) a heavy chain variable region comprising
(i) a CDRH1 comprising at least 90% identity to SEQ ID NO:32;
(ii) a CDRH2 comprising at least 90% identity to SEQ ID NO:33; and
(iii) a CDRH3 comprising at least 90% identity to SEQ ID NO:34; and
(b) a light chain variable region comprising
(i) a CDRL1 comprising at least 90% identity to SEQ ID NO: 52;
(ii) a CDRL2 comprising at least 90% identity to SEQ ID NO:53; and
(iii) a CDRL3 comprising at least 90% identity to SEQ ID NO:54,
wherein the heavy chain variable region comprises an amino acid residue substitution at at least one of positions V29, K32, L51, D57, A77, and G91, wherein the positions are numbered with respect to SEQ ID NO:13, and/or
wherein the light chain variable region comprises an amino acid residue substitution at at least one of positions N27, T33, L34, Y41, G53, S57, G82, and A96, wherein the positions are numbered with respect to SEQ ID NO:14:
(VIII) (a) a heavy chain variable region comprising
(i) a CDRH1 comprising at least 90% identity to SEQ ID NO:62;
(ii) a CDRH2 comprising at least 90% identity to SEQ ID NO:63; and
(iii) a CDRH3 comprising at least 90% identity to SEQ ID NO:64; and
(b) a light chain variable region comprising
(i) a CDRL1 comprising at least 90% identity to SEQ ID NO:68;
(ii) a CDRL2 comprising at least 90% identity to SEQ ID NO:69; and
(iii) a CDRL3 comprising at least 90% identity to SEQ ID NO:70,
wherein the heavy chain variable region comprises an amino acid residue substitution at at least one of positions D88, V90, S62, V81, F24, I31, H99, T79, and 1105, wherein the positions are numbered with respect to SEQ ID NO:58, and/or
wherein the light chain variable region comprises an amino acid residue substitution at at least one of positions A98, Q39, T5, K47, F51, K44, M49, E85, and Q6, wherein the positions are numbered with respect to SEQ ID NO:59; or
(IX) (a) a heavy chain variable region comprising
(i) a CDRH1 comprising at least 90% identity to SEQ ID NO:65;
(ii) a CDRH2 comprising at least 90% identity to SEQ ID NO:66; and
(iii) a CDRH3 comprising at least 90% identity to SEQ ID NO:67; and
(b) a light chain variable region comprising
(i) a CDRL1 comprising at least 90% identity to SEQ ID NO:71;
(ii) a CDRL2 comprising at least 90% identity to SEQ ID NO:72; and
(iii) a CDRL3 comprising at least 90% identity to SEQ ID NO:73,
wherein the heavy chain variable region comprises an amino acid residue substitution at at least one of positions T53, A61, and E10, wherein the positions are numbered with respect to SEQ ID NO:60, and/or
wherein the light chain variable region comprises an amino acid residue substitution at at least one of positions N95, 585, 554, and M4, wherein the positions are numbered with respect to SEQ ID NO:61.

2. The isolated antibody or antigen-binding portion thereof of claim 1, wherein the isolated antibody or antigen-binding portion thereof comprises (I), and the heavy chain variable region comprises amino acid residue substitutions at positions E65 and M117, wherein the positions are numbered with respect to SEQ ID NO:1.

3-5. (canceled)

6. The isolated antibody or antigen-binding portion thereof of claim 1, wherein the isolated antibody or antigen-binding portion thereof comprises (II), and
i. the heavy chain variable region comprises amino acid residue substitutions at positions K58 and V65;
ii. the heavy chain variable region comprises amino acid residue substitutions at positions K58 and P75;
iii. the heavy chain variable region comprises amino acid residue substitutions at positions V65 and P75;
iv. the heavy chain variable region comprises amino acid residue substitutions at positions K58, V65, and P75;
v. the heavy chain variable region comprises an amino acid residue substitution at position K58 and the light chain variable region comprises an amino acid residue substitution at position G95;
vi. the heavy chain variable region comprises an amino acid residue substitution at position V65 and the light chain variable region comprises an amino acid residue substitution at position G95;
vii. the heavy chain variable region comprises an amino acid residue substitution at position P75 and the light chain variable region comprises an amino acid residue substitution at position G95;
viii. the heavy chain variable region comprises amino acid residue substitutions at positions K58 and V65 and the light chain variable region comprises an amino acid residue substitution at position G95;
ix. the heavy chain variable region comprises amino acid residue substitutions at positions K58 and P75 and the light chain variable region comprises an amino acid residue substitution at position G95;
x. the heavy chain variable region comprises amino acid residue substitutions at positions V65 and P75 and the light chain variable region comprises an amino acid residue substitution at position G95; or
xi. the heavy chain variable region comprises amino acid residue substitutions at positions K58, V65, and P75 and the light chain variable region comprises an amino acid residue substitution at position G95,
wherein the heavy chain variable region positions are numbered with respect to SEQ ID NO:3 and the light chain variable region positions are numbered with respect to SEQ ID NO:4.

7-9. (canceled)

10. The isolated antibody or antigen-binding portion thereof of claim 1, wherein the isolated antibody or antigen-binding portion thereof comprises (III), and
i. the heavy chain variable region comprises amino acid residue substitutions at positions D42, A68, and S79;
ii. the heavy chain variable region comprises amino acid residue substitutions at positions 141, D42, A68, S79, and 1113;
iii. the heavy chain variable region comprises amino acid residue substitutions at positions A68 and 1113 and the light chain variable region comprises an amino acid residue substitution at position V43;
iv. the heavy chain variable region comprises amino acid residue substitutions at positions A68, E72, S79, and 1113 and the light chain variable region comprises an amino acid residue substitution at position V43;
v. the heavy chain variable region comprises amino acid residue substitutions at positions D42, A68, and S79 and the light chain variable region comprises an amino acid residue substitution at position V43; or
vi. the heavy chain variable region comprises amino acid residue substitutions at positions 141, D42, A68, S79, and 1113 and the light chain variable region comprises an amino acid residue substitution at position V43,
wherein the heavy chain variable region positions are numbered with respect to SEQ ID NO:5 and the light chain variable region positions are numbered with respect to SEQ ID NO:6.

11-13. (canceled)

14. The isolated antibody or antigen-binding portion thereof of claim 1, wherein the isolated antibody or antigen-binding portion thereof comprises (IV), and
i. the heavy chain variable region comprises amino acid residue substitutions at positions P60 and G61;
ii. the heavy chain variable region comprises amino acid residue substitutions at positions T41, P60, G61, E72, G88, and V96;
iii. the heavy chain variable region comprises an amino acid residue substitution at position G88 and the light chain variable region comprises an amino acid residue substitution at position V43;
iv. the heavy chain variable region comprises an amino acid residue substitution at position V96 and the light chain variable region comprises an amino acid residue substitution at position V43;
v. the heavy chain variable region comprises amino acid residue substitutions at positions P60 and G61 and the light chain variable region comprises an amino acid residue substitution at position V43;
vi. the heavy chain variable region comprises amino acid residue substitutions at positions P60, G61, G88, and V96 and the light chain variable region comprises an amino acid residue substitution at position V43; or
vii. the heavy chain variable region comprises amino acid residue substitutions at positions T41, P60, G61, E72, G88, and V96 and the light chain variable region comprises an amino acid residue substitution at position V43,
wherein the heavy chain variable region positions are numbered with respect to SEQ ID NO:7 and the light chain variable region positions are numbered with respect to SEQ ID NO:8.

15-17. (canceled)

18. The isolated antibody or antigen-binding portion thereof of claim 1, wherein the isolated antibody or antigen-binding portion thereof comprises (V), and
i. the heavy chain variable region comprises amino acid residue substitutions at positions T77, G79, and R84;
ii. the heavy chain variable region comprises amino acid residue substitutions at positions T77, G79, R84, and R85;
iii. the light chain variable region comprises amino acid residue substitutions at positions T28 and T32;
iv. the heavy chain variable region comprises an amino acid residue substitution at position G79 and the light chain variable region comprises an amino acid residue substitution at position T28;
v. the heavy chain variable region comprises an amino acid residue substitution at position R84 and the light chain variable region comprises an amino acid residue substitution at position T28;
vi. the heavy chain variable region comprises an amino acid residue substitution at position R87 and the light chain variable region comprises an amino acid residue substitution at position T28;
vii. the heavy chain variable region comprises amino acid residue substitutions at positions T77, G79, and R84 and the light chain variable region comprises an amino acid residue substitution at position T28;
viii. the heavy chain variable region comprises amino acid residue substitutions at positions T77, G79, and R84 and the light chain variable region comprises amino acid residue substitutions at positions T28 and T32; or
ix. the heavy chain variable region comprises amino acid residue substitutions at positions T77, G79, R84, and R85 and the light chain variable region comprises amino acid residue substitutions at positions T28 and T32,
wherein the heavy chain variable region positions are numbered with respect to SEQ ID NO:9 and the light chain variable region positions are numbered with respect to SEQ ID NO:10.

19-21. (canceled)

22. The isolated antibody or antigen-binding portion thereof of claim 1, wherein the isolated antibody or antigen-binding portion thereof comprises (VI), and
i. the heavy chain variable region comprises amino acid residue substitutions at positions R16 and V108;
ii. the light chain variable region comprises amino acid residue substitutions at positions S82, N91, and 196;
iii. the heavy chain variable region comprises an amino acid residue substitution at position R16 and the light chain variable region comprises an amino acid residue substitution at position S82;
iv. the heavy chain variable region comprises an amino acid residue substitution at position R16 and the light chain variable region comprises an amino acid residue substitution at position N91;
v. the heavy chain variable region comprises an amino acid residue substitution at position R16 and the light chain variable region comprises an amino acid residue substitution at position I96; or
vi. the heavy chain variable region comprises amino acid residue substitutions at positions R16 and V108 and the light chain variable region comprises amino acid residue substitutions at positions 582, N91, and I96,
wherein the heavy chain variable region positions are numbered with respect to SEQ ID NO:11 and the light chain variable region positions are numbered with respect to SEQ ID NO:12.

23-25. (canceled)

26. The isolated antibody or antigen-binding portion thereof of claim 1, wherein the isolated antibody or antigen-binding portion thereof comprises (VIII and
i. the heavy chain variable region comprises amino acid residue substitutions at positions L51, A77, and G91;
ii. the light chain variable region comprises amino acid residue substitutions at positions T33 and G53;
iii. the light chain variable region comprises amino acid residue substitutions at positions N27, T33, L34, and G53;
iv. the light chain variable region comprises amino acid residue substitutions at positions N27, T33, L34, Y41, G53, S57, and G82;
v. the heavy chain variable region comprises amino acid residue substitutions at positions L51, A77, and G91 and the light chain variable region comprises an amino acid residue substitution at position T33; or
vi. the heavy chain variable region comprises amino acid residue substitutions at positions L51, A77, and G91 and the light chain variable region comprises amino acid residue substitutions at positions N27, T33, L34, and G53,
wherein the heavy chain variable region positions are numbered with respect to SEQ ID NO:13 and the light chain variable region positions are numbered with respect to SEQ ID NO:14.

27-29. (canceled)

30. The isolated antibody or antigen-binding portion thereof of claim 1, wherein the isolated antibody or antigen-binding portion thereof comprises (VIII), and
i. the heavy chain variable region comprises amino acid residue substitutions at one or more positions D88, V90, S62, V81, F24, 131, H99, T70, and 1105;
ii. the heavy chain variable region comprises amino acid residue substitutions at positions D88, V90, S62, V81, F24, I31, H99, and T70;
iii. the heavy chain variable region comprises amino acid residue substitutions at positions D88, V90, S62, V81, F24, I31, and H99;
iv. the heavy chain variable region comprises amino acid residue substitutions at positions D88, V90, S62, V81, F24, I31, and T70;
v. the heavy chain variable region comprises amino acid residue substitutions at positions D88, V90, S62, V81, F24, I31, and T70;
vi. the heavy chain variable region comprises amino acid residue substitutions at positions D88, V90, S62, V81, I31, H99, and T70;
vii. the light chain variable region comprises amino acid residue substitutions at one or more positions A98I, Q39K, T5Q, K47E, F51Y, K44E, M49L, E85A, and Q6S;
viii. the heavy chain variable region comprises an amino acid residue substitution at position V90, and the light chain variable region comprises an amino acid residue substitution at position E85;
ix. the heavy chain variable region comprises an amino acid residue substitution at position S62, and the light chain variable region comprises an amino acid residue substitution at position E85;
x. the heavy chain variable region comprises an amino acid residue substitution at position T70, and the light chain variable region comprises an amino acid residue substitution at position E85;
xi. the heavy chain variable region comprises an amino acid residue substitution at position V90, and the light chain variable region comprises an amino acid residue substitution at position Q39;
xii. the heavy chain variable region comprises an amino acid residue substitution at position S62, and the light chain variable region comprises an amino acid residue substitution at position Q39; or
xiii. the heavy chain variable region comprises an amino acid residue substitution at position T70, and the light chain variable region comprises an amino acid residue substitution at position Q39,
wherein the heavy chain variable region positions are numbered with respect to SEQ ID NO:58 and the light chain variable region positions are numbered with respect to SEQ ID NO:59.

31-33. (canceled)

34. The isolated antibody or antigen-binding portion thereof of claim 1, wherein the isolated antibody or antigen-binding portion thereof comprises (IX), and
i. the heavy chain variable region comprises amino acid residue substitutions at one or more positions T53, A61, and E10;
ii. the light chain variable region comprises amino acid residue substitutions at one or more positions N95, S85, S54, and M4;
iii. the heavy chain variable region comprises an amino acid residue substitution at position T53, and the light chain variable region comprises an amino acid residue substitution at position N95;
iv. the heavy chain variable region comprises an amino acid residue substitution at position Q82, and the light chain variable region comprises an amino acid residue substitution at position N95;
v. the heavy chain variable region comprises an amino acid residue substitution at position A61, and the light chain variable region comprises an amino acid residue substitution at position N95;
vi. the heavy chain variable region comprises an amino acid residue substitution at position T53, and the light chain variable region comprises an amino acid residue substitution at position M4;
vii. the heavy chain variable region comprises an amino acid residue substitution at position Q82, and the light chain variable region comprises an amino acid residue substitution at position M4,
viii. the heavy chain variable region comprises an amino acid residue substitution at position A61, and the light chain variable region comprises an amino acid residue substitution at position M4, or
ix. the heavy chain variable region comprises an amino acid residue substitutions at positions A61 and T53,
wherein the heavy chain variable region positions are numbered with respect to SEQ ID NO:60 and the light chain variable region positions are numbered with respect to SEQ ID NO:61.

35-36. (canceled)

37. A recombinant nucleic acid molecule encoding the antibody or antigen binding portion thereof of claim 1.

38. (canceled)

39. A DNA construct comprising the recombinant nucleic acid molecule of claim 37 operably linked to a promoter that drives expression in a host cell.

40. A vector comprising the recombinant nucleic acid molecule of claim 37.

41. A host cell comprising the recombinant nucleic acid molecule of claim 37.

42-43. (canceled)

44. A composition comprising (a) the antibody or antigen binding portion thereof of claim 1; and (b) a pharmaceutically acceptable carrier.

45. A method of detecting a presence of a virus in a biological sample comprising:
(a) contacting said biological sample with the isolated antibody or antigen binding portion thereof of claim 1, and
(b) detecting an amount of binding of the isolated antibody or antigen binding portion thereof as a determination of the presence of the virus in the biological sample.

46-49. (canceled)

50. A method comprising, performing by a computer system:
loading, into a memory of the computer system, N machine learning language models, wherein N is an integer equal to or greater than 1;
receiving an input protein sequence of a starting protein, the input protein sequence comprised of input amino acids;
for each machine learning language model of the N machine learning language models:
executing the machine learning language model, using the input protein sequence, to obtain a likelihood of each of a set of amino acids being at each of a plurality of positions in the input protein sequence;
for each position of the plurality of positions and for each mutation of a plurality of mutations from the set of amino acids, comparing the likelihood of the mutation at the position to the likelihood of an input amino acid at the position; and
based on the comparison, identifying a set of candidate mutations that have a likelihood that is equal to or greater than the input amino acid in at least a threshold number of the N machine learning language models.

51-66. (canceled)

67. A computer product comprising a non-transitory computer readable medium storing a plurality of instructions that, when executed, cause a computer system to perform the method of claim 50.

68. A system comprising:
the computer product of claim 67; and
one or more processors for executing instructions stored on the computer readable medium.


SEQUENCE LISTING (74 sequences)

SEQ ID NO: 1 - protein, 128 aa, synthetic construct
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 2 - protein, 103 aa, synthetic construct
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 3 - protein, 128 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGGHITIFGVNIDAFDIWGQGTMVTVSS

SEQ ID NO: 4 - protein, 103 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSRTFGQGTKVEIK

SEQ ID NO: 5 - protein, 119 aa, synthetic construct [sequence omitted]

SEQ ID NO: 6 - protein, 107 aa, synthetic construct [sequence omitted]

SEQ ID NO: 7 - protein, 119 aa, synthetic construct
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCVRSDRGVAGLFDSWGQGTLVTVSS

SEQ ID NO: 8 - protein, 107 aa, synthetic construct
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 9 - protein, 127 aa, synthetic construct [sequence omitted]

SEQ ID NO: 10 - protein, 107 aa, synthetic construct [sequence omitted]

SEQ ID NO: 11 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 12 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 13 - protein, 129 aa, synthetic construct [sequence omitted]

SEQ ID NO: 14 - protein, 110 aa, synthetic construct
QSALTQPASVSGSPGQSITISCTGTSNDVGSYTLVSWYQQYPGKAPKLLIFEGTKRSSGISNRFSGSKSGNTASLTISGLQGEDEADYYCCSYAGASTFVFGGGTKLTVL

SEQ ID NO: 15 - protein, 7 aa, synthetic construct
SYNAVWN

SEQ ID NO: 16 - protein, 18 aa, synthetic construct
RTYYRSGWYNDYAESVKS

SEQ ID NO: 17 - protein, 16 aa, synthetic construct
SGHITVFGVNVDAFDM

SEQ ID NO: 18 - protein, 7 aa, synthetic construct
SNSAAWN

SEQ ID NO: 19 - protein, 18 aa, synthetic construct
RTYYRSKWYNDYAVSVKS

SEQ ID NO: 20 - protein, 16 aa, synthetic construct
GGHITIFGVNIDAFDI

SEQ ID NO: 21 - protein, 5 aa, synthetic construct
MYDMH

SEQ ID NO: 22 - protein, 16 aa, synthetic construct
AVGPSGDTYYADSVKG

SEQ ID NO: 23 - protein, 11 aa, synthetic construct
SDRGVAGLFDS

SEQ ID NO: 24 - protein, 5 aa, synthetic construct
SYDMH

SEQ ID NO: 25 - protein, 16 aa, synthetic construct
AIGTAGDTYYPGSVKG

SEQ ID NO: 26 - protein, 5 aa, synthetic construct
SYGIS

SEQ ID NO: 27 - protein, 17 aa, synthetic construct
WISTYNGNTNYAQKFQG

SEQ ID NO: 28 - protein, 18 aa, synthetic construct
DYTRGAWFGESLIGGFDN

SEQ ID NO: 29 - protein, 5 aa, synthetic construct
NYAMY

SEQ ID NO: 30 - protein, 17 aa, synthetic construct
VISYDGSNKYYADSVKG

SEQ ID NO: 31 - protein, 11 aa, synthetic construct
GSDYGDYLLVY

SEQ ID NO: 32 - protein, 5 aa, synthetic construct
TKYMT

SEQ ID NO: 33 - protein, 16 aa, synthetic construct
VLYSGGSDYYADSVKG

SEQ ID NO: 34 - protein, 21 aa, synthetic construct
DSSEVRDHPGHPGRSVGAFDI

SEQ ID NO: 35 - protein, 11 aa, synthetic construct
RTSQSLSSYTH

SEQ ID NO: 36 - protein, 7 aa, synthetic construct
AASSRGS

SEQ ID NO: 37 - protein, 5 aa, synthetic construct
QQSRT

SEQ ID NO: 38 - protein, 11 aa, synthetic construct
RASQSISSYLN

SEQ ID NO: 39 - protein, 7 aa, synthetic construct
AASSLQS

SEQ ID NO: 40 - protein, 11 aa, synthetic construct
RASQAFDNYVA

SEQ ID NO: 41 - protein, 7 aa, synthetic construct
AASALHA

SEQ ID NO: 42 - protein, 9 aa, synthetic construct
QNYNSAPLT

SEQ ID NO: 43 - protein, 11 aa, synthetic construct
RASQGISNYLA

SEQ ID NO: 44 - protein, 7 aa, synthetic construct
AASTLQS

SEQ ID NO: 45 - protein, 9 aa, synthetic construct
QKYNSAPLT

SEQ ID NO: 46 - protein, 12 aa, synthetic construct
RASQTVSSTSLA

SEQ ID NO: 47 - protein, 7 aa, synthetic construct
GASSRAT

SEQ ID NO: 48 - protein, 8 aa, synthetic construct
QQHDTSLT

SEQ ID NO: 49 - protein, 14 aa, synthetic construct
TGTSSDVGGYNYVS

SEQ ID NO: 50 - protein, 7 aa, synthetic construct
DVSKRPS

SEQ ID NO: 51 - protein, 10 aa, synthetic construct
NSLTSISTWV

SEQ ID NO: 52 - protein, 14 aa, synthetic construct
TGTSNDVGSYTLVS

SEQ ID NO: 53 - protein, 7 aa, synthetic construct
EGTKRSS

SEQ ID NO: 54 - protein, 10 aa, synthetic construct
CSYAGASTFV

SEQ ID NO: 55 - protein, 8 aa, synthetic construct
DYKDDDDK

SEQ ID NO: 56 - protein, 6 aa, synthetic construct
HHHHHH

SEQ ID NO: 57 - protein, 9 aa, synthetic construct
YPYDVPDYA

SEQ ID NO: 58 - protein, 119 aa, synthetic construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSS

SEQ ID NO: 59 - protein, 109 aa, synthetic construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVL

SEQ ID NO: 60 - protein, 120 aa, synthetic construct
QVQLAQSGSELRKPGASVKVSCDTSGHSFTSNAIHWVRQAPGQGLEWMGWINTDTGTPTYAQGFTGRFVFSLDTSARTAYLQISSLKADDTAVFYCARERDYSDYFFDYWGQGTLVTVSS

SEQ ID NO: 61 - protein, 109 aa, synthetic construct
EVVMTQSPASLSVSPGERATLSCRARASLGISTDLAWYQQRPGQAPRLLIYGASTRATGIPARFSGSGSGTEFTLTISSLQSEDSAVYYCQQYSNWPLTFGGGTKVEIK

SEQ ID NO: 62 - protein, 7 aa, synthetic construct
ISGVGVG

SEQ ID NO: 63 - protein, 16 aa, synthetic construct
LIYWDDDKRYSPSLKS

SEQ ID NO: 64 - protein, 9 aa, synthetic construct
HSISTIFDH

SEQ ID NO: 65 - protein, 5 aa, synthetic construct
SNAIH

SEQ ID NO: 66 - protein, 17 aa, synthetic construct
WINTDTGTPTYAQGFTG

SEQ ID NO: 67 - protein, 10 aa, synthetic construct
ERDYSDYFFD

SEQ ID NO: 68 - protein, 14 aa, synthetic construct
TATSSDVGDYNYVS

SEQ ID NO: 69 - protein, 7 aa, synthetic construct
EVSDRPS

SEQ ID NO: 70 - protein, 9 aa, synthetic construct
SSYTTSSAV

SEQ ID NO: 71 - protein, 13 aa, synthetic construct
RARASLGISTDLA

SEQ ID NO: 72 - protein, 7 aa, synthetic construct
GASTRAT

SEQ ID NO: 73 - protein, 9 aa, synthetic construct
QQYSNWPLT

SEQ ID NO: 74 - protein, 6 aa, synthetic construct
QVQVSS